(19) United States
(12) Patent Application Publication
(10) Pub. No.: US 2016/0184159 A1
(43) Pub. Date: Jun. 30, 2016

(54) TRAUMATIC BRAIN INJURY TREATMENT AND PREVENTION USING CYCLIC PRESSURE THERAPY

(71) Applicant: CVAC Systems, Inc., Temecula, CA (US)

(72) Inventors: Carl E. LINTON, Temecula, CA (US); Allen RUSZKOWSKI, San Diego, CA (US)

(73) Assignee: CVAC SYSTEMS, INC., Temecula, CA (US)

(21) Appl. No.: 14/885,593

(22) Filed: Oct. 16, 2015

Related U.S. Application Data
(60) Provisional application No. 62/065,306, filed on Oct. 17, 2014.

(51) Int. Cl. A61G 10/02; G01N 33/74; G01N 33/80; A61K 38/19; A61K 38/18

(52) U.S. Cl. CPC A61G 10/023; A61K 38/193; A61K 38/1816; G01N 33/80; G01N 33/746; G01N 2800/52

(57) ABSTRACT

Methods for administering pressure changes to a patient for the treatment and prevention of a traumatic brain injury (TBI). Methods of administering Cyclic Variations in Altitude Conditioning (CVAC) Sessions in a patient prior to or subsequent to a traumatic brain injury (TBI) event for the treatment of: blood production (erythropoiesis); stem cell therapy; inflammation or swelling; ischemia; or Alzheimer's disease.

### CROSS-REFERENCE TO RELATED APPLICATIONS

This application claims priority to and the benefit of U.S. provisional application Ser. No. 62/065,306, filed on Oct. 17, 2014, entitled “Traumatic Brain Injury Treatment and Prevention Using Cyclic Pressure Therapy,” which is incorporated herein by reference in its entirety.

This application is related to U.S. patent application Ser. No. 13/213,982 (now U.S. Pat. No. 8,899,228), filed Aug. 19, 2011, which is a continuation-in-part of U.S. patent application Ser. No. 13/011,058, filed Jan. 21, 2011, which is a continuation of U.S. patent application Ser. No. 11/672,934, filed Feb. 8, 2007, which claims the benefit of U.S. Provisional Application No. 60/771,848, filed Feb. 8, 2006, U.S. Provisional Application No. 60/772,647, filed Feb. 10, 2006, U.S. Provisional Application No. 60/773,460, filed Feb. 15, 2006, U.S. Provisional Application No. 60/773,585, filed Feb. 15, 2006, U.S. Provisional Application No. 60/774,441, filed Feb. 17, 2006, U.S. Provisional Application No. 60/775,917, filed Feb. 22, 2006, U.S. Provisional Application No. 60/775,521, filed Feb. 21, 2006, U.S. Provisional Application No. 60/743,470, filed Mar. 13, 2006, U.S. Provisional Application No. 60/745,721, filed Apr. 26, 2006, U.S. Provisional Application No. 60/745,723, filed Apr. 26, 2006, U.S. Provisional Application No. 60/824,890, filed Sep. 7, 2006, U.S. Provisional Application No. 60/822,375, filed Aug. 14, 2006, U.S. Provisional Application No. 60/826,061, filed Sep. 18, 2006, and U.S. Provisional Application No. 60/826,068, filed Sep. 18, 2006, which applications are incorporated herein by reference.

This application is also related to U.S. patent application Ser. No. 13/202,543, filed on Aug. 19, 2011, which is a national stage entry under 35 U.S.C. §371 of International patent application Ser. No. PCT/US2008/054923, filed on Feb. 25, 2008, which claims the benefit of U.S. Provisional Application No. 60/891,969, filed on Feb. 26, 2007, U.S. Provisional Application No. 60/953,972, filed on Aug. 3, 2007, U.S. Provisional Application No. 60/953,973, filed on Aug. 3, 2007, and U.S. Provisional Application No. 61/025,272, filed on Jan. 31, 2008, which applications are incorporated herein by reference.

This application is also related to U.S. patent application Ser. No. 14/027,474, filed on Sep. 16, 2013, which is a continuation of U.S. patent application Ser. No. 10/659,997 (now U.S. Pat. No. 8,535,064), filed on Sep. 11, 2003, which are incorporated herein by reference.

### FIELD

The invention relates to the use of air pressure therapy for the treatment and prevention of diseases and conditions that benefit from hypoxic conditioning, in particular, treatment and prevention of traumatic brain injury (TBI) using Cyclic Variations in Altitude Conditioning (CVAC) Sessions.

### BACKGROUND

The Centers for Disease Control and Prevention estimates that the yearly incidence of sports-related and recreation-related traumatic brain injury (TBI) in the United States is between 1.6 and 3.8 million, many of which remain undiagnosed or do not result in doctor or hospital visits. In a recent 10-year period, there has been a 100% increase among 8- to 13-year-olds and a 200% increase in sports-related emergency room visits for concussion among 14- to 19-year olds. Previous research has indicated that concussions represent from 5.5% to 22% of all high school athletic injuries. Athletes who sustain a concussion may demonstrate impaired executive function, poorer processing function, decreased attention span, somatic symptoms, and, in younger populations, educational difficulties and behavioral changes. These athletes are also at risk of a second concussion.

One reason the human brain is vulnerable to damage is the propensity to oscillate in the skull following a rapid acceleration or deceleration when there is impact on the skull. The oscillation of the brain in the skull is permitted because the cerebrospinal fluid (CSF) is free-flowing and the brain effectively “floats” somewhat unrestrained in the CSF.

During rapid acceleration/deceleration of the skull, such as that which exists during an impact, there is likely a concomitant deformation of brain matter, which is one proposed mechanism of concussion and brain injury. Rapid deformation of the brain can lead to increased shear when tissues of differing mass decelerate at different rates. In a concussion, rapid deformation of brain tissue is thought to cause “diffuse mechanically induced depolarization of cortical neurons.” One hypothesized mechanism of this relationship between volume of intracranial fluid and concussion risk is that a decrease in intracranial fluid results in a relative increase in the amount of space through which the brain travels in the cranium upon impact, resulting in enhanced acceleration/deceleration of both linear and rotational injury inputs to the brain. [See Smith et al, Altitude Modulates Concussion Incidence: Implications for Optimizing Brain Compliance to Prevent Brain Injury in Athletes, Orthopaedic Journal of Sports Medicine November 2013 vol. 1 no. 6].

It has been suggested that prevention of TBIs may be related to the compliance of the intracranial fluid space. Recent research indicates that altering the cerebral outflow impedance and thus optimizing the compliance (e.g., cradling the brain to prevent excess movement) of the intracranial fluid space may be associated with the likelihood and/or severity of a concussion. If one reduces the compliance of the cranial space upon impact, differing tissue densities will likely accelerate or decelerate at the same rate, similar to having an airbag deploy or “bubble wrap” inflate and thus prevent damage to structures within a container (the brain in this example). [See Smith et al, Altitude Modulates Concussion Incidence: Implications for Optimizing Brain Compliance to Prevent Brain Injury in Athletes, Orthopaedic Journal of Sports Medicine November 2013 vol. 1 no. 6].

Traumatic brain injury (TBI) may also damage the mitochondria and is a major health and socioeconomic problem throughout the world. It is a complicated pathological process that includes primary insults and a secondary insult characterized by a set of biochemical cascades. The imbalance between a higher energy demand for repair of cell damage and decreased energy production led by mitochondrial dysfunction aggravates cell damage. [see Gutsaeva et al., Transient Hypoxia Stimulates Mitochondrial Biogenesis in Brain Subcortex by a Neuronal Nitric Oxide Synthase-Dependent Mechanism, The Journal of Neuroscience, 27 Feb. 2008, 28(9): 2015-2024]. If too many mitochondrion become dysfunctional the lack of ATP/energy may be the cause of brain damage. Those with more mitochondrial may result in a higher threshold before damage occurs. [see Sharp et al., Hypoxic Preconditioning Protects against Ischemic Brain Injury, NeuroRx. 2004 January; 1(1): 26-35].

In addition, tissues deprived of blood and oxygen after injury may suffer ischemic necrosis or infarction, often resulting in permanent tissue damage. Cerebral ischemia results from decreased blood and oxygen flow which is often followed by some degree of brain damage. The decrease in blood flow and oxygenation may be the result of occlusion or rupture of vessels from physical traumas, such as TBI. Cerebral ischemia is often termed a “stroke”.

TBI may also include inflammation and swelling within the head. Inflammation may include increased swelling, temperature, pain, and some loss of function in the affected area. Pharmaceuticals such as steroids and steroid-based anti-inflammatories may also be used to treat inflammatory conditions or swelling or combinations thereof, but pharmaceuticals can bring on additional concerns due to negative side-effects from the compound itself, length of treatment, and unforeseen, individual reactions to the drugs. For example, glucocorticoids, administered to relieve inflammation and swelling, have known detrimental effects associated with extended use such as inhibition of bone formation, suppression of calcium absorption, delayed wound healing, and other immunosuppressive effects. Thus their effectiveness and their potential for long-term therapy is limited. Furthermore, compliance with pharmaceutical regimens can be difficult over long term or even short term therapy. While many different drugs can be used to treat the causes and symptoms of inflammation and swelling, there is a need for therapies which can be useful for further ameliorating the damage and resultant effects of inflammation and swelling beyond existing pharmaceutical and physical therapy remedies.

In the early 1990's, researchers and the public began to focus on stem cells and their potential use for treatment of diseases. Generally, stem cell therapies are limited by the supply of autologous stem cells. Initial efforts primarily utilized bone marrow aspiration techniques to harvest autologous stem cells (stem cells from one's own body) and heterologous stem cells (stem cells from a source other than one's own body). More recently, stem cells are preferably collected from a patient through a process called mobilization. Mobilization is achieved with the use of cytotoxic drugs and/or growth factors which are administered in very high dosages. Stem cell engraftment has a low rate of success, and many of the stem cells from the mobilization do not successfully implant despite the volume of cells administered, thus lengthening the recovery period as well as significantly increasing the costs associated with the procedure. [Josh et al., Immunological properties of mononuclear cells from blood stem cell harvests following mobilization with erythropoietin+G-CSF in cancer patients, Cytotherapy 2(1):15-24 (2002)].

TBI may also be related to memory loss brought on by Alzheimer's disease. There have been studies on a relationship between brain injury and Alzheimer's disease through the apolipoprotein E (APOE) gene, in particular, the APOE e4 allele. [see Horsburgh et al., The role of apolipoprotein E in Alzheimer's disease, acute brain injury and cerebrovascular disease: evidence of common mechanisms and utility of animal models, Neurobiol Aging 2000; 21:245-55]. Cerebral amyloid angiopathy (CAA) is a pathological condition characterized by the deposition of amyloid in cerebral cortical and leptomeningeal blood vessels. Among head injured patients, CAA was considerably more likely to occur in those with a genetic predisposition conferred by possession of the APOE e4 allele. Of particular interest is the possibility that the presence of CAA in patients who experience a head injury may influence the clinical outcome. For example, blood vessels laden with amyloid may be more prone to rupture after trauma, resulting in hemorrhage. [Leclercq et al., Cerebral amyloid angiopathy in traumatic brain injury: association with apolipoprotein E genotype, J Neurol Neurosurg Psychiatry 2005; 76:229-233].

Treatment Options and Needs

Current methods for prevention and treatment of brain injuries are few. Similarly, known methods of improving erythropoiesis are few and require the administration of pharmaceuticals or other factors such as EPO to stimulate red blood cell production and increase blood volume. Known methods of stem cell mobilization, engraftment and recovery also primarily require the administration of pharmaceuticals and methods for improving efficiency of, and recovery from, stem cell transplantation are few. As stated, pharmaceutical intervention is typically the remedy of choice for all of the aforementioned indications. Additionally, regular and increased administration of exogenous hormones and molecules such as EPO can have detrimental results such as a decrease in oxygen carrying capacity of the blood due to extreme increases in hematocrit and increases in blood viscosity. Additionally, stem cell mobilization methods also require the use of large doses of toxic pharmaceuticals and growth factors.

Thus there is a need for therapies which improve the treatment of the aforementioned indications. Further there is a need for additional therapies to work simultaneously or in concert with traditional methods for treating TBI, improving erythropoiesis, and facilitating stem cell therapy. There is also a need for therapies without the potential negative side-effects of pharmaceutical and growth factor regimens. Alternatively, there is a need for such therapies that could lessen the negative side-effects of pharmaceutical and growth factor regimens by altering such regimens, that could work beneficially with pharmaceutical and growth factor regimens, or that could work synergistically when used in combination with pharmaceutical and growth factor regimens.

### SUMMARY

The invention generally relates to the use of air pressure therapy for the treatment and prevention of diseases, conditions, and disorders, and more specifically to the treatment of traumatic brain injury (TBI) using hypoxic conditioning and/or total body vaso-pneumatic compression. Methods according to embodiments provide for administering pressure changes to a user for the treatment of TBI. Treatment as used herein includes application of the disclosed methodologies for prevention, prophylactic treatment, current treatment, amelioration, alleviation and/or recovery of the disease, condition, or disorder.

The present invention provides for a method of administering pressure changes to a patient for the purpose of increasing blood production and erythropoiesis that may prevent or reduce traumatic brain injury (TBI) through the use of cyclic pressure therapy to increase blood production to create a “tighter fit” between the brain and skull. This may be done with the administration of Cyclic Variations in Altitude Conditioning (CVAC) Sessions for the improvement of red blood cell production. In an embodiment of the invention, at least one CVAC session is administered to improve blood production. CVAC sessions may be administered in defined intervals or at random occurrences. The effect of such administration is to increase blood production.

The present invention provides for a method of administering pressure changes to a user for the treatment, prevention, and amelioration of inflammation and swelling. Application of the disclosed methodologies can aid in recovery from inflammation and swelling, the increased drainage of fluids or toxins or combinations thereof from the affected areas, and the modulation of genetic elements and resultant expression of molecules involved in inflammatory and immune responses.

The present invention provides for a method of administering pressure changes (CVAC) to a user for the prevention, treatment, and amelioration of ischemic disease and complications associated with or arising from such disease. Ischemic disease encompasses cerebral ischemia, strokes, and myriad associated cerebral conditions associated with blockages of blood vessels, ruptures of vessels, loss in blood pressure, and damage to surrounding tissues. Application of the disclosed methodologies helps to prevent the onset of ischemic disease, treats asymptomatic and symptomatic disease, and aids in recovery from ischemic disease, ischemic events, complications associated with or arising from ischemia.

The present invention can prevent and/or treat traumatic brain injury (TBI) through the use of cyclic pressure therapy to produce more mitochondria. The primary increase in mitochondria is in the hippocampus. The hippocampus is known to be a source of neuronal stem cells. So if the brain has a ready supply of stem cells available to replace damaged dying cells, those cells may be able to replace the dying damaged cells before cell death in time to transfer the functional programming, perhaps which includes memory to the new stem cells. The programming memory may reside in the RNA and the RNA gets transferred from the inflamed dying cell to the new stem cell. Then the stem cell takes over the function of the old dead cell. The lymphatic stimulation resulting from the cyclic pressure therapy mobilizes the stem cell to the site of the inflamed dying cell and washes away the old dead cell after it has transferred its programming to the new cell.

The present invention provides for a method of administering pressure changes to a user for the prevention, treatment, and/or amelioration of memory loss due to Alzheimer's disease. Further, application of disclosed methodologies can aid in the prevention of amyloid plaque formation, the increased exchange of fluids, toxins or combinations thereof from the affected areas, and/or the modulation of genetic elements and resultant expression of molecules involved in inflammatory and immune responses that may be involved in Alzheimer's disease.

Pressure vessels have been used on humans for various purposes for many years. However, the typical approach to the use of these pressure vessels was to subject a person to sustained periods in a pressurized environment. Contrary to this typical approach, the underlying theory for the present invention is that the benefits to be derived from exposure to a pressure vessel results not from sustained exposure to a pressurized environment, but rather, to the transition between various simulated altitudes. In other words, one of the goals of the present invention is to provide conditioning and treatment to a human body through cyclic variations in altitude. By subjecting a person to transitions in simulated altitudes, the person is subjected to more than the mere exposure to a pressurized environment. In cyclic variations in altitude conditioning and treatment, the person is exposed to transitions in pressure, temperature and oxygen levels. It is through this use of varying cyclic patterns of transitions between simulated altitudes that a person can more effectively derive the benefits of conditioning and treatment from a pressure vessel.

In some embodiments, it is believed that the transition between simulated altitudes creates a polarity shift in the cell walls of the human body, and that these transitions also impact the bioelectric frequency of the human body. Thus, the past practice of subjecting a person to sustained periods in a pressurized environment did not subject the person to multiple transitions between altitudes.

A CVAC session can include a set of targets which are pressures found in the natural atmosphere. A CVAC session can include start and end points and more than one target executed between the start and end points. These targets are delivered in a precise order, and are executed in a variety of patterns including, but not limited to, cyclic, repeating or linear variations or any combination thereof. The starting points and ending points in any CVAC session are preferably the ambient pressure at the delivery site. The targets in each CVAC session are connected or joined together by defined transitions. These transitions are either rises in pressure or falls in pressure, or a combination of the two. In some embodiments, additional targets which modulate time, temperature, or humidity are also executed concurrently, sequentially, or at other intervals with the pressure targets.

An embodiment of the invention includes the administration of CVAC sessions for the treatment of TBI. Further embodiments of the invention can include administering the CVAC sessions prior to TBI events and subsequent to TBI events to treat, prevent, or ameliorate the effects of TBI. Even further embodiments may include administration of CVAC sessions prior to and after surgeries related to TBI for the prevention and amelioration of detrimental effects resulting from such surgeries.

In some embodiments, one or more targets of a CVAC session can include pressure, temperature, time, and/or humidity parameters. Parameters of targets and sessions can be customized to individual needs. In still further embodiments, including the aforementioned aspects and embodiments, CVAC sessions are administered in combination with pharmaceutical regimens for the treatment of TBI or TBI prevention. Further embodiments, including the aforementioned embodiments and aspects, include administration of CVAC sessions in combination with alternative therapies and non-pharmaceutical therapies for the treatment of the aforementioned TBI conditions, and syndromes as defined herein.

### BRIEF DESCRIPTION OF THE DRAWINGS

FIG. 1A depicts a graphed profile of various pressures applied over time during an exemplary CVAC session. The Y-axis represents atmospheric pressure levels and the X-axis represents time. The varying pressures, as indicated by the changes in values on the Y-axis, were applied for various lengths of time, as indicated by changes in values on the X-axis. The exemplary CVAC session depicted in FIG. 1A was 20 minutes in length.

FIG. 1B depicts a different graphed profile of the pressures applied over time during another exemplary CVAC session. The Y-axis represents atmospheric pressure levels and the X-axis represents time. Different pressures were applied, as indicated by changes in value on the Y-axis, and for various lengths of time, as indicated by the changes in values on the X-axis. This exemplary CVAC session was 20 minutes in length.

FIG. 2 depicts a chart summarizing the serum lipid levels from 7 subjects following treatment with CVAC sessions. Total cholesterol, triglycerides, HDL, VLDL, and LDL levels are represented prior to and following administration of CVAC sessions for 40 minutes, twice a week throughout the study period.

FIG. 3 depicts a chart summarizing testosterone levels from 7 subjects following treatment with CVAC sessions. Total testosterone, free testosterone, and ratios of free testosterone to total testosterone are represented prior to and following administration of CVAC sessions for 40 minutes, twice a week throughout the study period.

FIG. 4 is a graphical illustration of various pressures applied over time during a CVAC session using profile BRG at tier 2.

FIG. 5 is a graphical illustration of various pressures applied over time during a CVAC session using profile RBG at tier 2.

FIG. 6 is a graphical illustration of various pressures applied over time during a CVAC session using profile GRB at tier 2.

FIG. 7 is a graphical illustration of various pressures applied over time during a CVAC session using profile sham at tier 2.

FIG. 8 is a graphical illustration of various pressures applied over time during a CVAC session using profile BRG at tier 3.

FIG. 9 is a graphical illustration of various pressures applied over time during a CVAC session using profile RBG at tier 3.

FIG. 10 is a graphical illustration of various pressures applied over time during a CVAC session using profile GRB at tier 3.

FIG. 11 is a graphical illustration of various pressures applied over time during a CVAC session using profile sham at tier 3.

FIG. 12 is a graphical illustration of various pressures applied over time during a CVAC session using profile BRG at tier 4.

FIG. 13 is a graphical illustration of various pressures applied over time during a CVAC session using profile RBG at tier 4.

FIG. 14 is a graphical illustration of various pressures applied over time during a CVAC session using profile GRB at tier 4.

FIG. 15 is a graphical illustration of various pressures applied over time during a CVAC session using profile sham at tier 4.

FIG. 16 is a graphical illustration of various pressures applied over time during a CVAC session using profile BRG at tier 5.

FIG. 17 is a graphical illustration of various pressures applied over time during a CVAC session using profile RBG at tier 5.

FIG. 18 is a graphical illustration of various pressures applied over time during a CVAC session using profile GRB at tier 5.

FIG. 19 is a graphical illustration of various pressures applied over time during a CVAC session using profile sham at tier 5.

FIGS. 20-23 are graphical illustrations of various pressures applied over time during a CVAC session using profile GLESS at tiers 2 through 5, respectively.

FIGS. 24-27 are graphical illustrations of various pressures applied over time during a CVAC session using profile BMORE at tiers 2 through 5, respectively.

FIGS. 28-31 are graphical illustrations of various pressures applied over time during a CVAC session using profile RMORE at tiers 2 through 5, respectively.

FIGS. 32-36 are graphical illustrations of various pressures applied over time during a CVAC session using profiles T1#1EXT, T1#2EXT, T1#3EXT, T1#4EXT, and T1#5EXT, respectively.

FIGS. 37-41 are graphical illustrations of various pressures applied over time during a CVAC session using profiles T2Hammer, T3Hammer, T4Hammer, T5Hammer and T6Hammer, respectively.

FIG. 42 is a graphical illustration of various pressures applied over time during a CVAC session using profile Turbo6 BGR.

FIG. 43 is a graphical illustration of various pressures applied over time during a CVAC session using profile Turbo6 BLESS.

### DETAILED DESCRIPTION

Cyclic Variations in Altitude Conditioning (CVAC)

Studies have shown that CVAC treats many of the condition associated with Traumatic brain injury (TBI). The benefits may include an increase in mitochondrial function and perhaps mitochondrial biogenesis [Marquez et al., Cyclic hypobaric hypoxia improves markers of glucose metabolism in middle-aged men, High Alt Med Biol 14:263-272, 2013], including a piezoelectric effect of the liquid crystal structure of cell membranes [Crane et al., Massage Therapy Attenuates Inflammatory Signaling After Exercise-Induced Muscle Damage, Sci. Transl. Med. 4, 119ra13 (2012)]; an increase in blood production; an increase in EPO production; stem cell therapy, ischemia treatment and other benefits.

CVAC sessions are believed to act like a vaso-pneumatic pump on the user's body, thus stimulating flow of fluids in the body, including but not limited to, blood and other fluids. The negative and positive pressures imposed by the CVAC session affect the fluid flow or movement within a body, thus improving the delivery of beneficial nutrients, immune factors, blood, and oxygen while also improving the removal of harmful toxins, fluids, and damaged cells or tissues.

CVAC sessions can be administered for the treatment of hypertension, blood production, stem cell therapy, spinal cord injury, intervertebral disc therapy, inflammation, wound healing, ischemic disease, diabetes and associated complications, Alzheimer's disease, and cancer. One or more CVAC sessions can also be administered for the treatment, prevention and/or amelioration of symptoms and/or secondary injuries associated with traumatic brain injury (TBI).

Traumatic Brain Injury (TBI)

Traumatic brain injury (TBI), also known as intracranial injury, occurs when an external force traumatically injures the brain. TBI can occur as a consequence of a focal impact upon the head, by a sudden acceleration/deceleration within the cranium, or by a complex combination of both movement and sudden impact. In addition to the damage caused at the moment of injury, brain trauma may include a secondary injury that take place in the minutes and days following the injury. These processes, which include alterations in cerebral blood flow and the pressure within the skull, contribute substantially to the damage from the initial injury.

The most common causes of TBI in the U.S. include sports, transportation accidents, construction, and violence. [Kushner D (1998), Mild traumatic brain injury: Toward understanding manifestations and treatment, Archives of Internal Medicine 158 (15): 1617-24; Faul et al. (2010), Traumatic Brain Injury in the United States: Emergency Department Visits, Hospitalizations, and Deaths, 2002-2006. National Center for Injury Prevention and Control, Centers for Disease Control and Prevention.]. The estimates that between 1.6 and 3.8 million traumatic brain injuries each year are a result of sports and recreation activities in the US. [Traumatic brain injury, Centers for Disease Control and Prevention, National Center for Injury Prevention and Control. 2007]. Firearms and blast injuries from explosions are other causes of TBI, which is a leading cause of death and disability in war zones. [Park et al., (April 2008), Traumatic brain injury: Can the consequences be stopped?, Canadian Medical Association Journal 178 (9): 1163-70]. According to Representative Bill Pascrell (Democrat, N.J.), traumatic brain injury is “the signature injury of the wars in Iraq and Afghanistan.” [Pentagon Told Congress It's Studying Brain-Damage Therapy, ProPublica. Retrieved 2011-01-23]

A person with a moderate or severe TBI may have a headache that does not go away, repeated vomiting or nausea, convulsions, an inability to awaken, dilation of one or both pupils, slurred speech, aphasia (word-finding difficulties), dysarthria (muscle weakness that causes disordered speech), weakness or numbness in the limbs, loss of coordination, confusion, restlessness, or agitation. [NINDS Traumatic Brain Injury Information Page, National Institute of Neurological Disorders and Stroke. Sep. 15, 2008.]

The type, direction, intensity, and duration of forces all contribute to the characteristics and severity of TBI. [Maas et al., (August 2008), Moderate and severe traumatic brain injury in adults, Lancet Neurology 7 (8): 728-41]. Forces that may contribute to TBI include angular, rotational, shear, and translational forces. [Hardman et al., (2002), Pathology of head trauma, Neuroimaging Clinics of North America 12 (2): 175-87, vii].

Even in the absence of an impact, significant acceleration or deceleration of the head can cause TBI; however in most cases a combination of impact and acceleration is probably to blame. [Hardman et al., (2002), Pathology of head trauma, Neuroimaging Clinics of North America 12 (2): 175-87, vii]. Forces involving the head striking or being struck by something, termed contact or impact loading, are the cause of most focal injuries, and movement of the brain within the skull, termed noncontact or inertial loading, usually causes diffuse injuries. [Saatman et al., (2008), Classification of traumatic brain injury for targeted therapies, Journal of Neurotrauma 25 (7): 719-38].

A large percentage of the people killed by brain trauma do not die right away but rather days to weeks after the event. [Sauaia et al. (February 1995), Epidemiology of trauma deaths: A reassessment, The Journal of Trauma 38 (2): 185-93]. Primary brain injury (the damage that occurs at the moment of trauma when tissues and blood vessels are stretched, compressed, and torn) is not adequate to explain this deterioration; rather, it is caused by secondary injury, a complex set of cellular processes and biochemical cascades that occur in the minutes to days following the trauma. [Xiong et al., (2000), Mitochondrial dysfunction following traumatic brain injury, In Miller L P and Hayes R L, eds. Co-edited by Newcomb J K. Head Trauma: Basic, Preclinical, and Clinical Directions. New York: John Wiley and Sons, Inc. pp. 257-80]. These secondary processes can dramatically worsen the damage caused by primary injury [Park et al., (April 2008), Traumatic brain injury: Can the consequences be stopped?, Canadian Medical Association Journal 178 (9): 1163-70] and account for the greatest number of TBI deaths occurring in hospitals. [Ghajar J (September 2000), Traumatic brain injury, Lancet 356 (9233): 923-29].

Secondary injury events include damage to the blood-brain barrier, release of factors that cause inflammation, free radical overload, excessive release of the neurotransmitter glutamate (excitotoxicity), influx of calcium and sodium ions into neurons, and dysfunction of mitochondria. Injured axons in the brain's white matter may separate from their cell bodies as a result of secondary injury, potentially killing those neurons. [Park et al., (April 2008), Traumatic brain injury: Can the consequences be stopped?, Canadian Medical Association Journal 178 (9): 1163-70]. Other factors in secondary injury are changes in the blood flow to the brain; ischemia (insufficient blood flow); cerebral hypoxia (insufficient oxygen in the brain); cerebral edema (swelling of the brain); and raised intracranial pressure (the pressure within the skull). [Scalea T M (2005), Does it matter how head injured patients are resuscitated?, In Valadka A B, Andrews B T. Neurotrauma: Evidence-based Answers to Common Questions. Thieme. pp. 3-4]. Intracranial pressure may rise due to swelling or a mass effect from a lesion, such as a hemorrhage. [Salomone et al., (2004), Prehospital care, In Moore E J, Feliciano D V, Mattox K L. Trauma. New York: McGraw-Hill, Medical Pub. Division. pp. 117-8]. As a result, cerebral perfusion pressure (the pressure of blood flow in the brain) is reduced; ischemia results. [Ghajar J (September 2000), Traumatic brain injury, Lancet 356 (9233): 923-29.]. When the pressure within the skull rises too high, it can cause brain death or herniation, in which parts of the brain are squeezed by structures in the skull. [Morley et al., (September 2008), Mannitol for traumatic brain injury: Searching for the evidence, Annals of Emergency Medicine 52 (3): 298-300].

Hypoxic conditioning protocols utilize static pressures for blocks of time ranging from 30 minutes to an hour or more to achieve the desired and reported responses. Hypoxic conditioning may be provided by decreased oxygen levels in the atmosphere or by a reduction in atmospheric pressure (hypobaric conditions), thus reducing the availability of oxygen for efficient respiration. Both methods can provide beneficial results including treatment of tissue due to TBI and protection of tissues from TBI.

Moderate static hypoxic preconditioning is known to provide protection from ischemic damage via tolerance. When the environmental oxygen levels are reduced (hypoxia), downstream effects include protection from damage due to subsequent hypoxia. This tolerance is not yet completely understood, but it has been linked to various cellular mechanisms and molecules, including, but not limited to, molecules such as erythropoietin (EPO), hypoxia-inducible factor (HIF), Tumor Necrosis Factor (TNF), glycogen, lactate, and others. Additionally, beneficial static hypoxic conditioning is not purely additive. Administration of sequential sessions can have detrimental effects. Oxygen concentrations that are too low result in detrimental effects to the tissues as well as the entire body. Similarly, hypoxic conditioning of longer durations can have detrimental effects in addition to providing some desired beneficial effects [see Sharp, F., et al., Hypoxic Preconditioning Protects against Ischemic Brain Injury, NeuroRx: J. Am. Soc. Exp. Neuro., Vol. 1: 26-25 (2004)].

Initial understanding in the art about the effects of hypoxia focused on increased oxygenation of the blood via increased production of red blood cells mediated by increases in EPO production. While increases in EPO production are believed to increase red blood cell production, its effects are not limited to this activity. Additional studies also show protective activity for EPO in white and gray matter (brain and spinal cord tissue), inflammatory and demyelinating conditions, and other various ischemic events. [Eid, T. and Brines, M., Recombinant human erythropoietin for neuroprotection: what is the evidence?, Clin. Breast Cancer, 3 Suppl. 3:S109-15, December 2002]. Furthermore, molecules such as HIF, induced by hypoxia, regulate EPO production in addition to a variety of other activities including metabolism, angiogenesis, and vascular tone—the stimulation of which may all play a role in protecting tissue from subsequent hypoxic damage both prophylactically and post-ischemic or traumatic events. [Eckardt K U, Kurtz, A., Regulation of erythropoietin production, Eur. J. Clin. Invest., (Supp. 3):13-19, (2005)].

In addition to EPO administration, therapies such as oxygen deprivation at static air pressures and static blocks of time are known to provide some beneficial effects for increasing red blood cell production, oxygenation of the blood and hematocrit. [Heinicke K, et al., Long-term exposure to intermittent hypoxia results in increased hemoglobin mass, reduced plasma volume, and elevated erythropoietin plasma levels in man, Eur. J. Appl. Physiol., 88(6):535-43 (2003)]. While oxygen deprivation of the body or specific tissues can cause tissue damage, and even death, controlled deprivation of oxygen to the body or specific tissues or a combination thereof may be beneficial when imposed for specific periods of time under particular conditions. Static hypoxic conditioning may be provided by decreased oxygen levels in the atmosphere or by a reduction in atmospheric pressure (hypobaric conditions), thus reducing the availability of oxygen for efficient respiration. Both methods can provide beneficial results including prevention of damage due to inflammation and swelling.

Further there is a need for such therapies without the potential negative side-effects of strictly pharmaceutical regimens. Alternatively, there is a need for such therapies that could lessen the negative side-effects of pharmaceutical regimens by altering pharmaceutical regimens, could work beneficially with pharmaceutical regimens, or could work synergistically when used in combination with pharmaceutical regimens. There is a further need for hypobaric or hypoxic conditioning which maximizes the beneficial effects within short treatment periods that do not lead to the detrimental effects of such conditioning as found with current methods of static hypobaric conditioning. There is a further need for such hypobaric or hypoxic conditioning that utilizes multiple and/or varying pressures throughout the conditioning. There is yet a further need for hypobaric or hypoxic conditioning that incorporates vaso-pneumatic effects in addition to the hypoxic considerations.

The apparatus and methods of treatment described herein may provide for such needs and may do so in a manner unique and generally advantageous compared to all previous forms of hypobaric conditioning. Similarly, the apparatus and methods described herein can provide for vaso-pneumatic effects in a manner both unique and generally advantageous to previous vibrational therapies and endermologie. Additionally, CVAC sessions can provide for vaso-pneumatic beneficial effects. Although not limited, CVAC sessions are believed to act like a vaso-pneumatic pump on the user's body, thus stimulating flow of fluids in the body, including but not limited to blood and lymphatic fluids. The negative and positive pressures imposed by the CVAC session can affect the fluid flow or movement within a body, thus improving the delivery of beneficial nutrients, immune factors, blood, and oxygen while also improving the removal of harmful toxins, fluids, and damaged cells or tissues. Furthermore, the vaso-pneumatic effects generated during any given CVAC session can exert pressures on the body and tissues of a user. CVAC can also provide similar application of force and/or transfer of mechanical energy into the cells and tissue of a user via vaso-pneumatic pressure. However, CVAC sessions provide for a unique application of varying pressure changes and times superior to the static application of force described previously, thus providing the beneficial effects of physical forces in a novel and generally advantageous way. CVAC has benefits over traditional Normobaric hypoxia [Uhlik et al., Normobaric hypoxia induces mild damage to epithelium of terminal bronchioles in rabbits (ultrastructural study), Vet. Med. —Czech, 50, 2005 (10): 432-438], [Hemmingsson et al., Lower exhaled nitric oxide in hypobaric than in normobaric acute hypoxia, Respir. Physiol. Neurobiol. (2009)].

CVAC uses a Pressure Vessel Unit (PVU) for facilitating pressure changes accurately and quickly in the environment surrounding a user. A PVU can provide both reduced and increased atmospheric pressures. An example of a unique PVU and associated methods for controlling the pressure within such a PVU are described in U.S. Pat. No. 8,535,064 and International Application No. WO/2005/035068, the disclosures of which are incorporated herein by reference. A variety of PVUs may be used in conjunction with the methods disclosed herein, including but not limited to those described in the U.S. Pat. No. 8,535,064 and WO/2005/035068, such as variable or fixed pressure and temperature hypobaric units. Other pressure units or chambers known to those of skill in the art and can be adapted for use with the disclosed methodologies.

Methodology of the Cyclic Variations in Altitude Conditioning (CVAC) Program:

The methodology of the methods of treatment described herein encompasses a set of pressure targets with defined transitions. Additional targets can be included such as temperature or humidity, and these targets can be implemented concurrently, prior to, or subsequent to the pressure targets. In some embodiments, the permutations of targets can be customizable to the individual and condition to be treated. Some of the terms relating to this methodology are defined below for a better understanding of the methodology as used in the context of the present invention.

A CVAC Program:

The CVAC program includes a set of sessions, which are administered to the user as a serial round or cycle. This means that a user may have a session that they start and repeat a given number of times and then proceed to the next scheduled session which will be repeated a given number of times. A program may contain a set of one or more sessions, each of which can have a repetition schedule. The sessions can be delivered in a scheduled order, which repeats itself like a loop such that the user is administered one session at a time for a specified number of times. The user may then be administered the next scheduled session a specified number of times. This process can be repeated until the user is administered the last element of the scheduled sessions set. When the requisite repetitions have been accomplished, the process can repeat itself beginning at the first element of the scheduled sessions set. A session or groups of sessions may be repeated multiple times before changing to a subsequent session or group of sessions, however, sessions may also be administered as few as one time before beginning the next session in the sequence. Subsequent sessions can contain targets that are identical to the previous session, or they can implement new permutations of desired targets. The combination of sessions and targets within sessions can be customizable based on the desired physiological outcome and assessment of the user. Alternatively, a user may also modulate the parameters of a CVAC session, in certain embodiments from within the unit, thus providing for real-time user feedback and alterations. As used in reference to a parameter of a CVAC session, modulation includes any changes, positive and negative, made to the parameters of the CVAC session. The parameters are described herein. This comprises a Cyclic Variations in Altitude Conditioning (CVAC) Program.

A CVAC Session:

A CVAC Session can include of a set of targets which are multiple atmospheric pressures, and a CVAC session includes start and end points, and more than one target which is executed between the start and end points. These targets are delivered in an order that may vary and are executed in a variety of patterns including, but not limited to, cyclic, repeating, and/or linear variations. When a target is executed as contemplated herein, executed includes a change in pressure from one pressure value to another pressure value within a CVAC device as also described herein. The methodologies described herein provide superior benefits compared to previously described static hypobaric pressure therapies in multiple ways, which can include reduced time frames of application and unique variations and combinations of atmospheric pressures. Furthermore, CVAC sessions can also provide beneficial effects via the vaso-pneumatic properties associated with the application of such sessions.

In some embodiments, at least one of the starting point and the ending point in any CVAC Session is the ambient pressure at the delivery site. The targets inherent in any CVAC Session are connected or joined together by defined transitions. These transitions can include an increase in pressure (descent), a decrease in pressure (ascent), or a combination thereof. The nature of any transition may be characterized by the function of “delta P/T” (change in pressure over time). Transitions may be linear or produce a waveform. In some embodiments, all transitions produce a waveform. Suitable waveforms are sine, trapezoidal and square.

In some embodiments, additional targets which modulate time, temperature, and/or humidity run concurrently, sequentially, or at other intervals with the pressure targets when such additional targets and conditions are desired. In some embodiments, the entire collection of targets and/or transitions are delivered in a twenty minute CVAC Session, although the time of each session may vary in accordance with the desired outcome of the administration of the CVAC Session(s). For example, CVAC sessions may be administered over minute increments such as 5, 10, 15, 16, 17, 18, 19, 20, 25, 30 minutes or more. The length of each CVAC Session is customizable for each user.

CVAC sessions are administered preferably for at least 10 minutes, and more preferably at least 20 minutes, with variable frequency. Additional administration periods may include, but are not limited to, about 10 minutes, about 20 minutes, about 30 minutes, about 40 minutes, about 60 minutes, between 10 and 20 minutes, between 20 and 30 minutes, between 30 and 60 minutes, and between 60 and 120 minutes. Frequencies of sessions or series of sessions may include, but are not limited to, daily, monthly, or when medically indicated or prescribed. The frequency and duration of the sessions can be altered to suit the medical condition to be treated, and CVAC sessions may be administered as single sessions, or as a series of sessions, preferably with a Set-Up Session as described herein. For example, the frequency of sessions or series of sessions can be administered 3 times a week for 8 weeks, 4 times a week for 8 weeks, 5 times a week for 8 weeks, or 6 times a week for 8 weeks. Additional frequencies can be easily created for each individual user. Similarly, the targets in the sessions can also be altered or adjusted to suit the individual and medical condition to be treated. If at any time the user or attendant determines that the session is not being tolerated well, preferably an abort may be initiated and the user brought down safely and exited. The permutations of targets can be customized to the individual, and may again be identified with the help of any person skilled in the art, such as a treating physician. Furthermore, the variations may be administered in regular intervals and sequence, as described, or in random intervals and sequence. The variations in number, frequency, and duration of targets and sessions can be applied to all methods of treatment with CVAC described herein.

In some embodiments, the CVAC session includes a set of predetermined pressure targets with predetermined defined transitions. In some embodiments, the CVAC session can include any suitable number of cyclic altitude changes. For example, in some embodiments, the CVAC session can include 50, 100, 200, 300, 400, 500 or more cyclic altitude changes during a single CVAC session (e.g., during a CVAC session of 10, 15, 20, 25, 30, 35, 40, 45 or more minutes in length. In another example, the CVAC session can include between about 100 and about 500 cyclic altitude changes, between about 200 and about 400 cyclic altitude changes, or between about 300 and about 500 cyclic altitude changes.

In some embodiments, each CVAC session includes a plurality of targets executed within a short interval of the overall session duration. For example, in some embodiments, each CVAC session includes a plurality of targets executed during an interval of twenty (20) seconds, forty (40) seconds, one (1) minute, two (2) minutes, five (5) minutes, or any suitable interval, of the overall CVAC session duration. For example, during such an interval, the CVAC session can include about 10, 20, 30, 40, 50 or more cyclic pressure changes.

As used herein, the terms “about” and/or “approximately” when used in conjunction with numerical values and/or ranges generally refer to those numerical values and/or ranges near to a recited numerical value and/or range. For example, in some instances, “about 40 [units]” can mean within ±25% of 40 (e.g., from 30 to 50). In some instances, the terms “about” and “approximately” can mean within ±10% of the recited value. In other instances, the terms “about” and “approximately” can mean within ±5%, ±4%, ±3%, ±2%, ±1%, less than ±1%, or any other value or range of values therein or therebelow. The terms “about” and “approximately” may be used interchangeably.

A Set-Up Session:

The Set-Up Session may also be considered a Program. It is a single session designed to prepare a new user for the more aggressive maneuvers or transitions encountered in the subsequent Sessions that the user will undergo. The Set-Up session accounts for all ages and sizes and conditions, and assumes a minimal gradient per step exercise that allows the ear structures to be more pliant and to allow for more comfortable equalization of pressure in the ear structures. The purpose of the Set-Up session is to prepare a new user for their custom Program based upon the group into which they have been placed.

The function of the Set-Up session is to qualify a user as being capable of adapting to multiple pressure changes in a given Session with acceptable or no discomfort. Set-Up session transitions may be linear or produce a waveform. In some embodiments, all transitions during a Set-Up session are linear. This is accomplished by instituting a gradient scale increase in pressure targets from very slight to larger increments with slow transitions increasing until a maximum transition from the widest difference in pressure targets is accomplished with no discomfort. The structure of a Set-Up session according to an embodiment is as follows: as with any Session, the starting point and ending point can both be ambient pressure. A target equivalent to about 1000 ft above ambient pressure is accomplished via a smooth linear transit. A second target equivalent to about 500 ft less than the first target is accomplished via a slow to moderate transit. These two steps are repeated until the user returns a “continue” or “pass” reply via an on-board interface. When the user has indicated that they are prepared to continue, the initial target (1000 ft above ambient) is increased by a factor of 500 ft, making it about 1500 ft. The secondary target (500 ft less than the first target) remains the same throughout the session until the exit stage is reached. In this example, each time the user indicates that they are ready to increase their gradient, the target is increased by a factor of about 500 ft. At this time, the transits remain the same but the option of increasing gradient (shorter time factor) in the transits is available. In some embodiments, a user can optionally resume a lower gradient, if desired. There can be an appropriate icon or pad that allows for this option on the on-board interface display screen.

In some embodiments, the Set-Up Session lasts no longer than 20 minutes. A Set-Up session typically runs for twenty minutes maximum and executes a final descent to ambient atmospheric pressure upon beginning the last transit. The Set-Up session is a new user's Program until the user is able to fully complete the Set-Up session (that is to continue the targets and transits to the highest gradient) with no interrupts or aborts. When administering CVAC sessions for medical treatment, Set-Up sessions may be customized to suit the requirements of their medical condition. The determination of the appropriate Set-Up Session can be made with guidance from or consultation with a user's qualified health professional, such as a treating physician.

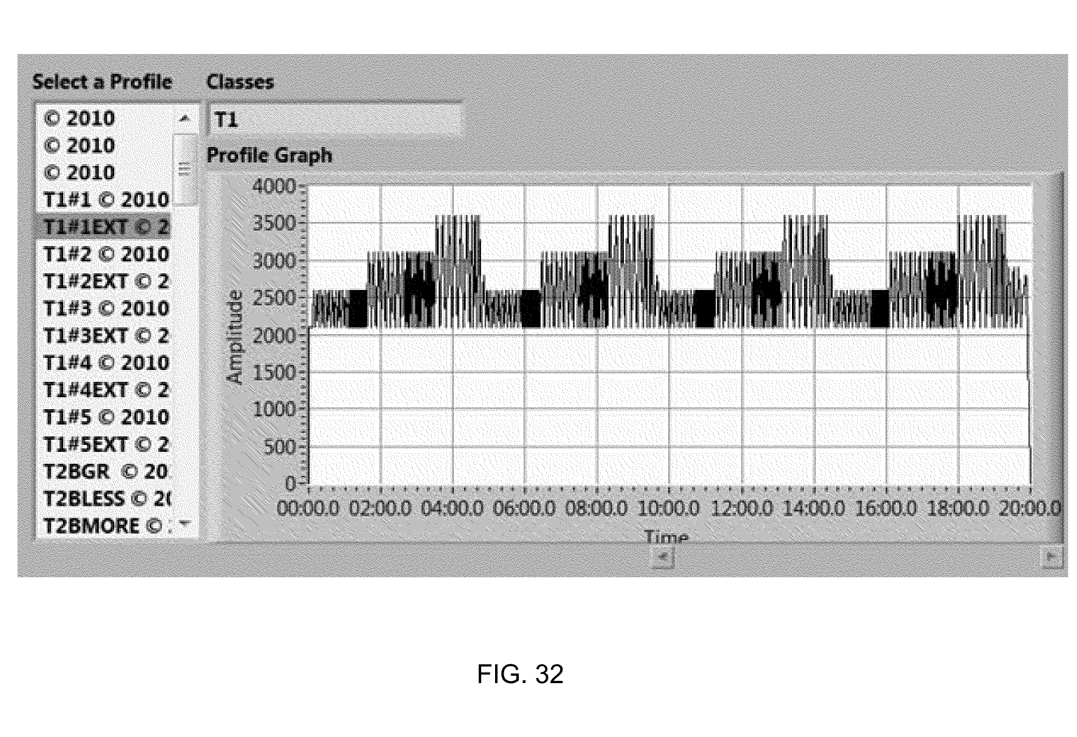

In some embodiments, a CVAC session includes a predetermined arrangement of pressure profiles to be administered to a user to acclimate a user to various profiles and or to prevent and/or reduce discomfort (e.g., in the ears) caused by pressure changes experienced by a user during a CVAC session. Examples of such CVAC sessions, each referred to as an “acclimatization CVAC session” herein, are shown in FIGS. 32-36. Such acclimatization CVAC sessions, like those shown in FIGS. 32-36, can be included in a set-up session or program and/or a treatment CVAC program for a user, for example in a program for the treatment of TBI, as described herein. In some embodiments, such acclimatization CVAC sessions, like those shown in FIGS. 32-36 can be optionally included in a user's treatment program only after the user experiences ear discomfort.

In one acclimatization CVAC session, as shown in FIG. 32, the session includes a start point at ambient pressure, an end point at ambient pressure, and a plurality of pressure targets therebetween. The plurality of pressure targets executed between the start point and end point include pressure targets having a minimum pressure of 2,100 ft (above ambient pressure) and a maximum pressure target of 3,600 ft (above ambient pressure). The acclimatization CVAC session includes a plurality of successive time intervals (e.g., a first time interval, a second time interval, a third time interval, and a fourth time interval) between the transitions from the start point and to the end point. In some embodiments, as shown in FIG. 32, during the first time interval, the CVAC session is configured to execute a first plurality of pressure targets in which a maximum pressure target is no more than 500 ft greater than a minimum pressure target, a second plurality of pressure targets in which a maximum pressure target is no more than 1,000 ft greater than the minimum pressure target, and a third plurality of pressure targets in which a maximum pressure target is no more than 1,500 ft greater than the minimum pressure target. For example, as shown in FIG. 32, during a first time interval of the CVAC session, a first plurality of targets are executed between 2,100 ft and 2,600 ft for a first time period, a second plurality of targets are executed between 2,100 ft and 3,100 ft for a second time period, and a third plurality of targets are executed between 2,100 ft and 3,600 ft. The pressure profile executed during the first time interval can be repeated during subsequent time intervals, as shown in FIG. 32. As shown in FIG. 32, the CVAC session can include a plurality time intervals each being up to approximately five minutes in duration.

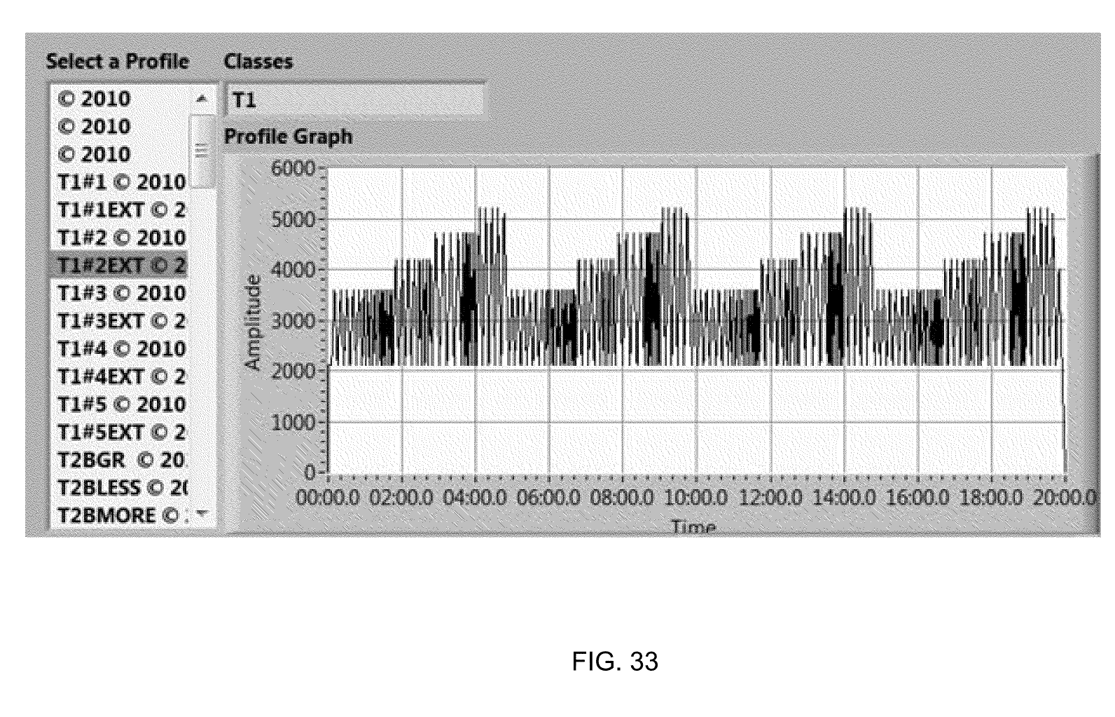

As shown in FIG. 33, in one acclimatization CVAC session, the session includes a start point at ambient pressure, an end point at ambient pressure, and a plurality of pressure targets therebetween. The plurality of pressure targets executed between the start point and end point include pressure targets having a minimum pressure of 2,100 ft (above ambient pressure) and a maximum pressure target of 5,200 ft (above ambient pressure). The acclimatization CVAC session includes a plurality of successive time intervals (e.g., a first time interval, a second time interval, a third time interval, and a fourth time interval) between the transitions from the start point and to the end point. In some embodiments, as shown in FIG. 33, during the first time interval, the CVAC session is configured to execute a first plurality of pressure targets in which a maximum pressure target is about 1,500 ft greater than a minimum pressure target, a second plurality of pressure targets in which a maximum pressure target is about 2,100 ft greater than the minimum pressure target, a third plurality of pressure targets in which a maximum pressure target is about 2,600 ft greater than the minimum pressure target, and a fourth plurality of pressure targets in which a maximum pressure target is about 3,100 ft greater than the minimum pressure target. For example, as shown in FIG. 33, during a first time interval of the CVAC session, a first plurality of targets are executed between 2,100 ft and 3,600 ft for a first time period, a second plurality of targets are executed between 2,100 ft and 4,200 ft for a second time period after the first time period, a third plurality of targets are executed between 2,100 ft and 4,700 ft for a third time period after the second time period, and a fourth plurality of targets are executed between 2,100 ft and 5,200 ft for a fourth time period after the third time period. The pressure profile executed during the first time interval can be repeated during subsequent time intervals, as shown in FIG. 33. As shown in FIG. 33, the CVAC session can include a plurality time intervals each being approximately five minutes in duration.

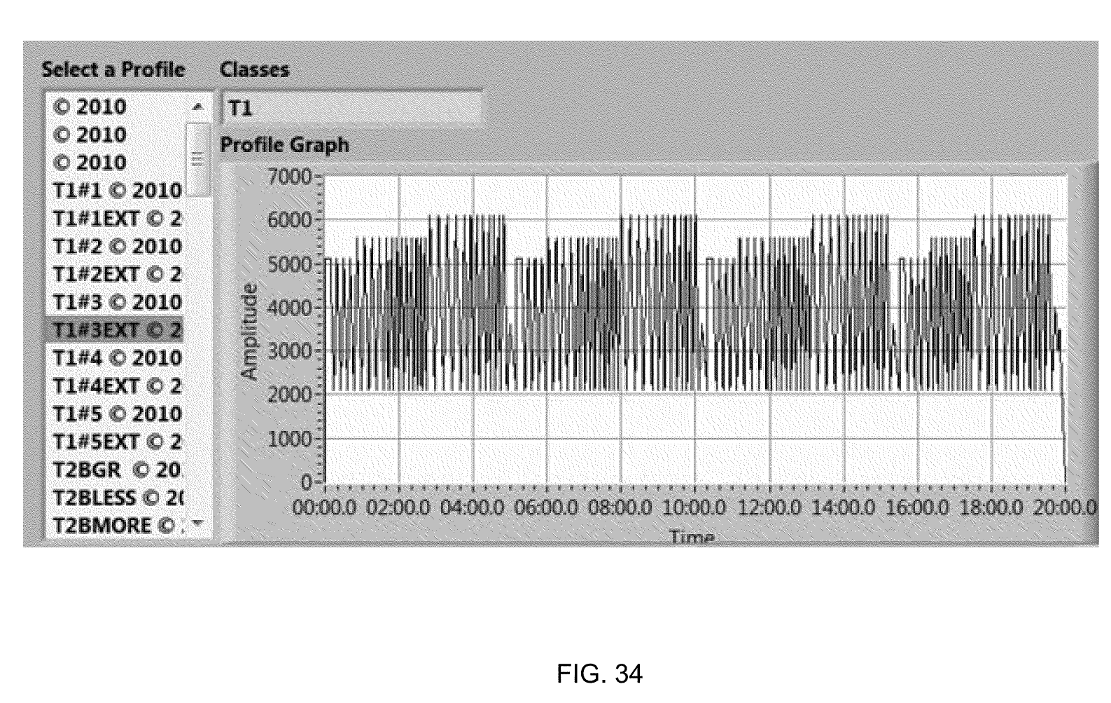

As shown in FIG. 34, in one acclimatization CVAC session, the session includes a start point at ambient pressure, an end point at ambient pressure, and a plurality of pressure targets therebetween. The plurality of pressure targets executed between the start point and end point include pressure targets having a minimum pressure of 2,100 ft (above ambient pressure) and a maximum pressure target of 6,100 ft (above ambient pressure). The acclimatization CVAC session includes a plurality of successive time intervals (e.g., a first time interval, a second time interval, a third time interval, and a fourth time interval) between the transitions from the start point and to the end point. In some embodiments, as shown in FIG. 34, during the first time interval, the CVAC session is configured to execute a first plurality of pressure targets in which a maximum pressure target is about 5000 ft greater than a minimum pressure target, a second plurality of pressure targets in which a maximum pressure target is about 5,500 ft greater than the minimum pressure target, and a third plurality of pressure targets in which a maximum pressure target is about 6,100 ft greater than the minimum pressure target. For example, as shown in FIG. 34, during a first time interval of the CVAC session, a first plurality of targets are executed between 2,100 ft and 5,100 ft for a first time period, a second plurality of targets are executed between 2,100 ft and 5,600 ft for a second time period after the first time period, and a third plurality of targets are executed between 2,100 ft and 6,100 ft for a third time period after the second time period. The pressure profile executed during the first time interval can be repeated during subsequent time intervals, as shown in FIG. 34. As shown in FIG. 34, the CVAC session can include a plurality time intervals each being up to approximately five minutes in duration.

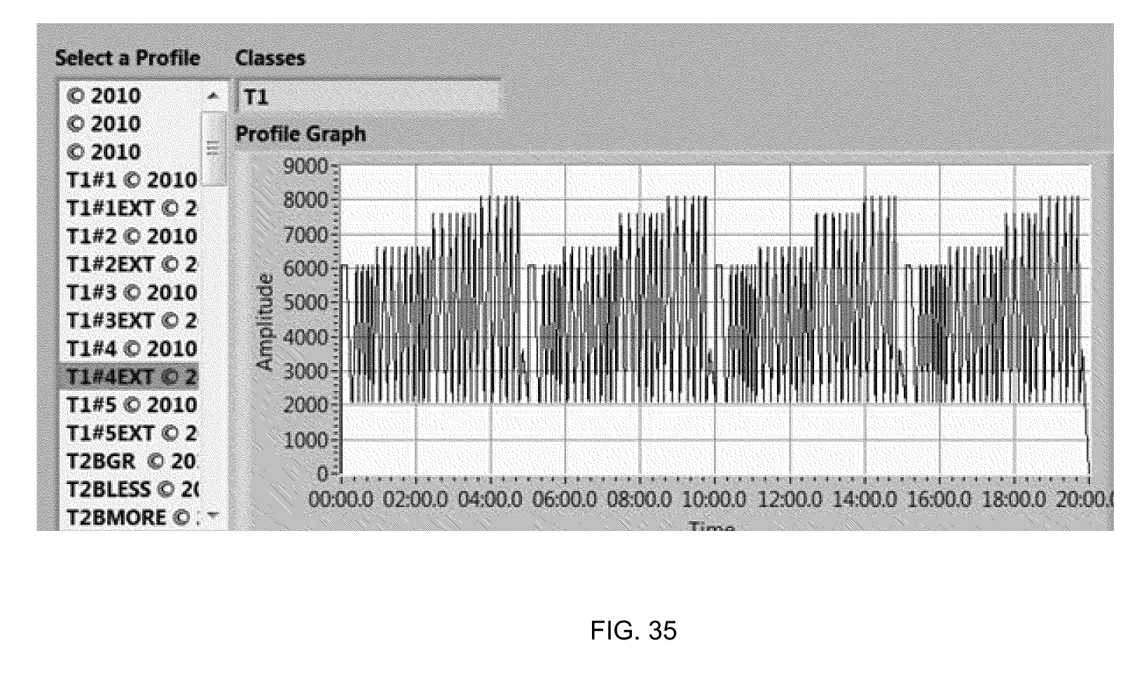

As shown in FIG. 35, in one acclimatization CVAC session, the session includes a start point at ambient pressure, an end point at ambient pressure, and a plurality of pressure targets therebetween. The plurality of pressure targets executed between the start point and end point include pressure targets having a minimum pressure of 2,100 ft (above ambient pressure) and a maximum pressure target of 8,100 ft (above ambient pressure). The acclimatization CVAC session includes a plurality of successive time intervals (e.g., a first time interval, a second time interval, a third time interval, and a fourth time interval) between the transitions from the start point and to the end point. In some embodiments, as shown in FIG. 35, during the first time interval, the CVAC session is configured to execute a first plurality of pressure targets in which a maximum pressure target is about 4,000 ft greater than a minimum pressure target, a second plurality of pressure targets in which a maximum pressure target is about 4,500 ft greater than the minimum pressure target, a third plurality of pressure targets in which a maximum pressure target is about 5,500 ft greater than the minimum pressure target, and a fourth plurality of pressure targets in which a maximum pressure target is about 6,000 ft greater than the minimum pressure target. For example, as shown in FIG. 35, during a first time interval of the CVAC session, a first plurality of targets are executed between 2,100 ft and 6,100 ft for a first time period, a second plurality of targets are executed between 2,100 ft and 6,600 ft for a second time period after the first time period, a third plurality of targets are executed between 2,100 ft and 7,600 ft for a third time period after the second time period, and a fourth plurality of targets are executed between 2,100 ft and 8,100 ft for a fourth time period after the third time period. The pressure profile executed during the first time interval can be repeated during subsequent time intervals, as shown in FIG. 35. As shown in FIG. 35, the CVAC session can include a plurality time intervals each being up to approximately five minutes in duration.

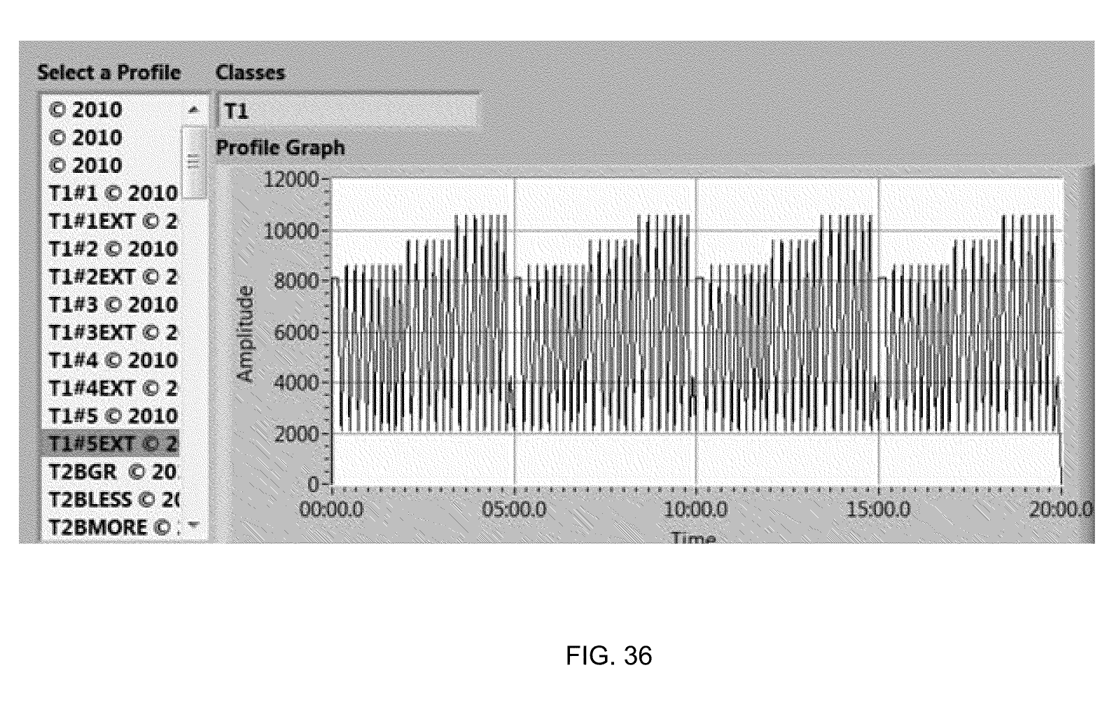

As shown in FIG. 36, in one acclimatization CVAC session, the session includes a start point at ambient pressure, an end point at ambient pressure, and a plurality of pressure targets therebetween. The plurality of pressure targets executed between the start point and end point include pressure targets having a minimum pressure of 2,100 ft (above ambient pressure) and a maximum pressure target of 10,600 ft (above ambient pressure). The acclimatization CVAC session includes a plurality of successive time intervals (e.g., a first time interval, a second time interval, a third time interval, and a fourth time interval) between the transitions from the start point and to the end point. In some embodiments, as shown in FIG. 35, during the first time interval, the CVAC session is configured to execute a first plurality of pressure targets in which a maximum pressure target is about 6,000 ft greater than a minimum pressure target, a second plurality of pressure targets in which a maximum pressure target is about 6,500 ft greater than the minimum pressure target, a third plurality of pressure targets in which a maximum pressure target is about 7,000 ft greater than the minimum pressure target, and a fourth plurality of pressure targets in which a maximum pressure target is about 8,500 ft greater than the minimum pressure target. For example, as shown in FIG. 36, during a first time interval of the CVAC session, a first plurality of targets are executed between 2,100 ft and 8,100 ft for a first time period, a second plurality of targets are executed between 2,100 ft and 8,600 ft for a second time period after the first time period, a third plurality of targets are executed between 2,100 ft and 9,600 ft for a third time period after the second time period, and a fourth plurality of targets are executed between 2,100 ft and 10,600 ft for a fourth time period after the third time period. The pressure profile executed during the first time interval can be repeated during subsequent time intervals, as shown in FIG. 36. As shown in FIG. 36, the CVAC session can include a plurality time intervals each being up to approximately five minutes in duration.

As shown in FIGS. 32-36, a CVAC session can be configured to acclimatize a user to pressure changes to alleviate discomfort associated with the pressure changes (e.g., ear discomfort) during a subsequent CVAC session by executing a repeating pattern of pressure targets in which the maximum target increases in a step-like manner.

The Interrupt:

During any phase in a Session wherein a user desires to stop the Session at that point for a short time, they may do so by activating an icon or other appropriate device on the on-board interface touch screen or control pad or notifying the operator of the device. This will hold the Session at the stage of interruption for a predetermined time period, such as a minute, at which time the Session will continue automatically. In some embodiments, a Session may be interrupted three times after which a staged descent will occur and the user will be required to exit the pressure vessel. The user's file may be flagged and the user may be placed back on the Set-Up Sessions until it is satisfactorily completed. A warning or reminder may be displayed on the screen each time an interrupt is used that informs the user of how many times interrupt has been used and the consequences of further use.

During any session, be it a Set-Up session or other type of session, a staged descent is also available if the user develops ear or sinus discomfort or wishes to terminate the session for any reason. A staged descent is characterized by slow, 1000 ft sine wave descent transits with re-ascensions of 500 ft at each step. The descents can be of greater or lesser transits but the ratio is usually about 1.5:1. At any time during the staged descent, the user can interrupt the descent and hold a given level or resume a previous level until comfort is achieved. The user may also re-ascend at their option if the staged descent is too aggressive. Any re-ascension is done in stages as described above. The user can subsequently indicate a “continue” on the descent and the staging will resume. This stepping continues until ambient pressure is reached whereupon the canopy or entrance to the device opens such that the user can exit the pressure vessel.

The Abort:

When a user wishes to end a session immediately and quickly exit the pressure vessel, the abort function can be activated. Touching the “abort” icon on the on-board interface touch pad/screen or notifying the operator of the device enables this option. A secondary prompt is activated acknowledging the command and asking the user if they are sure they want to abort. The user indicates their commitment to the command by pressing “continue” or “yes”. The program is aborted and a linear moderate descent is accomplished to ambient pressure whereupon the canopy or entrance to the device opens and the user exits. The user's file is flagged. The next time the user comes in for their session, the user is asked whether the abort was caused by discomfort. If yes, the user is placed back on the Set-Up session program. If no, the user is asked if they wish to resume their regularly scheduled session. The client is given the option of resuming their regularly scheduled Session or returning to the Set-Up session.

Program and Target Criteria, Including Medically Significant Criteria:

In some embodiments, a user is categorized into a group of users having similar body-types with similar characteristics based upon answers to a questionnaire or information otherwise obtained from the user. The information from the user guides the construction of custom CVAC programs for each individual. When administering CVAC programs for treatment of chronic pain, the medical status of the user can also be used to determine appropriate pressures and additional parameters (such as duration, temperature, or humidity) of the targets. Custom session targets may be administered based upon the medical condition and therapy desired. The acceptable and appropriate target parameters may be obtained as described herein and through consultation with the user's physician or other appropriate health-care provider prior to designing session targets and administering a CVAC session. However the known contraindications of CVAC are similar to those of commercial air travel, allowing for a broad range of application.

Hypoxic Conditioning:

Initial understanding in the art about the effects of hypoxia focused on increased oxygenation of the blood via increased production of red blood cells mediated by increases in EPO production. While increases in EPO production are believed to increase red blood cell production, its effects are not limited to this activity. Molecules such as HIF, induced by hypoxia, regulate EPO production in addition to a variety of other activities including metabolism, angiogenesis, and vascular tone—the stimulation of which may all play a role in protecting tissue from subsequent hypoxic damage. This protection may occur prophylactically, post-ischemic or traumatic events as well as facilitating stem cell mobilization and red blood cell production. [Eckardt K. U., Kurtz, A., Regulation of erythropoietin production, Eur. J. Clin. Invest., 35(Supp. 3):13-19, (2005)]. Attempts to improve blood donation volume and frequency have focused on the administration of erythropoietin. Erythropoietin is known to induce red blood cell production, thus increasing red blood cell volume in the patient. [Kirsh K A, et al., Erythropoietin as a volume-regulating hormone: an integrated view. Semin. Nephrol., 25(6):388-91 (2005)]. Recent research demonstrated the dramatic increase in red blood cell volume in children following the administration of erythropoietin. This increase allowed for autologous donation by the children of the study prior to undergoing open-heart surgery. The increase in red blood cell volume prevented drops in red blood cell volumes typically associated with blood donation, especially in children. The maintenance of stable red blood cell counts despite repeated donations in a 20 day period allowed for autologous donation of sufficient blood volumes in anticipation of each child's surgery as well as maintained sufficient blood counts to allow for subsequent surgery. [Sonzogni V, et al., Erythropoietin therapy and preoperative autologous blood donation in children undergoing open heart surgery, Brit. J. Anaesth., 87(3):429-34 (2001)]. Additional studies have also demonstrated the effectiveness of erythropoietin in improving red blood cell volumes, donation volumes, and ability to donate multiple times. [Goodnough L T, et al., Preoperative red cell production in patients undergoing aggressive autologous blood phlebotomy with and without erythropoietin therapy, Transfusion, 32(5):441-5 (1992); Biesma D H, et al., The efficacy of subcutaneous recombinant human erythropoietin in the correction of phlebotomy-induced anemia in autologous blood donors, Transfusion 33(10):825-9 (1993)].

Moderate static hypoxic preconditioning is known to provide protection from tissue and cellular damage via tolerance. When the environmental oxygen levels are reduced (hypoxia), downstream effects include protection from damage due to subsequent hypoxia. This tolerance is not yet completely understood, but it has been linked to various cellular mechanisms and molecules, including, but not limited to, molecules such as erythropoietin (EPO), hypoxia-inducible factor (HIF), Tumor Necrosis Factor (TNF), glycogen, lactate, and others. [Sharp, F., et al., Hypoxic Preconditioning Protects against Ischermic Brain Injury, NeuroRx: J. Am. Soc. Exp. Neuro., Vol. 1: 26-25 (2004)]. Additionally, beneficial static hypoxic conditioning is not purely additive. Administration of sequential sessions can have detrimental effects. Oxygen concentrations that are too low result in detrimental effects to the tissues as well as the entire body. Similarly, hypoxia conditioning of longer durations can have detrimental effects in addition to providing some desired beneficial effects. [Sharp, F., et al., Hypoxic Preconditioning Protects against Ischermic Brain Injury, NeuroRx: J. Am. Soc. Exp. Neuro., Vol. 1: 26-25 (2004)]. Furthermore, prior hypoxic conditioning studies utilized static pressures over lengthy time-frames. In contrast to the aforementioned static hypoxic conditioning known in the art, CVAC sessions utilize multiple variations in altitudes and variable, time-frames of application. The combination of varying pressures over varying time frames, including rapid changes over varying time-frames, produces multiple beneficial effects associated with hypoxic condition, stimulates additional beneficial effects, and does not result in the detrimental effects seen with static hypoxic conditioning. Similarly, the duration of CVAC sessions, while not limited, are typically much shorter than the long blocks of time currently used for static hypobaric conditioning. Thus, the use of unique CVAC sessions for the production of beneficial hypoxic effects provides a novel and superior alternative to the current methods of static hypoxic conditioning as described above.

Treatment Types:

In some embodiments of the present invention, CVAC Program is used to treat users who wish to increase their production of blood. CVAC is administered to increase the oxygenation of the blood, increase the number of red blood cells within a user, increase the production of HIF's, and/or stimulate other associated physiological processes affected by CVAC treatment such as fluid (lymph, blood, or other bodily fluids) movement. Treatment is administered through the use of one or more CVAC sessions. Such sessions may be user defined or custom-defined with input from the user's physician.

Modulating, in the context of assessment of CVAC sessions, has multiple meanings. In the context of blood production, modulation means any changes that result in the increased numbers of red blood cells, hematocrit, or blood volume. Additionally, modulation in the context of stem cell therapy means increases in stem cell mobilization, reduction in recovery time compared to standard therapies, less painful recovery compared to standard therapies, and/or more robust responses in physiological parameters compared to standard therapies.

Attempts to improve stem cell mobilization, engraftment, and post-transplantation recovery have focused on the administration of erythropoietin. Erythropoietin (EPO) is known to induce red blood cell production, thus increasing red blood cell volume in the patient. [Kirsh K A, et al., Erythropoietin as a volume-regulating hormone: an integrated view, Semin. Nephrol., 25(6):388-91 (2005)]. Typical mobilization protocols utilize the cytokine granulocyte colony stimulating factor (G-CSF). However, the addition of EPO is also known to boost hematopoietic precursor cells (stem cells) as well as immune effector cells, thus improving the collection during mobilization and increasing the percentage of cells for successful engraftment. [Joshi et al., Immunological properties of mononuclear cells from blood stem cell harvests following mobilization with erythropoietin+G-CSF in cancer patients” Cytotherapy 2(1):15-24 (2002)]. A final mobilization factor is the cytokine vascular endothelial growth factor (VEGF). In additional to stimulating angiogenesis, VEGF has been linked with increased mobilization of stem cells from the bone marrow, thus providing another factor for improving pre-transplantation mobilization.

CVAC sessions maybe use for the treatment of inflammation or swelling or combinations thereof due to TBI. CVAC is administered to increase the oxygenation of the inflamed or swollen tissue, increase the production of HIF's, and stimulate other associated physiological processes affected by CVAC treatment such as fluid (lymph, blood, or other bodily fluids) movement and reduction in swelling. Treatment is administered through the use of one or more CVAC sessions. Such sessions may be user defined or custom-defined with input from the user's physician. CVAC sessions may be administered in advance of any surgeries or other treatment regimens to help reduce or prevent any damaging effects relating to inflammation and swelling.

CVAC sessions may be used to treat a wide variety of ischemia. As defined herein, treatment of ischemia includes prevention of ischemia, treatment of ischemia, prophylactic treatment of ischemia, amelioration of ischemia, as well as recovery from an ischemic event. In one embodiment of the present invention, at least one CVAC session is used to prophylactically treat users who are at risk for TBI or cerebral ischemia (strokes). A stroke is the acute neurological injury caused by any one of a variety of pathologic processes involving the blood vessels of the brain. Such processes may include occlusion of vessels, known weaknesses in vessel walls, inadequate cerebral flow, and rupture of cerebral vessels. Diagnosis of predisposal for stroke can be accomplished by any means commonly used in the medical community or by one of ordinary skill in the art.

CVAC sessions may be used to treat users who have Alzheimer's disease and/or symptoms of the disease. CVAC is administered to increase the oxygenation of the affected tissue (e.g. the brain), increase the production of HIFs, and/or stimulate other associated physiological processes affected by CVAC treatment such as fluid (lymph, blood, cerebral, spinal, or other bodily fluids) movement.

CVAC sessions for any of the aforementioned aspects and embodiments may also be used in combination with pharmaceutical regimens or non-pharmaceutical therapies such as physical therapy or homeopathic therapies. As described above, CVAC sessions of any combination or permutation can be administered prior to, concurrent with, or subsequent to administration of a pharmaceutical, pharmaceuticals, or non-pharmaceutical therapy. Myriad permutations of pharmaceutical therapies, non-pharmaceutical therapies, and CVAC session combinations are possible, and combinations appropriate for the type of medical condition and specific pharmaceutical may be identified with the help of any person skilled in the art, such as a treating physician.

Methods of Treatment:

CVAC treats many of the condition associated with Traumatic brain injury (TBI). CVAC may be administered after a TBI event to limit the injury to the brain through the use of one or more CVAC sessions. Such sessions may be user defined or custom-defined with input from the user's physician. A further embodiment of the invention includes the use of CVAC sessions when TBI is anticipated. CVAC sessions may be administered prior to TBI to lessen the potential brain tissue injury that may occur. More specifically, in some embodiments, one or more CVAC sessions can be administered to the user, for example if the user has a high likelihood or risk of experiencing a TBI event (e.g., an athlete, a member of the military, or the like), before such a TBI event occurs to improve certain physiological parameters of the user (e.g., to increase mitochondria in the brain) thus reducing the effect and/or severity of injury from a subsequent TBI event.

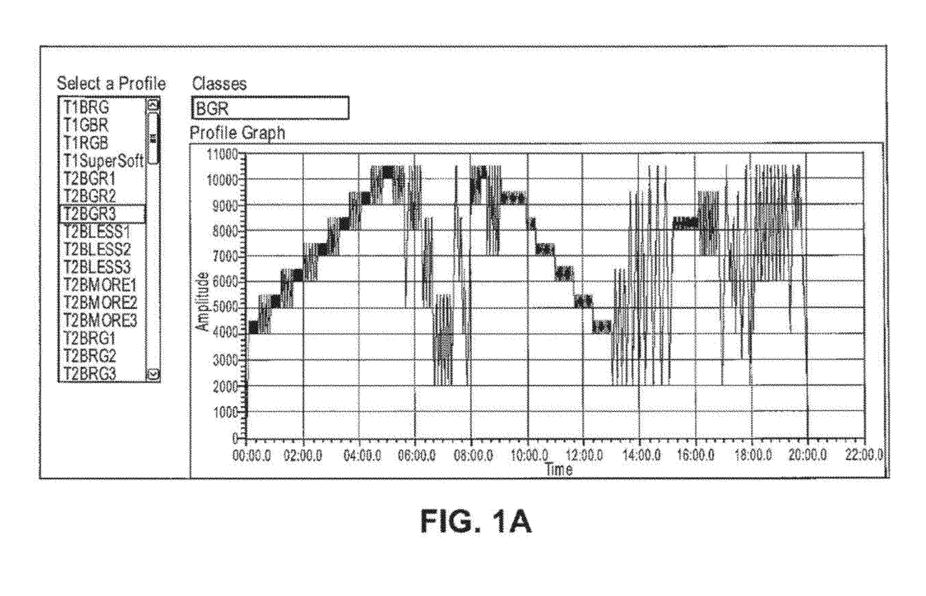

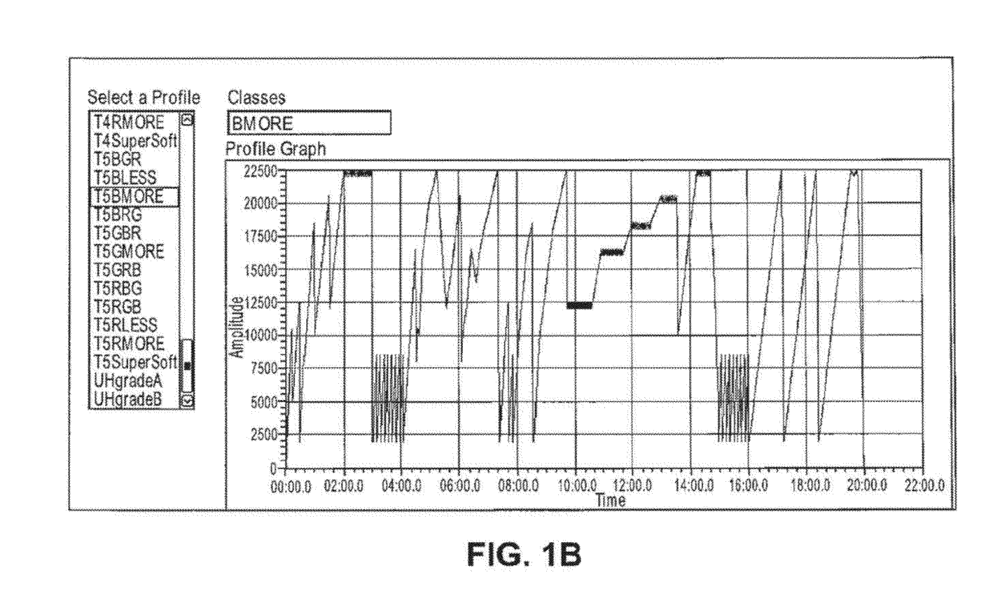

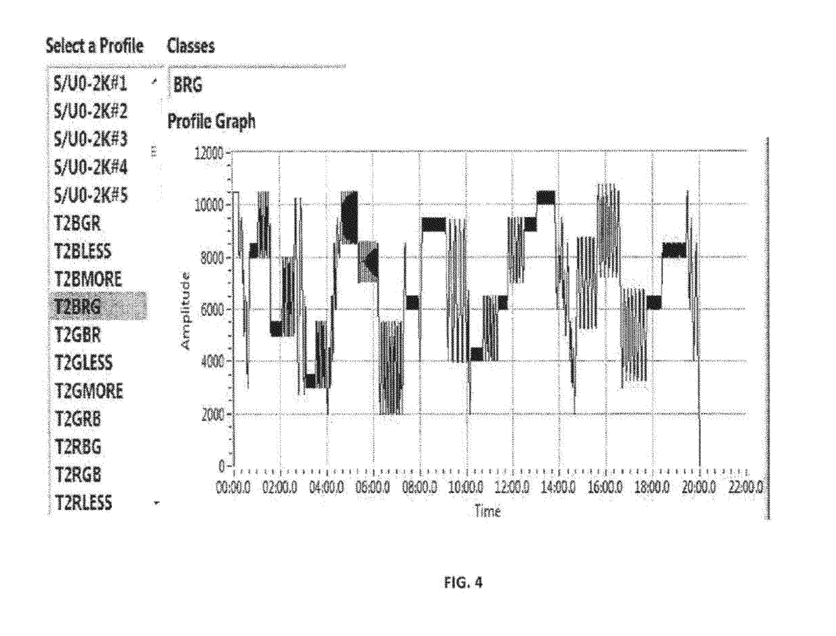

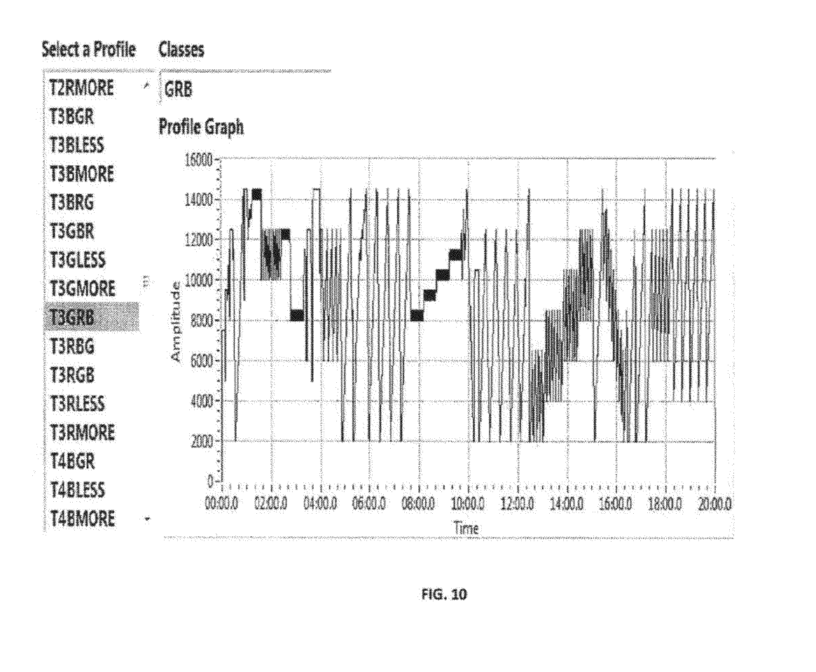

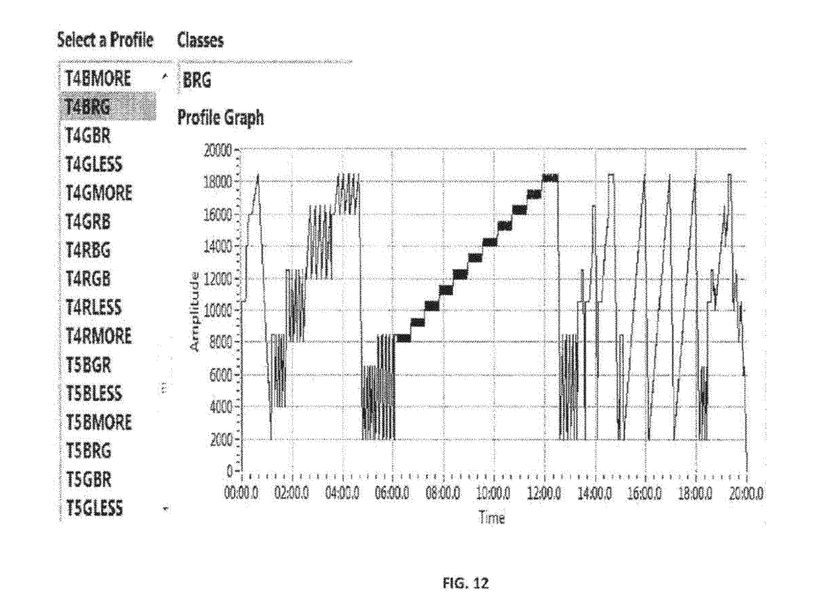

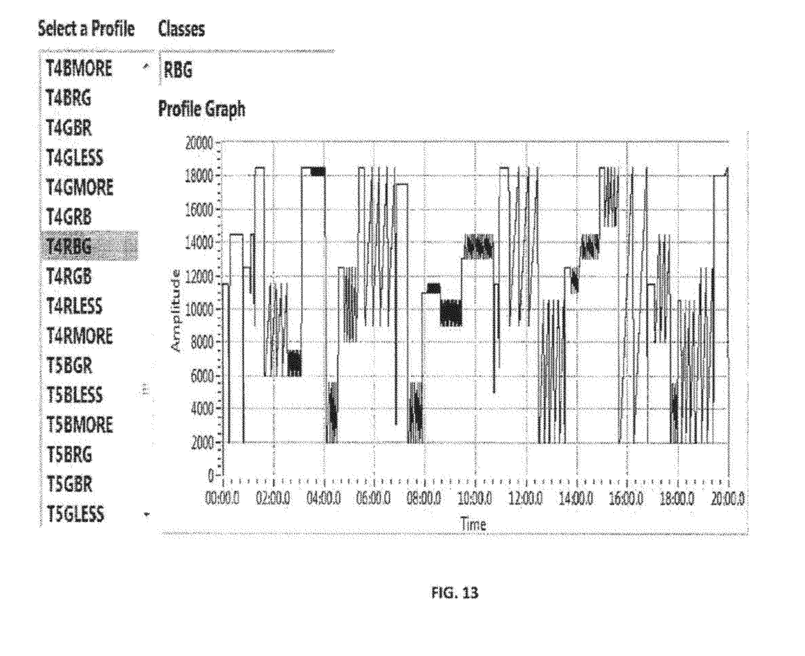

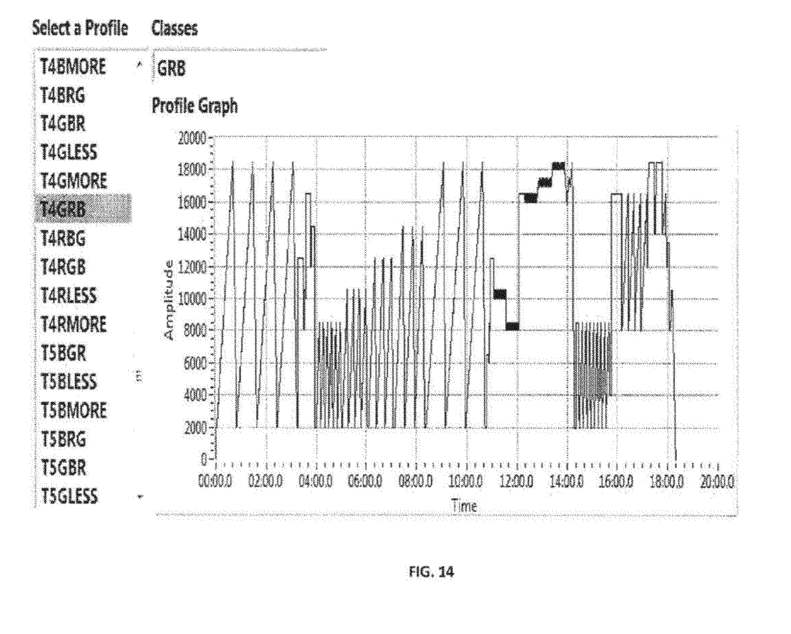

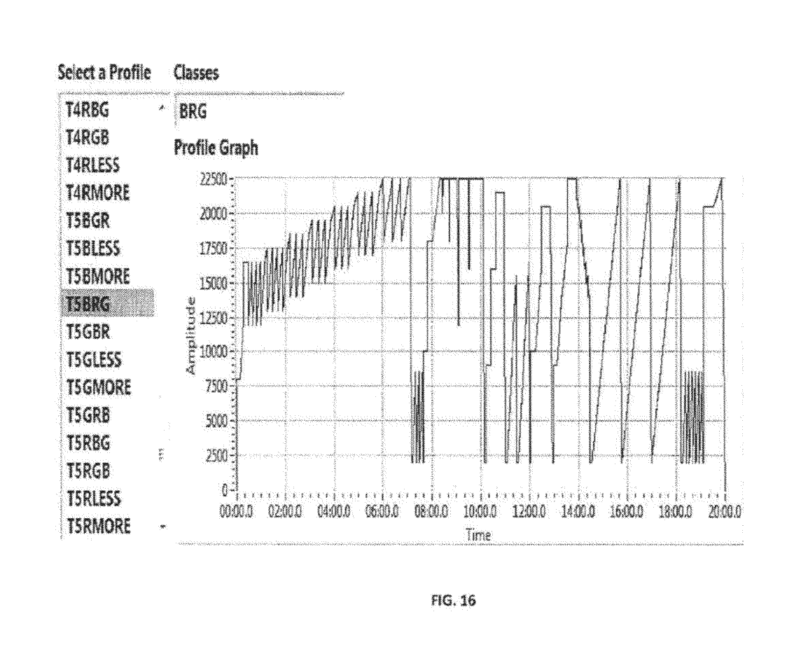

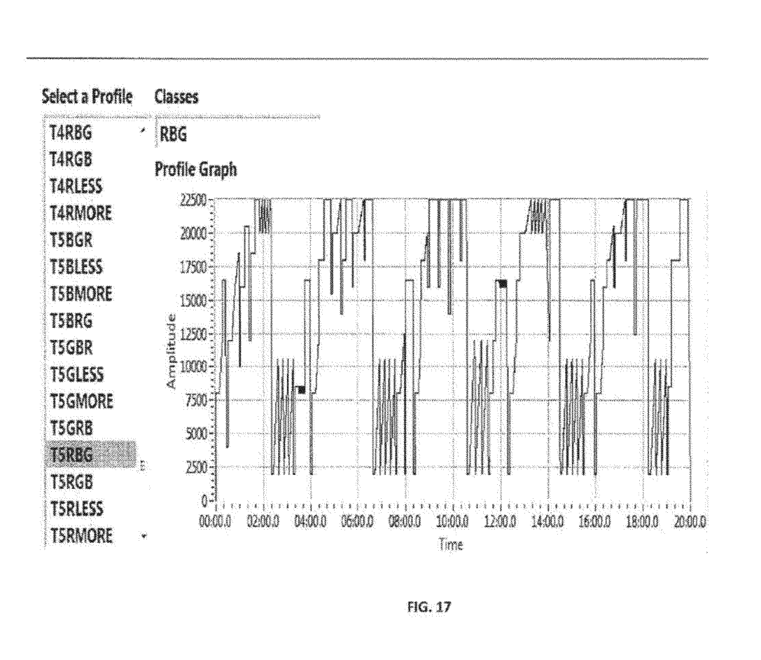

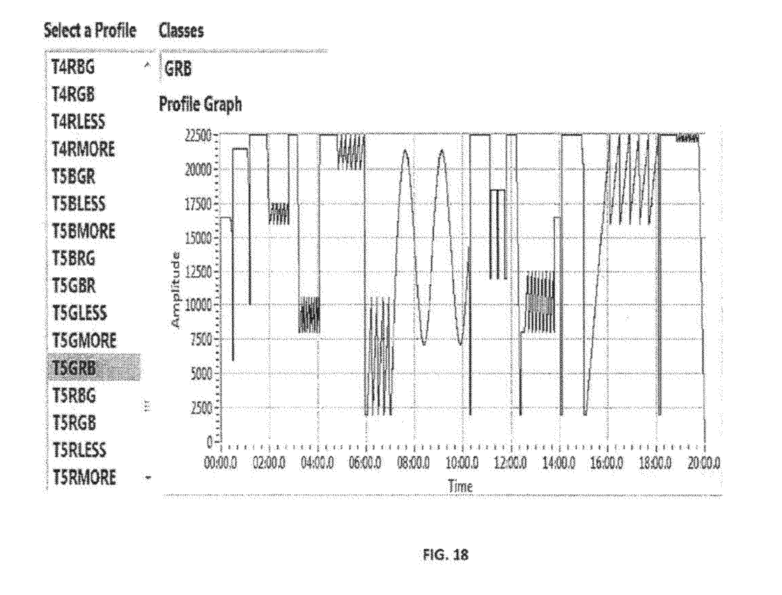

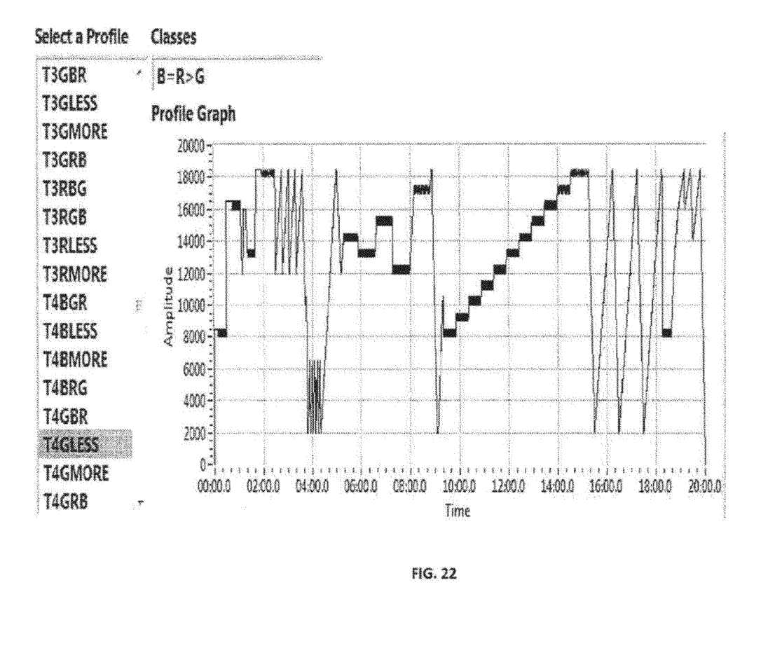

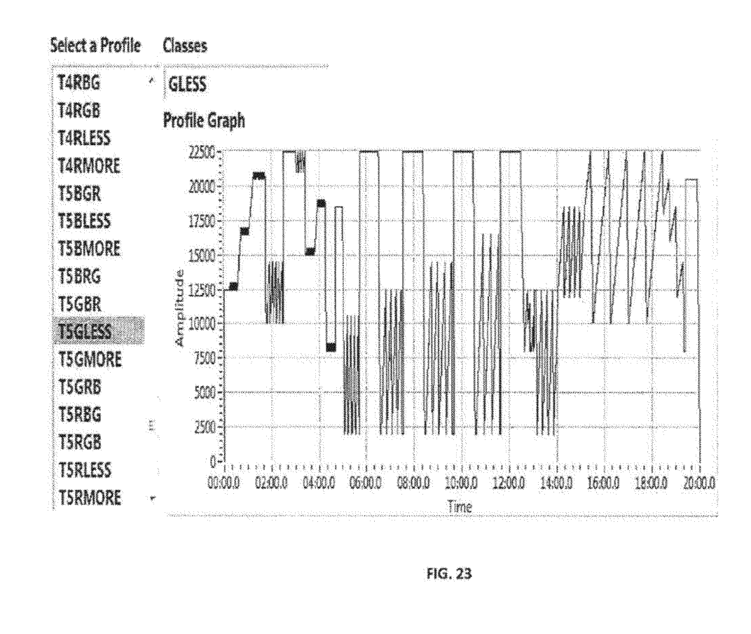

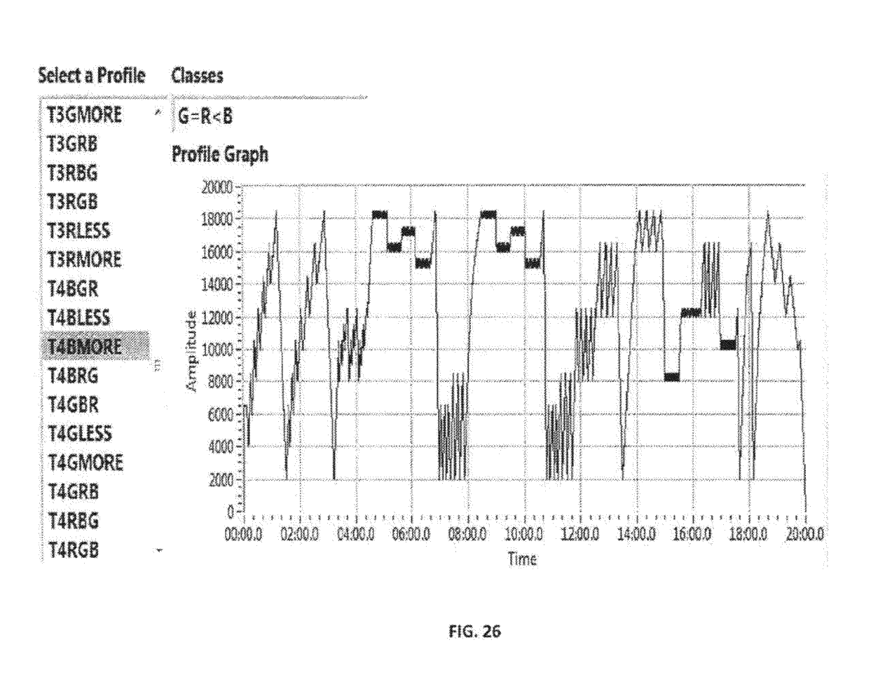

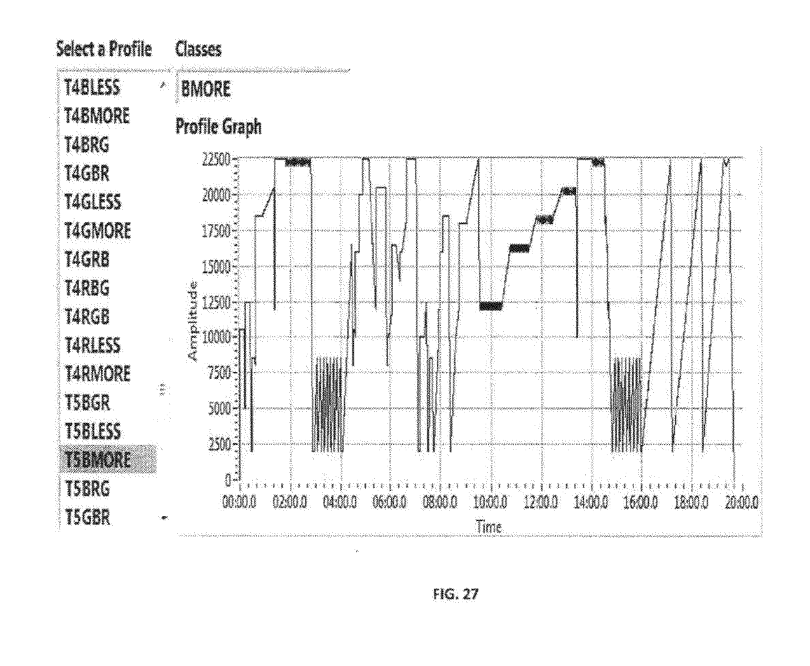

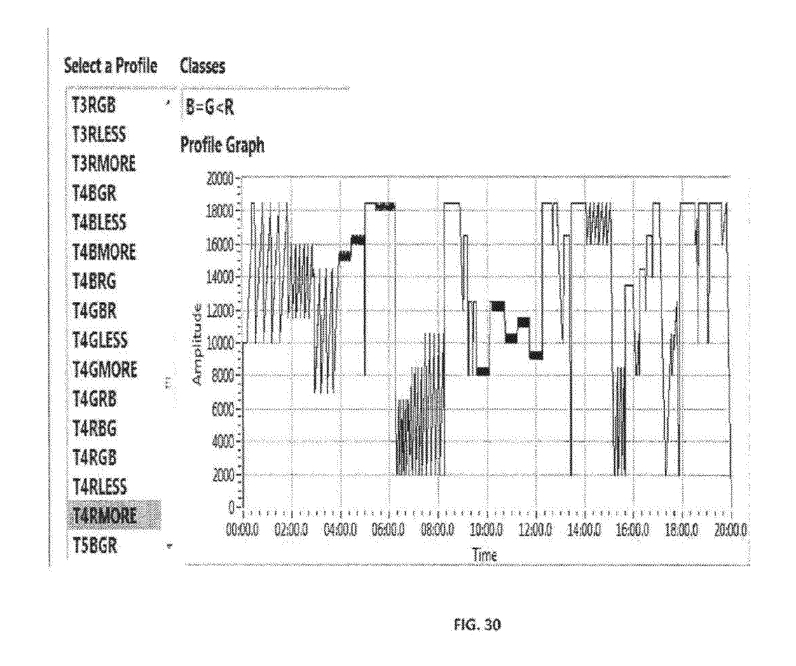

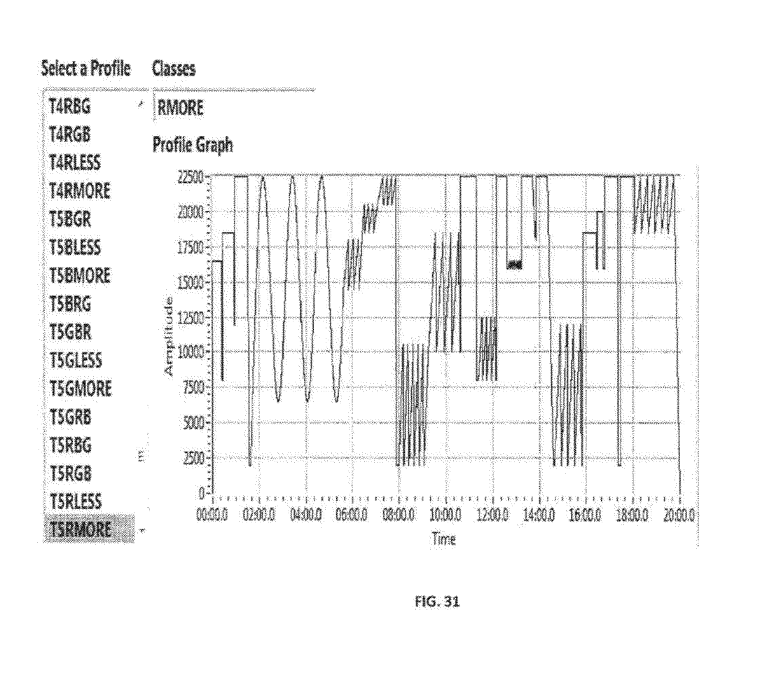

Specific examples of a CVAC session are shown graphically in FIGS. 1A and 1B. In both figures, the parameters of the program are shown as a line graph with axes that correspond to time (x-axis) and pressure change (y-axis). The pressure change is shown in amplitudes, and corresponds to a pressure at a number of feet above atmospheric pressure (represented by the number zero). Examples of a CVAC session suitable administration to a user for the treatment and/or prevention of TBI are also shown in FIGS. 4-6, 8-10, 12-14, 16-18, 20-43.

In some embodiments, CVAC sessions for the treatment of TBI are administered for at least 10 minutes, and in some embodiments for at least 20 minutes, with variable frequency. Additional administration periods may include, but are not limited to, about 10 minutes, about 20 minutes, about 30 minutes, about 40 minutes, about 60 minutes, between 10 and 20 minutes, between 20 and 30 minutes, between 30 and 60 minutes, and between 60 and 120 minutes.

Frequencies of sessions or series of sessions may include, but are not limited to, daily, monthly, or when medically indicated or prescribed. The frequency and duration of the sessions can be altered to suit the medical condition to be treated, and CVAC sessions may be administered as single sessions, or as a series of sessions, preferably with a Set-Up Session as described herein. For example, the frequency of sessions or series of sessions can be administered 3 times a week for 8 weeks, 4 times a week for 8 weeks, 5 times a week for 8 weeks, or 6 times a week for 8 weeks. Additional frequencies can be easily created for each individual user.

Similarly, the targets in the sessions can also be altered or adjusted to suit the individual and medical condition to be treated. If at any time the user or attendant determines that the session is not being tolerated well, an abort may be initiated and the user brought down safely and exited. The permutations of targets can be customized to the individual, and may again be identified with the help of any person skilled in the art, such as a treating physician. Furthermore, the variations may be administered in regular intervals and sequence, as described, or in random intervals and sequence. The variations in number, frequency, and duration of targets and sessions can be applied to all methods of treatment with CVAC described herein. Treat or treatment, as used herein refers to the treatment of TBI and includes, but is not limited to, inhibiting, arresting, relieving, or stopping TBI. Thus, as used herein, the term “treatment” is used synonymously with the terms “alleviation,” “amelioration,” “prophylaxis,” or “prevention.”

CVAC sessions for blood production and stem cell therapy, including but not limited to uses to aid in stem cell mobilization, stem cell engraftment, and recovery following transplantation, are administered preferably for at least 10 minutes, and more preferably at least 20 minutes, with variable frequency. CVAC sessions are administered preferably for at least 10 minutes, and more preferably at least 20 minutes, with variable frequency. Additional administration periods may include, but are not limited to, about 10 minutes, about 20 minutes, about 30 minutes, about 40 minutes, about 60 minutes, between 10 and 20 minutes, between 20 and 30 minutes, between 30 and 60 minutes, and between 60 and 120 minutes.

Frequencies of sessions or series of sessions may include, but are not limited to, daily, monthly, or when medically indicated or prescribed. The frequency and duration of the sessions can be altered to suit the medical condition to be treated, and CVAC sessions may be administered as single sessions, or as a series of sessions, preferably with a Set-Up Session as described herein. For example, the frequency of sessions or series of sessions can be administered 3 times a week for 8 weeks, 4 times a week for 8 weeks, 5 times a week for 8 weeks, or 6 times a week for 8 weeks. Additional frequencies can be easily created for each individual user. Similarly, the targets in the sessions can also be altered or adjusted to suit the individual and medical condition to be treated. The permutations of targets can be customized to the individual, and may again be identified with the help of any person skilled in the art, such as a treating physician. Furthermore, the variations may be administered in regular intervals and sequence, as described, or in random intervals and sequence. The variations in number, frequency, and duration of targets and sessions can be applied to all methods of treatment with CVAC described herein.

Efficacy of Treatment

Erythropoiesis

Efficacy of CVAC treatments for red blood cell production can be evaluated with a variety of imaging and assessment techniques known in the art. Assessment criteria known in the art include: hematocrit measurement, blood-gas analysis, extent of blood-perfusion of tissues, angiogenesis within tissues, erythropoietin production, and recovery of blood volume and red blood cell counts. Additional criteria for assessing the production of red blood cells may be known by those of skill in the art and can be employed to assess the beneficial effects of CVAC programs.

By example only, modulation of hematocrit is indicative of CVAC efficacy for red blood cell production. Conversely, a lack of change in the user's hematocrit (or with any of the physiological markers described herein) does not necessarily indicate that the CVAC treatments are not achieving positive results. Angiogenesis within affected tissues can also be a physiological marker used to assess CVAC efficacy. Modulation of vessel development within the tissues or body of a user during or following one or more CVAC sessions is indicative of efficacious CVAC treatments. Again, by example only, angiogenesis may be assessed by a variety of imaging and detection methods including dyes, MRI, fluoroscopy, endoscopy, and other means known in the art. Additionally, initiation or modulation of VEGF expression within affected tissues during or following one or more CVAC sessions is also indicative of efficacious CVAC treatment. Modulation of erythropoietin production following one or more CVAC sessions is also a physiological marker used to assess the efficacy of CVAC treatments. In one embodiment of the present invention, increases in the expression of erythropoietin indicate efficacious CVAC treatments. Similarly, when blood-gas analysis is the physiological marker used to assess CVAC efficacy, modulation of the dissolved gasses in the blood during or following one or more CVAC sessions is indicative of efficacious CVAC treatment. Typical gasses monitored include oxygen, carbon dioxide, and nitrogen. However, any gas found within the blood may be monitored for assessment of CVAC efficacy. When blood-perfusion of the tissues is the physiological marker used to assess CVAC efficacy, increases in blood volumes or blood exchange and combinations thereof within tissues during or following one or more CVAC sessions are indicative of the efficacious CVAC treatment. Additional criteria for assessing the production of red blood cells may be known by those of skill in the art and can be employed to assess the beneficial effects of CVAC programs.

Stem Cell Therapy

Efficacy of CVAC treatments for mobilization of stem cells, engraftment of stem cells, and recovery following stem cell therapy can be evaluated with a variety of imaging and assessment techniques known in the art. Assessment criteria known in the art include, but are not limited to: assessment of EPO levels, assessment of VEGF levels, assessment of cytokine profiles, peripheral blood stem cell counts, peripheral blood immune effector cell counts, hematocrit measurement, blood-gas analysis, extent of blood-perfusion of tissues, angiogenesis within tissues, and recovery of blood volume and red blood cell counts. Additional criteria for assessing the production of red blood cells may be known by those of skill in the art and can be employed to assess the beneficial effects of CVAC programs.

Modulation of stem cell counts in the peripheral blood, prior to and/or following mobilization, is indicative of efficacious CVAC treatments. Similarly, modulation of immune effector cell counts prior to and/or following mobilization is indicative of efficacious CVAC treatment. Modulation of hematocrit is indicative of CVAC efficacy for mobilization of stem cells, engraftment of stem cells, or recovery from stem cell therapy. Conversely, a lack of change in the user's hematocrit (or with any of the physiological markers described herein) does not necessarily indicate that the CVAC treatments are not achieving positive results. Angiogenesis within affected tissues can also be a physiological marker used to assess CVAC efficacy. Modulation of vessel development within the tissues or body of a user during or following one or more CVAC sessions is indicative of efficacious CVAC treatments. Again, by example only, angiogenesis may be assessed by a variety of imaging and detection methods including dyes, MRI, fluoroscopy, endoscopy, and other means known in the art. Additionally, initiation or modulation of VEGF expression within affected tissues during or following one or more CVAC sessions is also indicative of efficacious CVAC treatment. Modulation of EPO production following one or more CVAC sessions is also a physiological marker used to assess the efficacy of CVAC treatments. In one embodiment of the present invention, increases in the expression of EPO indicate efficacious CVAC treatments. Similarly, when blood-gas analysis is the physiological marker used to assess CVAC efficacy, modulation of the dissolved gasses in the blood during or following one or more CVAC sessions is indicative of efficacious CVAC treatment. Typical gasses monitored include oxygen, carbon dioxide, and nitrogen. However, any gas found within the blood may be monitored for assessment of CVAC efficacy. When blood-perfusion of the tissues is the physiological marker used to assess CVAC efficacy, increases in blood volumes or blood exchange and combinations thereof within tissues during or following one or more CVAC sessions are indicative of the efficacious CVAC treatment.

Engraftment and recovery following transplantation can also be assessed utilizing any of the methods detailed above. By way of example, flow cytometry for the determination of Mean Fluorescence Index (MFI) or Mean Reticulocyte Volume (MRV) can be utilized to assess CVAC efficacy related to engraftment following transplantation. Similarly, complete blood counts can be performed to assess recovery following transplantation therapy. Additional criteria for assessing the mobilization of stem cells, engraftment of stem cells, and recovery following stem cell therapy may be known by those of skill in the art and can be employed to assess the beneficial effects of CVAC programs.

Inflammation and Swelling

Efficacy of CVAC treatments for inflammation and swelling can be evaluated with a variety of imaging and assessment techniques known in the art. Examples include methods such as magnetic resonance imaging (MRI) of the affected region, invasive imaging through catheterization, or alternative non-invasive imaging methods. Additional criteria useful in assessing the efficacy of CVAC sessions include: hematocrit measurement, blood-gas analysis, extent of blood-perfusion of tissues, angiogenesis within tissues, erythropoietin production, extent of tissue necropsy in the affected tissues, and assessment of additional physical indicators such as reduction in swelling, temperature, and turgidity. Assessment of immune or inflammation-mediating cells present in the affect tissue, chemokine and cytokine profiles in the affected tissue, or other immune-cell factors can also aid in the evaluation of efficacy. Additional criteria for assessing the treatment and prevention inflammation and swelling may be known by those of skill in the art and can be employed to assess the initial or further beneficial effects of CVAC programs.

By example only, when hematocrit is the physiological marker used to assess CVAC efficacy, modulation of hematocrit during or following one or more CVAC sessions is indicative of efficacious CVAC treatment for the treatment, amelioration, or prevention of inflammation and swelling. In one embodiment, an increase in hematocrit is indicative of efficacious CVAC treatment. Conversely, a lack of change in the user's hematocrit (or with any of the physiological markers described herein) does not necessarily indicate that the CVAC treatments are not achieving positive results. Similarly, when blood-gas analysis is the physiological marker used to assess CVAC efficacy, modulation of the dissolved gasses in the blood during or following one or more CVAC sessions is indicative of efficacious CVAC treatment. Typical gasses monitored include oxygen, carbon dioxide, and nitrogen. However, any gas found within the blood may be monitored for assessment of CVAC efficacy. When blood perfusion of the tissues is the physiological marker used to assess CVAC efficacy, increases in blood volumes or blood exchange and combinations thereof within tissues during or following one or more CVAC sessions are indicative of the efficacious CVAC treatment. Angiogenesis within affected tissues can also be a physiological marker used to assess CVAC efficacy. Modulation of vessel development within the affected tissues during or following one or more CVAC sessions is indicative of efficacious CVAC treatments. Additionally, initiation or modulation of VEGF expression within affected tissues during or following one or more CVAC sessions is also indicative of efficacious CVAC treatment. Modulation of erythropoietin production following one or more CVAC sessions is also a physiological marker used to assess the efficacy of CVAC treatments. In one embodiment of the present invention, increases in the expression of erythropoietin indicate efficacious CVAC treatments.

Extent of tissue necropsy is a further physiological marker used to assess CVAC efficacy. Modulation of tissue necropsy, including repair or efficient removal of affected tissue by known bodily repair systems, pathways, and cascades as well as prevention of initial or continued necrosis, during or following one or more CVAC sessions is indicative of CVAC session efficacy. Still further physical indicators for assessing efficacy of CVAC sessions include modulation of swelling, temperature, or turgidity and combinations thereof during or following one or more CVAC sessions. In one embodiment, reduced swelling, temperature, or turgidity or combinations thereof are indicative of efficacious CVAC treatment. Similarly, in yet another embodiment modulation of immune or inflammation mediating cells present in the affected tissue, chemokine and cytokine profiles in the affected tissue, or other immune-cell factors or a combination thereof is also indicative of efficacious CVAC treatment. For example, cytokine profiles of interleukins within the affected tissues or body can be monitored to determine efficacy of CVAC treatments. Additional criteria for assessing the treatment and prevention of inflammation or swelling or a combination thereof may be known by those of skill in the art and can be employed to assess the beneficial effects of CVAC programs.

Ischemia

Efficacy of CVAC treatments for cardiac and cerebral ischemia can be evaluated with a variety of imaging and assessment techniques known in the art. Examples include methods such as magnetic resonance imaging (MRI) of the affected region, invasive imaging through catheterization, or alternative non-invasive imaging methods. Additional assessment criteria known in the art include: hematocrit measurement, blood-gas analysis, extent of blood-perfusion of tissues, angiogenesis within tissues, erythropoietin production, extent of tissue necropsy following ischemic events, and assessment of cognitive abilities and/or motor skills following ischemic events.

By example only, when hematocrit is the physiological marker used to assess CVAC efficacy, modulation of hematocrit during or following one or more CVAC sessions is indicative of efficacious CVAC treatment for the treatment, amelioration, or prevention of ischemic events. In one embodiment, an increase in hematocrit is indicative of efficacious CVAC treatment. Conversely, a lack of change in the user's hematocrit (or with any of the physiological markers described herein) does not necessarily indicate that the CVAC treatments are not achieving positive results. Similarly, when blood-gas analysis is the physiological marker used to assess CVAC efficacy, modulation of the dissolved gasses in the blood during or following one or more CVAC sessions is indicative of efficacious CVAC treatment. Typical gasses monitored include oxygen, carbon dioxide, and nitrogen. However, any gas found within the blood may be monitored for assessment of CVAC efficacy. When blood-perfusion of the tissues is the physiological marker used to assess CVAC efficacy, increases in blood volumes and/or blood exchange within tissues during or following one or more CVAC sessions are indicative of the efficacious CVAC treatment. Angiogenesis within affected tissues can also be a physiological marker used to assess CVAC efficacy. Modulation of vessel development within the affected tissues during or following one or more CVAC sessions is indicative of efficacious CVAC treatments. Additionally, initiation or modulation of VEGF expression within affected tissues during or following one or more CVAC sessions is also indicative of efficacious CVAC treatment. Modulation of erythropoietin production following one or more CVAC sessions is also a physiological marker used to assess the efficacy of CVAC treatments. In one embodiment of the present invention, increases in the expression of erythropoietin indicate efficacious CVAC treatments. Extent of tissue necropsy is a further physiological marker used to assess CVAC efficacy. Modulation of tissue necropsy, including repair and/or efficient removal of affected tissue by known bodily repair systems, pathways, and cascades as well as prevention of initial or continued necrosis, during or following one or more CVAC sessions is indicative of CVAC session efficacy. Still further physiological markers for assessing efficacy of CVAC sessions include modulation of cognitive and/or motor skills during or following one or more CVAC sessions. In one embodiment, unproved or increased motor skills are indicative of efficacious CVAC treatment. Similarly, in yet another embodiment improved cognitive skills are indicative of efficacious CVAC treatment. Assessment of CVAC efficacy in treating ischemic damage or ischemic events may include all aforementioned techniques and criteria. In addition, efficacy of CVAC session for the treatment, prevention, and/or amelioration of ischemic damage or ischemic events may be assessed by monitoring swelling or fluid collection in body tissues. In one embodiment, the reduction of swelling in the legs and ankles following the administration of one or more CVAC sessions is indicative of efficacious treatment. Additional criteria for assessing the treatment and prevention of ischemic damage or ischemic events may be known by those of skill in the art and can be employed to assess the beneficial effects of CVAC programs.

Alzheimer's Disease

Efficacy of CVAC treatments for Alzheimer's disease can be evaluated with a variety of imaging and assessment techniques known in the art. Examples include methods such as magnetic resonance imaging (MRI) of the affected region, invasive imaging through catheterization, or alternative non-invasive imaging methods. Additional assessment criteria known in the art include: hematocrit measurement, blood-gas analysis, extent of blood-perfusion of tissues, angiogenesis within tissues, erythropoietin production, extent of plaque formation in the affected tissues, and assessment of additional indicators such as speech and cognitive ability, memory and recognition, as well as physical coordination and movement.

Blood Production

CVAC sessions for the production of blood, including but not limited to increased red blood cell counts, increased total blood volume, and increased oxygenation of the blood, are administered preferably for at least 10 minutes, and more preferably at least 20 minutes, with variable frequency. Frequency of sessions or series of sessions may include, but is not limited to, daily, monthly, or when medically indicated or prescribed. The frequency and duration of the sessions can be altered to suit the medical condition to be treated, and CVAC sessions may be administered as single sessions, or as a series of sessions, preferably with a Set-Up Session as described above. Similarly, the targets in the sessions can also be altered or adjusted to suit the individual and medical condition to be treated. The permutations of targets can be customized to the individual, and may again be identified with the help of any person skilled in the art, such as a treating physician. Furthermore, the variations may be administered in regular intervals and sequence, as described, or in random intervals and sequence. The variations in number, frequency, and duration of targets and sessions can be applied to all methods of treatment with CVAC described herein.

Although not limited to a particular mechanism of action, it is believed that the ability of CVAC therapy to provide increased blood flow, increased red blood cell counts, angiogenic and protective cellular responses, EPO production, VEGF production, and HIF production can aid in recovery and repair of damaged tissues as well as modulate production of red blood cells. Additionally, CVAC sessions are believed to act like a vaso-pneumatic pump on the user's body, thus stimulating flow of fluids in the body, including but not limited to blood and lymphatic fluids. The negative and positive pressures imposed by the CVAC session affect the fluid flow or movement within a body, thus improving the delivery of beneficial nutrients, immune factors, blood, and oxygen while also improving the removal of harmful toxins, fluids, and damaged cells or tissues. The combination of the beneficial effects of CVAC sessions results in enhanced production of red blood cells in a user as well as reducing the time required between subsequent blood donations. Improvement may be evidenced by increased oxygenation of the blood and increased hematocrit, allowing for improved frequency of donation as well as improved quality of donated blood.

Specific examples of a CVAC session are shown graphically in FIGS. 1A and 1B. In both figures, the parameters of the program are shown as a line graph with axes that correspond to time (x-axis) and pressure change (y-axis).

A method for production of red blood cells by administration of various environmental pressure levels for hypoxic conditioning is disclosed herein. Previously described PVU and CVAC methodology is used to implement the method for producing red blood cells and alternative PVUs can be used with the disclosed methodologies. Administration of at least one CVAC session or series of sessions aids in the production of blood.

Recent research indicates that altering the cerebral outflow impedance and thus optimizing the compliance (e.g., cradling the brain to prevent excess movement) of the intracranial fluid space may be associated with the likelihood and/or severity of a concussion. Although the skull, blood, and brain are almost incompressible, the vasculature tree of the cerebrum is reactive and compressible.

Hypercarbia can increase the cerebral blood flow and blood volume by up to 40%. What may make the human brain vulnerable to a blast wave is the propensity to oscillate in the skull following a rapid acceleration or deceleration, commonly referred to as Slosh dynamics. The oscillation in the skull is permitted because the cerebrospinal fluid (CSF) is free-flowing and the brain effectively “floats” somewhat unrestrained in the CSF.

During rapid acceleration/deceleration of the skull, such as that which exists during an impact, there is likely a concomitant deformation of brain matter, which is the proposed mechanism of concussion and brain injury. Rapid deformation of the brain can lead to increased shear when tissues of differing mass decelerate at different rates. If one reduces the compliance of the cranial space upon impact, differing tissue densities will likely accelerate or decelerate at the same rate, similar to having an airbag deploy or “bubble wrap” inflate and thus prevent damage to structures within a container (the brain in this example).

In concussion, rapid deformation of brain tissue is thought to cause “diffuse mechanically induced depolarization of cortical neurons.” One hypothesized mechanism of this relationship between volume of intracranial fluid and concussion risk is that a decrease in intracranial fluid results in a relative increase in the amount of space through which the brain travels in the cranium upon impact, resulting in enhanced acceleration/deceleration of both linear and rotational injury inputs to the brain.

Because of the physiologies that occur during acclimatization, including a decline in intracranial compliance (a tighter fit), it was hypothesized that increased altitude (i.e., elevation above sea level) acclimatization likely affects Slosh of the brain during competitive sports. One study conducted an epidemiological field investigation to examine the relationship between altitude and concussion rate in high school sports using data from a large national sample of US high schools. Because of the mild increase in hypoxic vasogenic edema leading to a decrease in compliance (a tight fit) at higher altitudes, it was hypothesized that raised altitude would be related to reduced concussion rates in high school athletes.

Cerebral blood flow rises in response to hypoxemia, including hypoxic changes associated with increased elevations. Restricted venous drainage in the face of this increased cerebral blood flow would result in venous engorgement and a subsequent rise in ICP when the limits of cerebral compliance are reached.

In the chronic phase of adaptation at altitude, erythropoietin stimulates increased blood synthesis with further polycythemia. Initially, hemoglobin and hematocrit rise and blood volume declines, but as acclimatization occurs, an increase in erythrocyte volume is manifested. In long-term acclimatization, increased arterial oxygen content is sustained by expansion of erythrocyte volume. Vasogenic edema in the brain leads to increased extravascular water. These 2 adaptations would also lead to tighter packaging of the brain (less compliance ¼ tight fit) with increased blood cell content surrounding the brain. It has been noted that cerebral volume must rise by only 3 to 4 mL before pressure starts to rise and take up the compliance with brain tissues.

The Slosh energy management model predicts reduced concussion rates at higher altitudes due to modified physiologies resulting from acclimatization to altitude. Increased DPG, red cell mass, intracranial volume, and decreased compliance (a “tight fit”), or a combination thereof, can mitigate Slosh, and the study's results confirm this conclusion. [See Smith et al, Altitude Modulates Concussion Incidence: Implications for Optimizing Brain Compliance to Prevent Brain Injury in Athletes, Orthopaedic Journal of Sports Medicine November 2013 vol. 1 no. 6, DOI: 10.1177/2325967113511588]. See also US2011/0065637, Method To Reduce SLOSH Energy Absorption And Its Damaging Effects Through The Reduction Of Inelastic Collisions In An Organism.

As described in International Patent Appl. No. PCT/US2008/054923 to Linton et al., filed Feb. 25, 2008, and entitled, “Combination Pressure Therapy for Treatment of Serum Lipid Levels, Steroid Levels, and Steroidogenesis,” the entire disclosure of which is incorporated herein by reference, while oxygen deprivation of the body or specific tissues can cause tissue damage, and even death, controlled deprivation of oxygen to the body and/or specific tissues has been shown to be beneficial when imposed for specific periods of time under particular conditions. In practice, most current hypoxic conditioning protocols utilize static pressures for blocks of time ranging from 30 minutes to an hour or more to achieve the desired and reported responses. Hypoxic conditioning may be provided by decreased oxygen levels in the atmosphere or by a reduction in atmospheric pressure (hypobaric conditions), thus reducing the availability of oxygen for efficient respiration. Both methods can provide beneficial results including protection of tissues from damage due to injury and ischemia.

As described in International Patent Appl. No. PCT/US2008/054923 to Linton et al., filed Feb. 25, 2008, and entitled, “Combination Pressure Therapy for Treatment of Serum Lipid Levels, Steroid Levels, and Steroidogenesis,” the entire disclosure of which is incorporated herein by reference, while oxygen deprivation of the body or specific tissues can cause tissue damage, and even death, controlled deprivation of oxygen to the body and/or specific tissues has been shown to be beneficial when imposed for specific periods of time under particular conditions. In practice, most current hypoxic conditioning protocols utilize static pressures for blocks of time ranging from 30 minutes to an hour or more to achieve the desired and reported responses. Hypoxic conditioning may be provided by decreased oxygen levels in the atmosphere or by a reduction in atmospheric pressure (hypobaric conditions), thus reducing the availability of oxygen for efficient respiration. Both methods can provide beneficial results including protection of tissues from damage due to injury and ischemia.

Static hypoxic therapy for extended durations of time has been shown to significantly reduce total cholesterol, LDL, very low-density lipoprotein (VLDL), as well as increase HDL. Thus, the overall serum lipid profile was also significantly reduced. [Tin'Kov, A. N. and Aksenov, V. A., Effects of Intermittent Hypobaric Hypoxia on Blood Lipid Concentrations in Male Coronary Heart Disease Patients, High Alt. Med. & Biol., 3(3): 277-282 (2002)]. Type 2 Diabetes has been regarded as a relatively distinct disease entity, but recent understanding has revealed that Type 2 Diabetes (and its associated hyperglycemia or dysglycemia) is often a manifestation of a much broader underlying disorder, which includes metabolic syndrome. This syndrome may also be referred to as Syndrome X, and is a cluster of cardiovascular disease risk factors that, in addition to glucose intolerance, includes hyperinsulinaemia, dyslipidaemia, hypertension, visceral obesity, hypercoagulability, and micro albuminuria. Provided herein are methods of treating metabolic syndrome and/or insulin resistance. In one embodiment, metabolic syndrome is treated by modulation of testosterone levels via application of at least one CVAC session.

Additionally, application of physical energy or force to the body through relatively low levels vibrational therapy has been linked to increases in steroidogenesis, [Bosco, C. et al., Hormonal responses to whole-body vibration in men, Eur. I. Appl. Physiol., 81: 449-454 (2000)], and application of physical force to the epidermal layers of the skin through endermologie has also been shown to modulate estradiol (an estrogen) levels in women. [Benelli, L., et al., Enderrnologie: humoral repercussions and estrogen interaction, Aesthetic Plast. Surg. 23(5): 312-15 (1999)].

There is a high prevalence of low testosterone levels in HIV-infected individuals, and 20-25% of HIV-infected men who receive highly active antiretroviral therapy (HAART) also suffer from reduce testosterone levels. Furthermore, low testosterone levels are associated with weight loss, progression to AIDS, wasting, depression and loss of muscle mass. [Bahsin et al., Testosterone Therapy in Adult Men with Androgen Deficiency Syndromes: An Endocrine Society Clinical Practice Guideline, I. elin. Endocrin. & Metab., 91(6): 1995-2010 (2006); Arver et al., Serum Dihydrotestosterone and testosterone concentrations in Human Immunodeficiency Virus-infected men with and without weight loss, J. Andrology, 20(5):611-618 (1999)]. Testosterone therapy in HIV-infected individuals is known to improve weight gain, improve muscle strength, and provide gains in lean-body mass. Provided herein are methods for modulating steroidogenesis in HIV-infected individuals. In one non-limiting example, administration of at least one CVAC session to an HIV-infected individual increases testosterone levels in the HIV-infected individual.

Abnormalities in serum lipid levels and the process of steroidogenesis (including the modulation of steroid levels) are commonly treated with pharmaceuticals. Examples of such pharmaceuticals include, but are not limited to, Lipitor®, Zocor®, Vytorin®, and other statins as well as supplemental testosterone, estrogens, and other hormones. There is a need for alternative therapies for modulation of serum lipid levels, the modulation of steroidogenesis, and the modulation of steroid levels.

By use of the present invention, CVAC sessions can modulate steroid levels and/or steroidogenesis in a subject. Examples of steroids modulated include, but are not limited to, testosterone and estrogen, The combination of the beneficial effects of CVAC sessions results in treatment and modulation of serum lipids and/or the modulation of steroidogenesis and steroid levels, including all the aforementioned aspects and embodiments.

Methods of using air pressure therapy for the treatment and prevention of diseases and conditions, and, more specifically, using whole body hypobaric conditioning and/or whole body vaso-pneumatic compression for the treatment of loss of sensation and/or chronic pain, are also disclosed herein. As used herein, “chronic pain” refers to pain that occurs over an extended duration (e.g., at least about three or six months) and can include, but is not limited to, headaches, back or neck pain, arthritis pain, carpal tunnel syndrome, fibromyalgia/fibrosis, myofascial pain, neuropathy and neuralgia pain, phantom limb pain. Chronic pain can also include pain associated with an illness or condition such as, for example, adiposis dolorosa, diabetes, osteoporosis, lupus, rheumatoid arthritis, scoliosis, endometriosis, and scleroderma. Such methods can include the use of whole body cyclic pneumatic hypobaric compression for the treatment of chronic pain associated with a specific condition, including, for example, diabetic neuropathy, such as diabetic peripheral neuropathy, fibromyalgia, and/or adiposis dolorosa.

Targets in the sessions can also be altered or adjusted to suit the individual and medical condition to be treated. If at any time the user or attendant determines that the session is not being tolerated well, an abort may be initiated and the user brought down safely and exited. The permutations of targets can be customized to the individual, and may again be identified with the help of any person skilled in the art, such as a treating physician. Furthermore, the variations may be administered in regular intervals and sequence, as described, or in random intervals and sequence. The variations in number, frequency, and duration of targets and sessions can be applied to all methods of treatment with CVAC described herein. Treat or treatment, as used herein refers to the treatment of a disease or disorder related to abnormal levels of lipids. This includes, but is not limited to, inhibiting the disease or disorder, arresting the development of the disease or disorder, relieving the disease or disorder, or stopping the symptoms of the disease or disorder. Thus, as used herein, the term “treatment” is used synonymously with the terms “alleviation,” “amelioration,” “prophylaxis,” or “prevention.” Treatment can refer to a reduction in lipid levels compared to no treatment (e.g. about 1% less, about 2% less, about 3% less, about 4% less, about 5% less, about 10% less, about 20% less, about 50% less, about 100% less, and any range therein). Treat or treatment, as used herein, can also refer to the treatment of a disease or disorder, and more specifically to the treatment of loss of sensation or chronic pain. Treatment can refer to a reduction in perceived chronic pain levels compared to no treatment (e.g. about 1% less, about 2% less, about 3% less, about 4% less, about 5% less, about 10% less, about 20% less, about 50% less, about 100% less, and any range therein).

Cyclic Variations in Altitude Conditioning Program (CVAC) may be used to treat users who wish to modulate their serum lipid levels. CVAC is administered to stimulate the reduction in serum lipid levels in a user as well as stimulate other associated physiological processes affected by CVAC treatment such as fluid movement, vas-pneumatic pressure on the user, and the cellular processes initiated by hypoxic exposure. Treatment is administered through the use of one or more CVAC sessions. Such sessions may be user defined or custom-defined with input from the user's physician. In an embodiment of the present invention, Cyclic Variations in Altitude Conditioning Program (CVAC) is used to treat users who wish to lower their serum lipid levels. In another embodiment of the present invention, CVAC is used to modulate LDL. In another embodiment of the present invention, CVAC is used to modulate cholesterol. In another embodiment of the present invention, CVAC is used to modulate VLDL (very low-density lipoprotein). In yet another embodiment of the present invention, CVAC is used to modulate HDL. In further embodiments, two or more of, in any combination of, cholesterol, VLDL, LDL, and HDL can be modulated by the same application of at least one CVAC session.

In another aspect of the present invention, CVAC sessions are administered for the modulation of steroidogenesis. As described herein, modulation of steroidogenesis includes, but is not limited to, increases and decreases in steroid levels in the user. Steroidogenesis includes, but is not limited to, the production of steroids. Steroid as used herein includes, but is not limited to, all hormones and steroid compounds produced from cholesterol. Examples of groups of such compounds include androgens, estrogens, progestogens, mineralocorticoids, and gluococorticoids. Further examples of hormones include testosterone and estrogens. Still further examples of estrogens include estradiols, estriols, and estrones. Similarly, the treatment of steroidogenesis includes administration for modulation of steroid levels and steroidogenesis. CVAC sessions for the treatment of steroidogenesis are administered preferably for at least 10 minutes, and more preferably at least 20 minutes, with variable frequency. Additional administration periods may include, but are not limited to, about 10 minutes, about 20 minutes, about 30 minutes, about 40 minutes, about 60 minutes, between 10 and 20 minutes, between 20 and 30 minutes, between 30 and 60 minutes, and between 60 and 120 minutes. Frequencies of sessions or series of sessions may include, but are not limited to, daily, monthly, or when medically indicated or prescribed. The frequency and duration of the sessions can be altered to suit the medical condition to be treated, and CVAC sessions may be administered as single sessions, or as a series of sessions, preferably with a Set-Up Session as described herein. For example, the frequency of sessions or series of sessions can be administered 3 times a week for 8 weeks, 4 times a week for 8 weeks, 5 times a week for 8 weeks, or 6 times a week for 8 weeks. Additional frequencies can be easily created for each individual user. Similarly, the targets in the sessions can also be altered or adjusted to suit the individual and medical condition to be treated.

The permutations of targets can be customized to the individual, and may again be identified with the help of any person skilled in the art, such as a treating physician. Furthermore, the variations may be administered in regular intervals and sequence, as described, or in random intervals and sequence. The variations in number, frequency, and duration of targets and sessions can be applied to all methods of treatment with CVAC described herein. As used herein, “modulation” includes increases or decreases in steroidogenesis as well as increases or decreases in serum and/or tissue steroid levels. Modulation can refer to increases in serum or tissue steroid levels compared to no treatment (e.g. about 1% more, about 2% more, about 3% more, about 4% more, about 5% more, about 10% more, about 20% more, about 50% more, about 100% more, and any range therein).

In an embodiment of the present invention, CVAC is administered to increase the levels of testosterone in the user. In a further embodiment, CVAC is administered to modulate levels of steroids in an HIV-infected or HIV-positive individual. In one non-limiting example, at least one CVAC session is administered to and HIV-infected individual to increase the levels of testosterone in the HIV-infected individual. In an additional embodiment, CVAC is administered to a user to increase the levels of estrogen in an HIV-infected user. In an additional embodiment, CVAC is administered to a user to decrease the levels of testosterone or estrogen in the user. In yet another embodiment, CVAC is administered to a user to modulate the levels of glucocorticoids, mineralocorticoids, or androgens. In still further embodiments, CVAC is administered to modulate steroid levels and cholesterol levels in an HIV-infected user. In still further embodiments, CVAC is administered to modulate both steroid levels and serum lipid levels in an HIV-infected user. In further embodiments, at least one CVAC session is administered to increase steroid levels in an HIV-infected subject for the treatment of weight loss, wasting syndrome, or loss of muscle mass. Treatment is administered through the use of one or more CVAC sessions. Such sessions may be user defined or custom-defined with input from the user's physician.

In yet another embodiment, at least one CVAC session is administered to a user to modulate steroid levels in a subject for the treatment, prevention or amelioration of metabolic syndrome. In additional embodiment, at least one CVAC session is administered to modulate steroid levels in an individual for the treatment prevention or amelioration of type-2 diabetes. In yet another embodiment, at least one CVAC session is administered to modulate steroid levels in an individual for the treatment, prevention or amelioration of insulin resistance. In a further embodiment, at least one CVAC session is administered to increase steroid levels in a subject for the treatment of metabolic syndrome. In another embodiment, at least one CVAC session is administered to increase steroid levels in a subject for the treatment of type-2 diabetes. In yet another embodiment, at least one CVAC session is administered to increase steroid levels in a subject for the treatment of insulin resistance. In one non-limiting example, at least one CVAC session is administered to increase testosterone in a subject for the treatment of metabolic syndrome. In another non-limiting example, at least one CVAC session is administered to increase testosterone in a subject for the treatment of type-2 diabetes. In additional embodiments, CVAC sessions are administered to increase steroid levels for the prevention of metabolic syndrome or insulin resistance.

Assessment of CVAC efficacy in the aforementioned aspects and embodiments can be investigated through various physiological parameters. Changes in serum lipid levels can be assessed by evaluation of cholesterol, VLDL, LDL, and HDL levels in a user. By example only, when levels of LDL are the physiological parameter examined, decreases in the levels of LDL in a user's blood or serum are indicative of efficacious CVAC treatments. Similarly, when the physiological parameter is cholesterol, reductions in cholesterol levels are indicative of efficacious CVAC treatment. Serum steroid and hormone levels can be assayed via RIA, ELISA, immunometric assays, equilibrium dialysis, or liquid chromatography tandem mass spectrometry. Additional steroid and hormone assays are known in the art and contemplated herein. In one example, serum total testosterone is determined by RIA, with free testosterone determined by equilibrium dialysis. Additionally, weight gain, increases in lean-body mass, and/or increases in muscle strength indicate efficacy of CVAC for increasing steroid levels in an HIV-infected subject.

Further methods of assessing CVAC efficacy for changes in serum lipid levels include non-invasive imaging techniques such as MRI as well as invasive imaging techniques such as catheterization and endoscopy. Additional imaging techniques may be well known in the art and easily applied to the present invention.

When treating or modulating steroidogenesis and/or steroid levels, a user's steroid or hormone levels may be assessed for determination of CVAC efficacy. For but one example only, when testosterone is the physiological parameter assessed, increases in testosterone levels can be indicative of efficacious CVAC treatment. Similarly, increases in estrogen levels can be indicative of efficacious CVAC treatment. In further embodiments, modulation of a user's androgen levels, progestogen levels, mineralocorticoid levels, or glucocorticoid levels are indicative of efficacious CVAC treatment. In still further embodiments, decreases in a user's androgen levels, progestogen levels, mineralocorticoid levels, or glucocorticoid levels are indicative of efficacious CVAC treatment.

Additionally, increases in a subjects weight, muscle mass, or lean-body mass are indicative of efficacious CVAC treatment for increasing steroid levels in an HIV-infected subject. Similarly, increases in muscle strength can also be are indicative of efficacious CVAC treatment for increasing steroid levels in an HIV-infected subject. Established methods of monitoring and assessing weight gain, muscle mass, lean-body mass, and muscle strength are known in the art and contemplated herein.

Efficacy of CVAC treatments for modulation of steroid levels for the treatment of metabolic syndrome, type-2 diabetes, or insulin resistance can be evaluated by assessment of insulin regulation, glucose tolerance, and glucose transport. Assays for such criteria are well known in the art and can be evaluated with a variety of imaging and assessment techniques. By example only, increase of insulin levels is indicative of efficacious CVAC treatments for modulation of steroid levels to treat metabolic syndrome type-2 diabetes, or insulin resistance. Similarly, a decrease of glucose levels is indicative of efficacious CVAC treatment for the modulation of steroid levels to treat metabolic syndrome, type-2 diabetes, or insulin resistance, and modulation of glucose transport is indicative of CVAC efficacy for the modulation of steroid levels to treat metabolic syndrome, type-2 diabetes, or insulin resistance.

A lack of change in the user's insulin (or with any of the physiological markers described herein) does not necessarily indicate that the CVAC treatments are not achieving positive results. Efficacy of CVAC sessions for the modulation of steroid levels to treat metabolic syndrome, type-2 diabetes, or insulin resistance can also be determined by assessment of testosterone levels in a user, as described above.

Additional criteria for assessing the efficacy of the aforementioned aspects and embodiments may be known by those of skill in the art and can be employed to assess the beneficial effects of CVAC programs

Methods for treating serum lipid levels and treating steroidogenesis by administration of various environmental pressure levels for hypoxic conditioning are disclosed herein. Previously described PVU and CVAC methodology is used to implement the methods for treatment of the aforementioned conditions, and alternative PVUs can be used with the disclosed methodologies.

### EXAMPLES

### Example 1

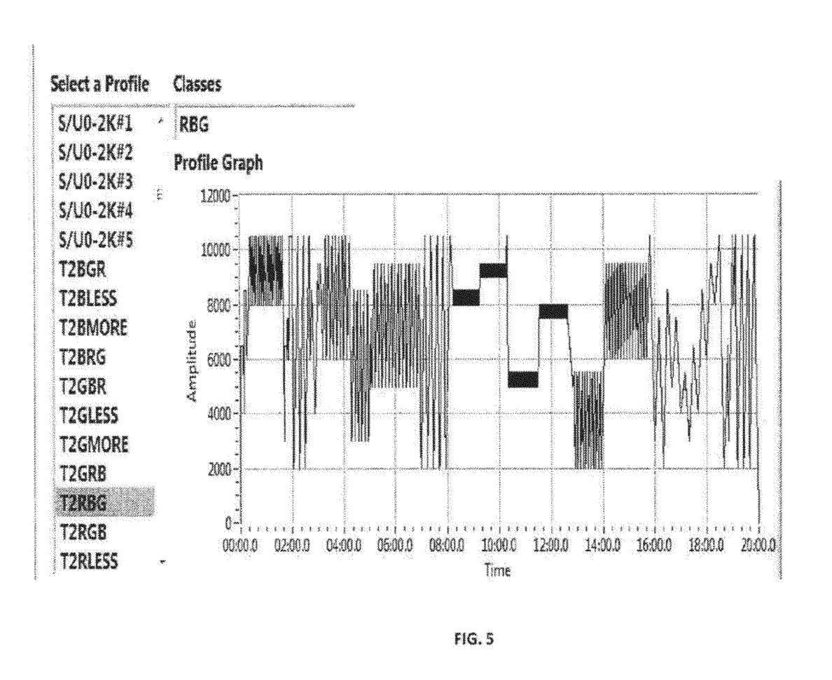

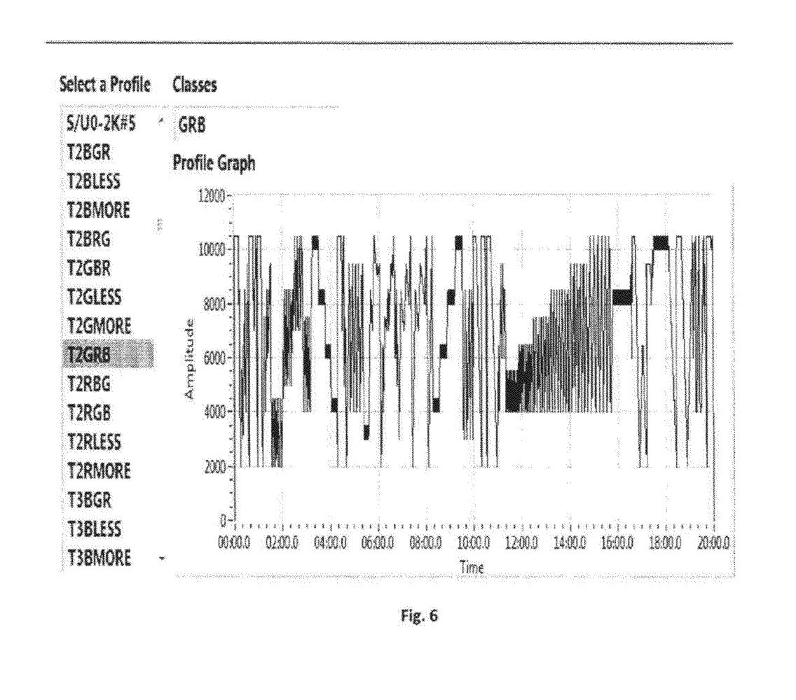

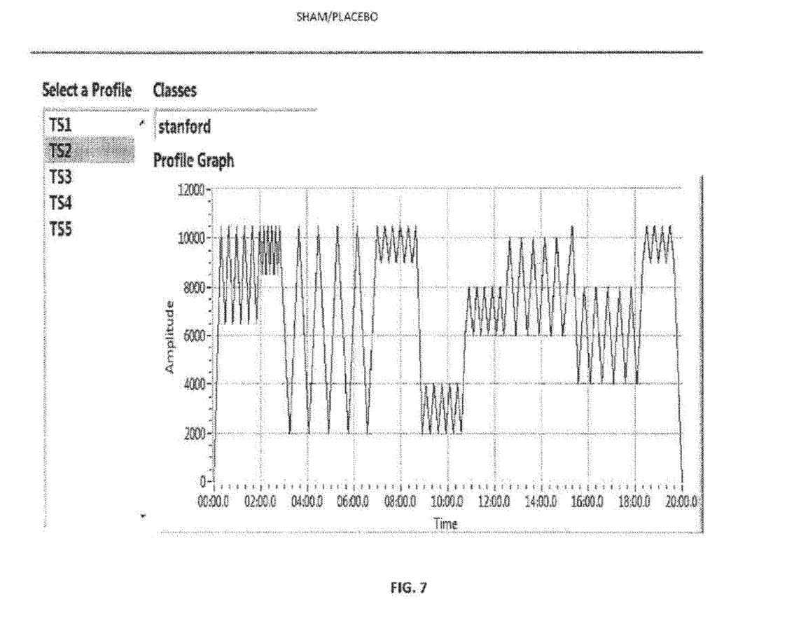

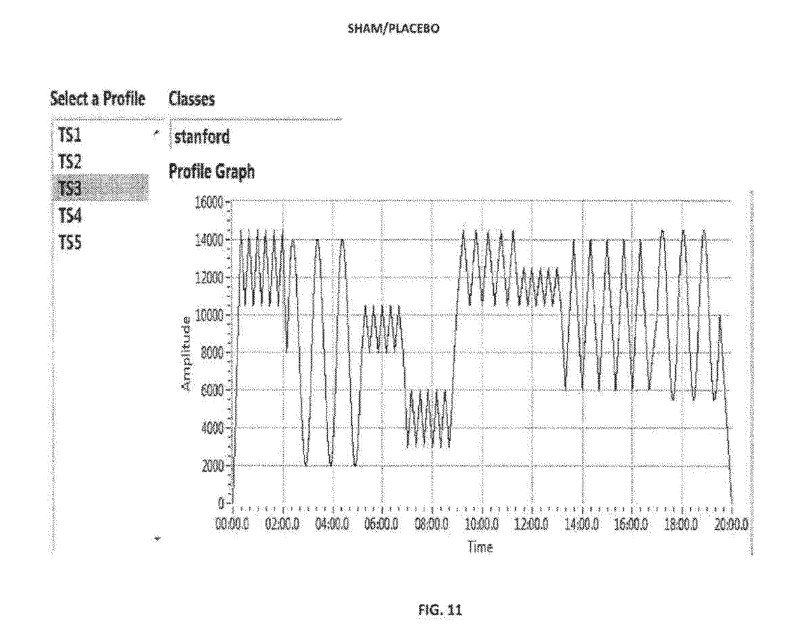

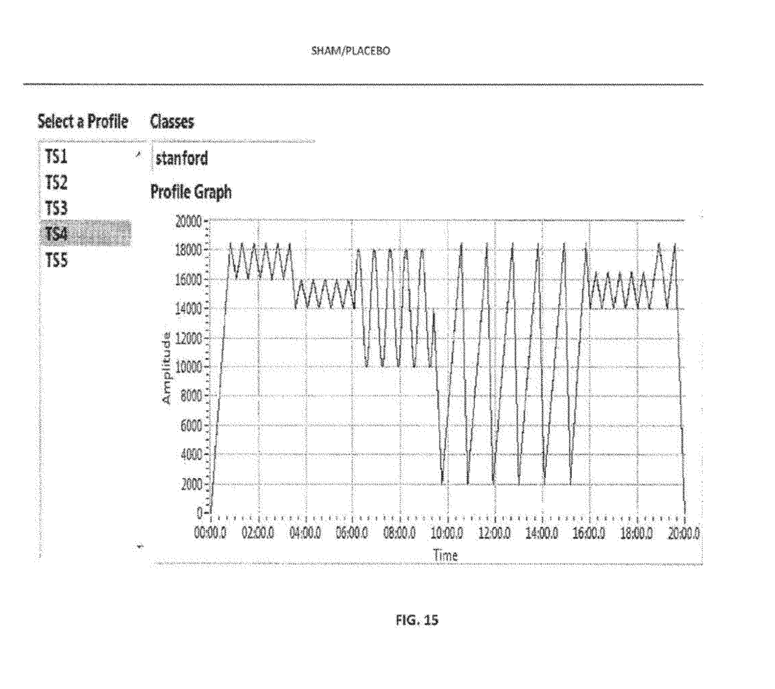

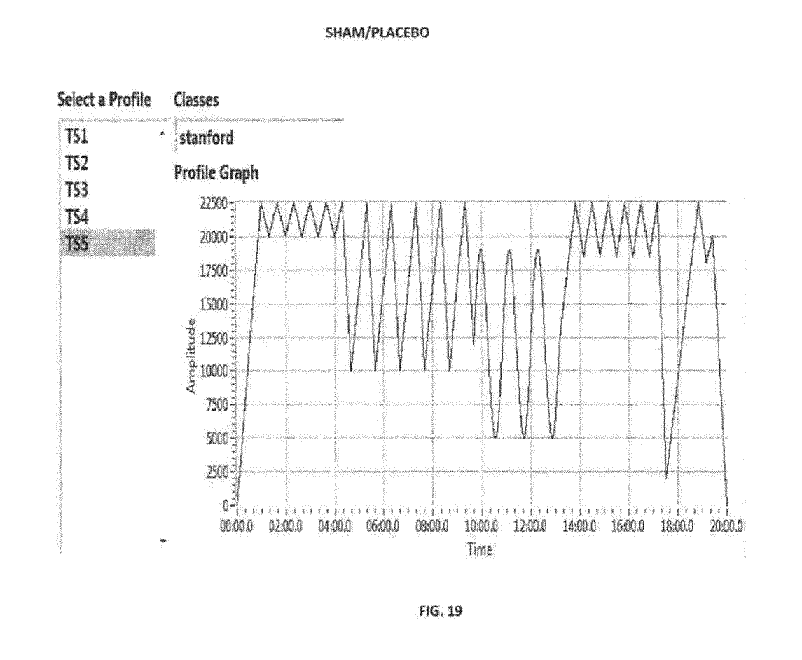

To assess the efficacy of CVAC sessions, 13 individuals, all between the ages of 20 and 40 years old, were administered CVAC sessions and changes in their erythropoietin (EPO) levels were measured. Frequency of CVAC administration was 3 CVAC sessions per day, 5 days per week, for seven weeks. All subjects were administered three different profiles, entitled BRO, RBO, and ORB. Each CVAC session profile cycled through a rotation of the pressures and parameters associated with that given profile. After completing three 20-minute CVAC sessions including a given profile, each subject then switched to a second CVAC session profile. The subjects then experienced three CVAC sessions of this second profile before switching to the third CVAC session profile. After completion of three CVAC sessions based on the third profile, the subject then returned to the first profile, with each profile be repeated in triad form. All CVAC sessions, regardless of the profile used, had a pressure ceiling corresponding to a specific tier. Subjects then progressed through five tiers, and each tiered level included a maximum pressure ceiling that corresponded to an altitude of 4000 feet higher than the previous tier. A subject was not allowed to switch to the next higher tier until the subject had experienced fifteen CVAC sessions at the lower tier. Sham sessions (or control sessions) correspond to the cycling of the five tier levels but do not contain any meaningful pressure changes (e.g. pressure changes equivalent to altitude of 2000 feet with very few changes in duration), thus the subjects experience the CVAC session for the equivalent 20 minute session, but without the pressure changes and durations. In this study, profiles BRG (FIGS. 4, 8, 12, and 16), RBG (FIGS. 5, 9, 13, and 17), GRB (FIGS. 6, 10, 14, and 18) (tiers 2-5 respectively) were administered in sequential order for tiers 2-5 as described above. Sham sessions corresponding to tiers 2, 3, 4, and five (FIGS. 7, 11, 15, and 19) were administered where indicated and the graphical representations corresponding to pressures are not indicative of the pressure changes in the CVAC unit. The simulated graphical output was for control purposes to keep the subjects blinded to the sham sessions.

Increases in EPO were measured prior to administration of CVAC and three hours post-administration of CVAC, and EPO concentration is expressed as mIU/ml. Thus changes in EPO can be represented by the formula: deltaEPO=Post-CVAC EPO mIU/ml-pre-CVAC EPO mIU/ml. The study found that EPO levels changed over the study period in the population. Specifically, mean changes in EPO concentration increased from 0.2 mIU/ml following the first 2 weeks of CVAC administration to 2.0 mIU/ml following 8 weeks of the CVAC administration. The changes in EPO levels found in the study population indicate that the administration of CVAC sessions can positively modulate EPO production, hence providing an alternative and efficacious method to exogenous EPO administration.

### Example 2

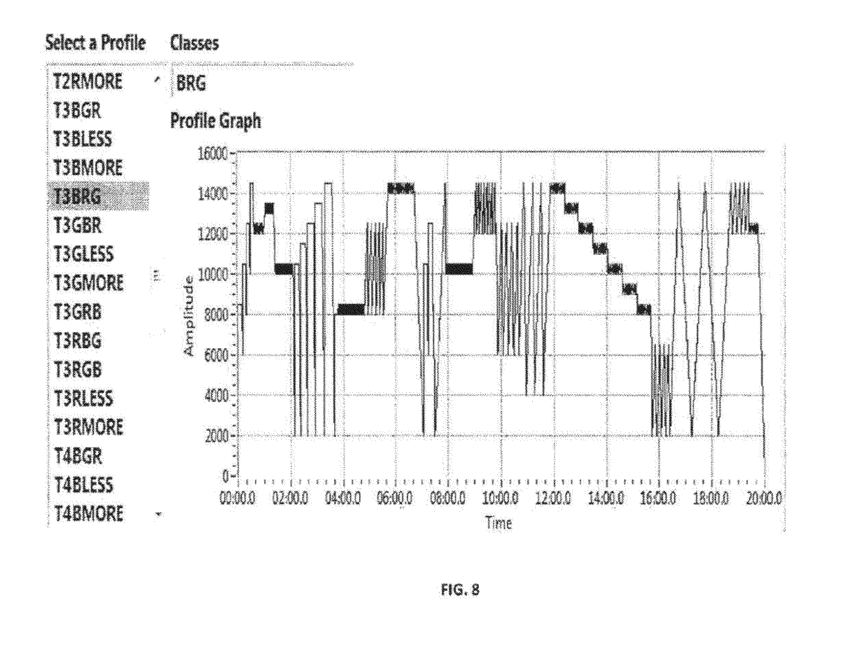

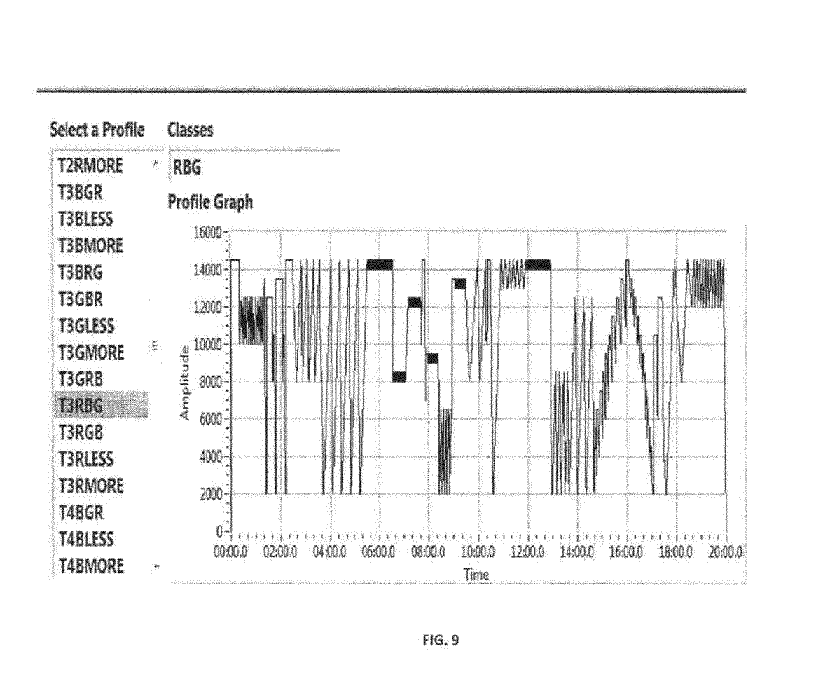

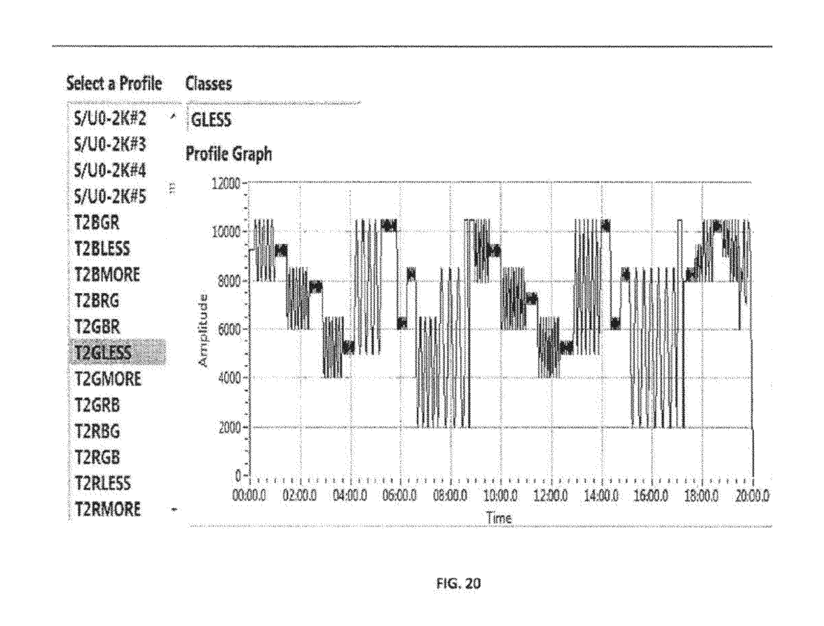

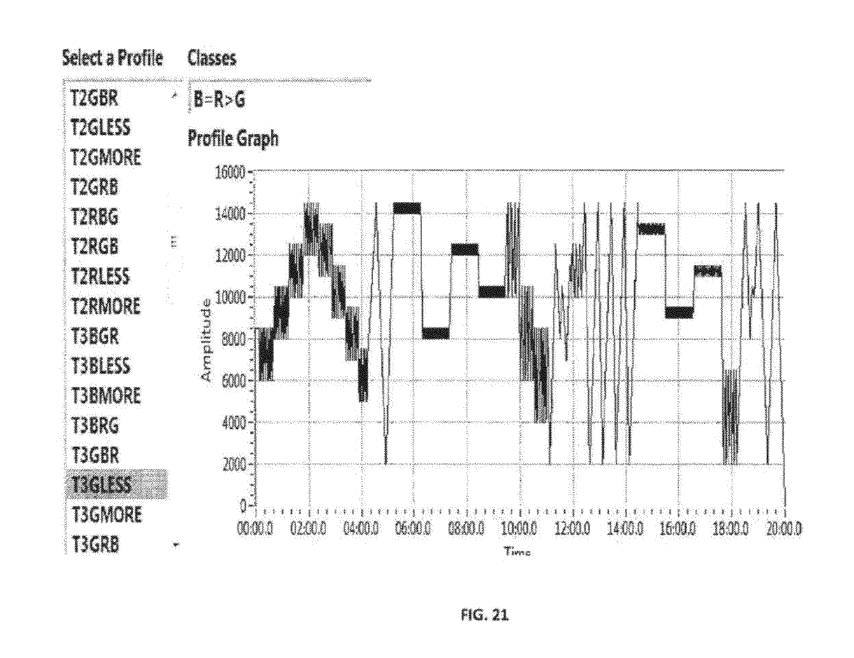

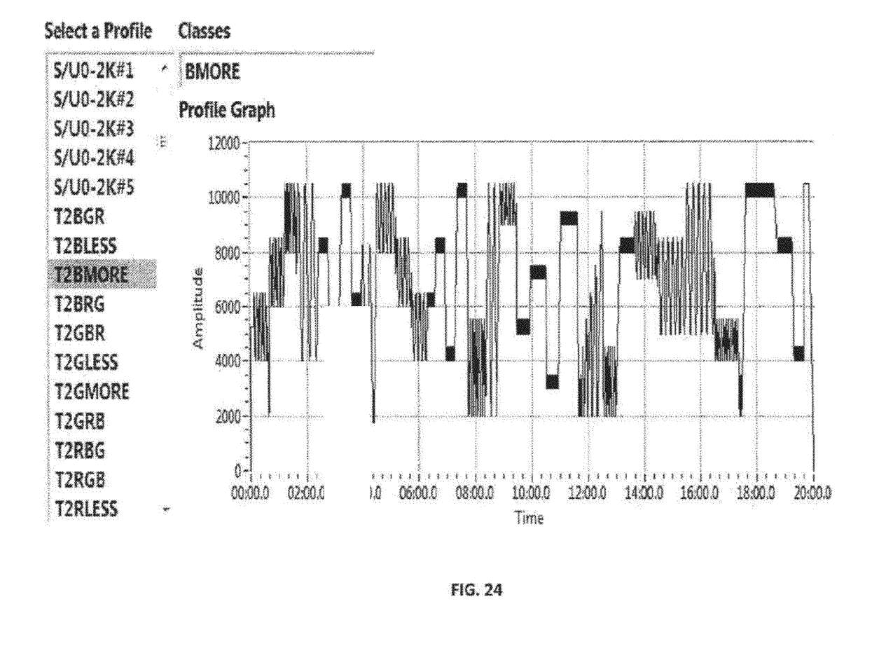

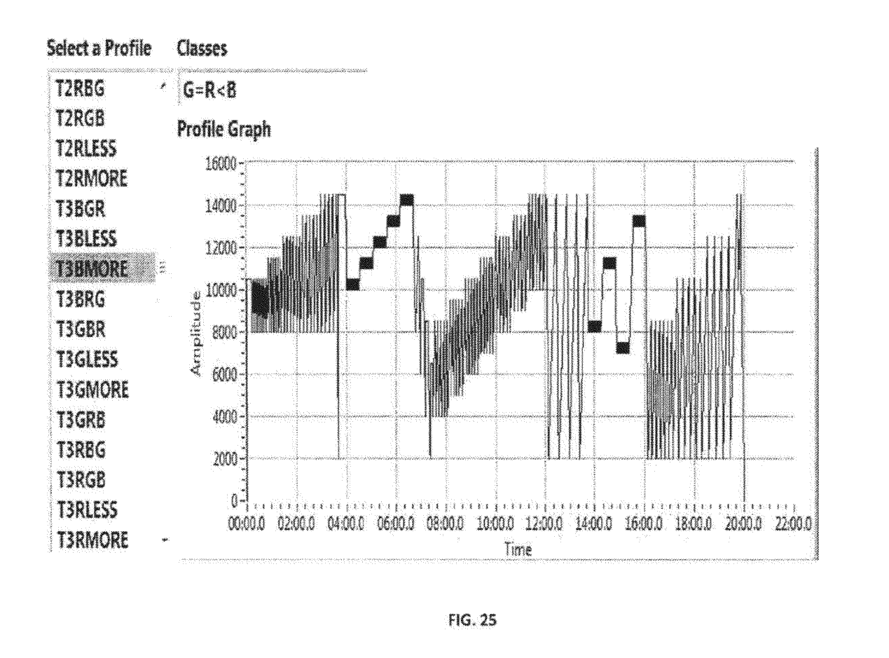

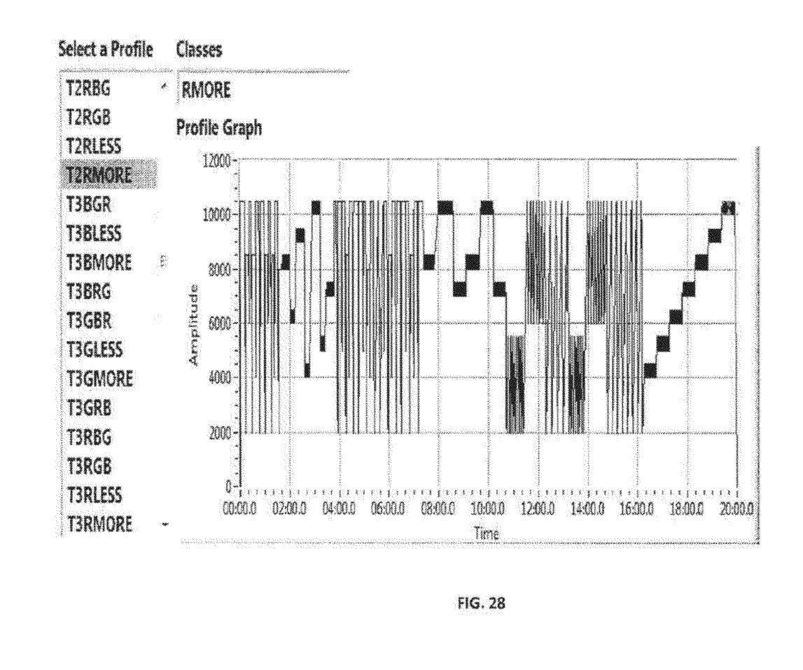

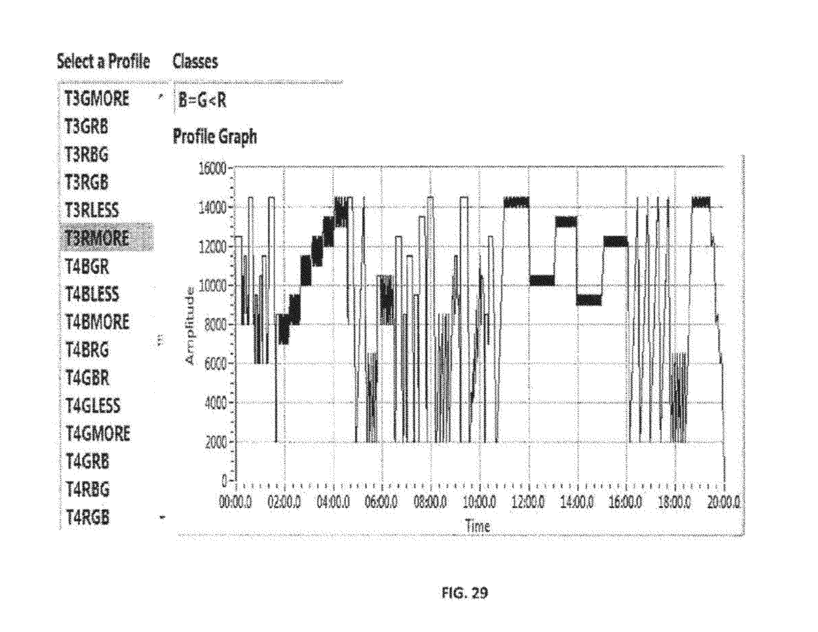

Two diabetic subjects (Type-1 and Type-2) were administered 20 minute CVAC sessions, three times a week over a 9 week period. Subject #1 was administered a rotation classified as GLESS, which included profiles, for tiers 2 and 3 respectively, GLESS (FIGS. 20, 21), BMORE (FIGS. 24, 25), RMORE (FIGS. 28, 29), RBG (FIGS. 5, 9), and BRG (FIGS. 4, 8). Subject #2 was administered a rotation classified as BRG, which comprised profiles BRG (FIGS. 4, 8), RBG (FIGS. 5, 9), GLESS (FIGS. 20, 21), RMORE (FIGS. 28, 29), and BMORE (FIGS. 24, 25). Triglycerides (TGC), Cholesterol levels (HDL and LDL), and Hemoglobin A1c levels were assessed during the study period. Subject #1 underwent additional CVAC sessions and was additionally assessed at a 14-week time-point. Study time periods and results are shown in Table 1.

TABLE 1

| | Baseline | | 9 Weeks | | 14 Weeks | |
|---|---|---|---|---|---|---|
| Physiological | Subject | Subject | Subject | Subject | Subject | Subject |
| Marker | #1 | #2 | #1 | #2 | #1 | #2 |
| Triglycerides | 102 | 81 | 118 | 85 | 101 | n/d* |
| (TGC) | | | | | | |
| HDL | 49 | 72 | 49 | 76 | 49 | n/d* |
| LDL | 106 | 111 | 67 | 99 | 84 | n/d* |
| HbA1c | 6.7 | 8.4 | 6.8 | 7.6 | 7.1 | n/d* |
| (LDL + TGC)/ | 4.2 | 2.7 | 3.8 | 2.4 | 2.1 | n/d* |
| HDL | | | | | | |

Subject #1: Type-2 diabetic, female
Subject #2: Type-1 diabetic, male
*n/d = not determined

The results from the two different subjects show a decrease in their (LDL+TGC)IHDL ratios, indicating improvement in HDL as well as reductions in LDL and/or TGC. Thus in this study, the administration of CVAC sessions resulted in a greater than 10% reduction in the (LDL+TGC)/HDL ratio in subject #2, and a 50% reduction in subject #1. Further, CVAC successfully reduced the LDL and TGC levels of both diabetic individuals, and raised the HDL levels in the diabetic individuals. Thus, in some embodiments, the application of at least one CVAC session may result in at least a 5% reduction in the (LDL+TGC)IHDL ratio, at least a 5-10% reduction in the (LDL+TGC)IHDL ratio, or greater than a 10% reduction in the (LDL+TGC)/HDL ration.

### Example 3

A 36 year old male was administered CVAC sessions for 40 minutes (two twenty-minute CVAC sessions administered in immediate succession), 4 times a week for 12 weeks. In this study, the CVAC session rotation was classified as REG which included five profiles, for tiers 2-5, REG (FIGS. 5,9,13, and 17), BRG (FIGS. 4, 8, 12, and 16), RMORE (FIGS. 28, 29, 30, and 31), GLESS (FIGS. 20, 21, 22, and 23), and REG again. Testosterone (T) levels, total testosterone levels (TT), LDL levels (LDL), Total Cholesterol (C), and Insulin levels (1) were assessed. Results of physical markers prior to CVAC treatment and after CVAC treatment are shown in Table 2.

TABLE 2

| | 3 months prior to | 3 months after beginning |
|---|---|---|
| | CVAC treatment | CVAC treatment |
| Physiological Marker | Subject #1 | Subject #1 |
| Free Testosterone (T) | 80 | 177 |
| Total Testosterone (TT) | 298 | 706 |
| Total Cholesterol (C) | 275 | 258 |
| Serum LDL | 208 | 191 |
| Serum Insulin (I) | 5.0 | 2.0 |

The results of the study demonstrate that CVAC administration increased T levels while also decreasing LDL, C, and I. Specifically, LDL was reduced by 9%, T was increased by 121%, TT was increased by 58%, and I was reduced by 60%. Thus, in some embodiments, the application of at least one CVAC session may result in at least a 10% increase in T, at least a 20% increase in T, at least a 30% increase in T, at least a 40% increase in T, at least a 50% increase in T, at least a 75% increase in T, at least a 100% increase in T, or greater than a 100% increase in T. Similarly, the application of at least one CVAC session may result in at least a 1% reduction in LDL, at least a 2% reduction in LDL, at least a 3% reduction in LDL, at least a 4% reduction in LOL, at least a 5% reduction in LOL, at least a 10% reduction in LOL, or greater than a 10% reduction in LDL. The application of at least one CVAC session may further result in at least a 1% reduction in serum insulin, at least a 5% reduction in serum insulin, at least a 10% reduction in serum insulin, at least a 20% reduction in serum insulin, at least a 30% reduction in serum insulin, at least a 60% reduction in serum insulin, or greater than a 60% reduction in serum insulin.

### Example 4

Effect of CVAC exposure of 40 minutes twice a week on endogenous testosterone. Six subjects (S-I, S-3, S-6, M-9, M-18, and M-23) and a control subject (M-14) are administered two twenty-minute CVAC sessions, administered in immediate succession, twice a week throughout the study period. The CVAC sessions experienced by each subject included a profile of pressure levels and durations for each pressure level. There were three different profiles used in the study, entitled BRG, RBG, and GRB′ Each CVAC session profile cycled through a rotation of the pressures and parameters associated with that given profile. After completing three 20-minute CVAC sessions including a given profile, each subject then switched to a second CVAC session profile. The subjects then experienced three CVAC sessions of this second profile before switching to the third CVAC session profile. After completion of three CVAC sessions based on the third profile, the subject then returned to the first profile, with each profile be repeated in triad form. All CVAC sessions, regardless of the profile used, had a pressure ceiling corresponding to a specific tier. Subjects then progressed through tiers 2-5, and each tiered level included a maximum pressure ceiling that corresponded to an altitude of 4000 feet higher than the previous tier. A subject was not allowed to switch to the next higher tier until the subject had experienced fifteen CVAC sessions at the lower tier. Sham sessions (or control sessions) correspond to the cycling of the five tier levels but do not contain any meaningful pressure changes (e.g. pressure changes equivalent to altitude of 2000 feet with very few changes in duration), thus the subjects experience the CVAC session for the equivalent 20 minute session, but without the pressure changes and durations. In this study, profiles BRG (FIGS. 4, 8, 12, and 16), RBG (FIGS. 5, 9, 13, and 17), GRB (FIGS. 6, 10, 14, and 18) (tiers 2-5 respectively) were administered in sequential order for tiers 2-5 as described above. Sham sessions corresponding to tiers 2, 3, 4, and five (FIGS. 7, 11, 15, and 19) were administered where indicated and the graphical representations corresponding to pressures are not indicative of the pressure changes in the CVAC unit. The simulated graphical output was for control purposes to keep the subjects blinded to the sham sessions.

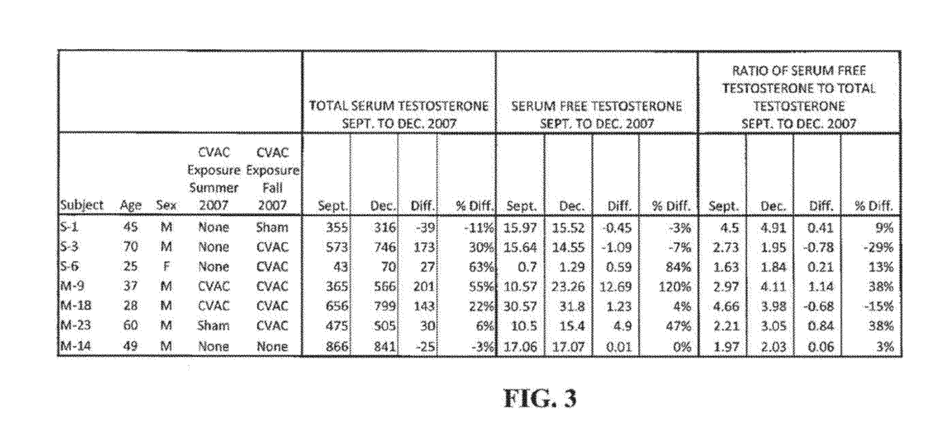

Blood samples were drawn prior to beginning the study period and after the final CVAC session at the end of the study period. Blood samples were analyzed for total testosterone, free testosterone, and the ratio of total testosterone to free testosterone. Results are shown in FIG. 3.

### Example 5

Effect of CVAC exposure of 40 minutes twice a week on serum lipid levels. Six subjects (S-I, S-3, S-6, M-9, M-18, and M-23) and a control subject (M-14) are administered two twenty-minute CVAC sessions, twice a week for throughout the study period. The CVAC sessions experienced by each subject included a profile of pressure levels and durations for each pressure level. There were three different profiles used in the study, entitled BRG, RBG, and GRB. Each CVAC session profile cycled through a rotation of the pressures and parameters associated with that given profile. After completing three 20-minute CVAC sessions including a given profile, each subject then switched to a second CVAC session profile. The subjects then experienced three CVAC sessions of this second profile before switching to the third CVAC session profile. After completion of three CVAC sessions based on the third profile, the subject then returned to the first profile, with each profile be repeated in triad form. All CVAC sessions, regardless of the profile used, had a pressure ceiling corresponding to a specific tier. Subjects then progressed through tiers 2-5, and each tiered level included a maximum pressure ceiling that corresponded to an altitude of 4000 feet higher than the previous tier. A subject was not allowed to switch to the next higher tier until the subject had experienced fifteen CVAC sessions at the lower tier. Sham sessions (or control sessions) correspond to the cycling of the five tier levels but do not contain any meaningful pressure changes (e.g. pressure changes equivalent to altitude of 2000 feet with very few changes in duration), thus the subjects experience the CVAC session for the equivalent 20 minute session, but without the pressure changes and durations. In this study, profiles BRG (FIGS. 4, 8, 12, and 16), RBG (FIGS. 5, 9, 13, and 17), GRB (FIGS. 6, 10, 14, and 18) (tiers 2-5, respectively) were administered in sequential order for tiers 2-5 as described above. Sham sessions corresponding to tiers 2, 3, 4, and 5 (FIGS. 7, 11, 15, and 19) were administered where indicated and the graphical representations corresponding to pressures are not indicative of the pressure changes in the CVAC unit. The simulated graphical output was for control purposes to keep the subjects blinded to the sham sessions.

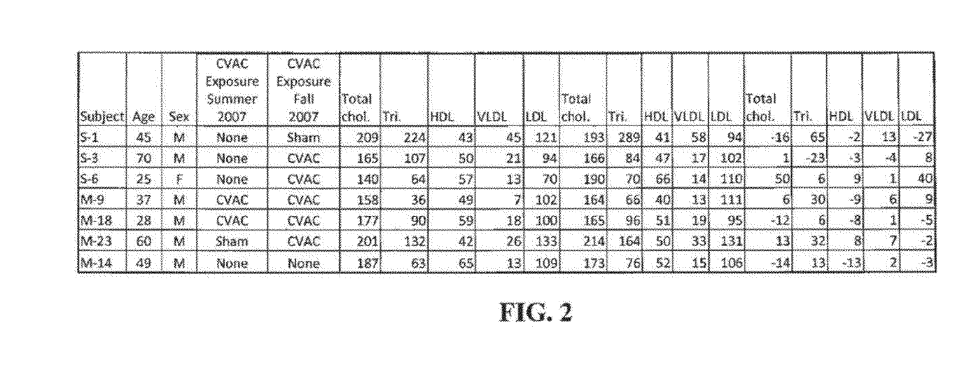

Blood samples were drawn prior to beginning the study period and after the final CVAC session at the end of the study period. Blood samples are analyzed for a variety of serum lipid levels including HDL, VLDL, and LDL. The results are summarized in FIG. 2.

### Example 6

During the foregoing studies listed in Examples 1 through 5, data was also recorded based on reports by participants of improved sensation and/or decreased pain as a result of one or more CVAC sessions. Specifically, such data was recorded for eight participants previously diagnosed with diabetic peripheral neuropathy. The participants reported a maximum pre-CVAC pain score within the range of 4-8 (of the eight, two participants did not report a maximum pre-CVAC pain score). Following one or more CVAC sessions, the participants reported a maximum post-CVAC pain score within a range of 0-3 (the two participants not reporting a maximum pre-CVAC pain score also did not report a maximum post-CVAC pain score). An increase or improvement in sensation was noted by the participants after one to five CVAC sessions. A decrease in pain was perceived by the participants after one to twenty-four CVAC sessions. For six of the eight participants for which data is available, four verbally reported an improvement in pain of 100%, suggesting a total alleviation of pain, and two participants verbally reported an improvement in pain of 50%, suggesting a significant alleviation of pain. Details of the participants and individualized data is set out in Table 3, below:

TABLE 3

| | | | | | No. of | No. of | Max | | |
|---|---|---|---|---|---|---|---|---|---|
| | | | | Max pre- | Sessions | Sessions | post- | | |
| | | | | CVAC | to | to | CVAC | % Pain | Max. |
| | Sex¹/ | Diagnosis³/ | Verbal Pain | Pain | Improved | Decreased | Pain | Improved | altitude |
| Participant | Race² | Year | Descriptor | Score | Sensation | Pain | Score | Verbally | (in feet) |
| 1 | M/C | DPN, 2002 | Pins and | 6 | 1 | 6 | 3 | 50% | 22,500 |
| | | | Needles | | | | | | |
| 2 | M/C | DPN/Foot | Burning | 6 | 3 | 5 | 3 | 50% | — |
| | | Ulcers, 2008 | | | | | | | |
| 3 | M/C | DPN, 1993 | Stabbing | 4 | 1 | 1 | 0 | 100% | 16,000 |
| 4 | M/C | DPN/Bilateral | Burning | 6 | 4 | 12 | 0 | 100% | 15,000 |
| | | BKA, 1993 | | | | | | | |
| 5 | F/C | DPN, 2005 | N/A | N/A | 3 | N/A | N/A | N/A | 20,000 |
| 6 | F/C | DPN, 2004 | Burning | 6 | 1 | 24 | 0 | 100% | — |
| 7 | M/C | DPN, 2007 | Stabbing & | 8 | 5 | 12 | 0 | 100% | — |
| | | | Burning to | | | | | | |
| | | | knees | | | | | | |
| 8 | M/B | DPN, 2000 | Severe | N/A | 4 | 4 | N/A | N/A | 18,500 |

¹“M” refers to male, “F” refers to female
²“C” refers to Caucasian, “B” refers to Black or African-American
³“DPN” refers to diabetic peripheral neuropathy, “BKA” refers to below-the-knee amputation

### Example 7

Data was collected in three men aged between 50 to 64 years of age, each of which was diagnosed with type 2 diabetes for two years or more and with diabetic peripheral neuropathy for six months or more. Each of the three participants received a series of CVAC sessions using one or more of the program profiles described herein.

Prior to receiving a CVAC session, a first male participant reported experiencing 100% bilateral loss of sensation from his knees to his toes, and pain in both feet. The first participant received three, forty minute CVAC sessions over a period of six weeks. Following the CVAC sessions, the first participant experienced restoration of sensory function in about four inches of his lower legs and 100% elimination of pain.

Prior to beginning a CVAC session, a second male participant, sixty-four years old, reported experiencing “pins and needles” type pain in the whole bottom of his foot, which he scored as 5-6 on a pain scale of 10. The second participant also reported decreased sensation along the bottom of his feet, a minor decrease in sensation on the top of his foot, and loss of sensation on all toes in both feet. The second participant was exposed to a first twenty-five minute CVAC session and then about sixty, twenty minute CVAC sessions. After the first CVAC session, the second participant reported noticing a return of some sensation in his lower limbs. After about sixty, twenty minute CVAC sessions, sensation returned primarily in the second participant's arch, heels, and toes, which sensation in the arch being restore to an estimated 100% of feeling. The second participant noted neuropathy remained in the ball of at least one foot, but that pain decreased to about 2-3 on the pain scale of 10.

Prior to beginning a CVAC session, a third male participant, sixty years old, reported 100% bilateral loss of sensation from his knees to his toes, but no pain in either limb. The third participant received a first forty-five minute CVAC session and about five, twenty-minute CVAC sessions. Following the first forty-five minute CVAC session, the third participant noticed a return of sensation, commenting that he could feel the socks on his feet. The return of sensation remained for about two to three days after receiving the CVAC session. After receiving the about five, twenty-minute CVAC sessions, the third participant noted complete return of bilateral sensation in his feet and legs. The returned sensation remains, and the individual continues a weekly CVAC program.

### Example 8

Ten participants diagnosed with adiposis dolorosa completed a study on the use of the CVAC methodology to improve pain associated with the adiposis dolorosa disorder. The participants included four men and six women, having an average age of 48±3.6 years and a range of 31 to 72 years old. All participants were non-Hispanic Caucasians, except for one male Hispanic. The average weight of participants was 88.7±8 kg, and the body mass index was 28.3±1.8 kg/m2. Half of the participants also carried a diagnosis of fibromyalgia.

The participants each received an initial CVAC session of twenty-five minutes on the day one and two twenty-minute CVAC sessions on days two through five. Each CVAC session included between about 300 and about 500 cyclic altitude changes in a twenty (or twenty-five) minute period, with an average rate of change of 30.5 meters per second (m/s). The approximate cumulative change within a twenty minute session was about 365,760 meters. The cyclic altitude changes were controlled by the automated CVAC system. The dynamic changes in altitude result in a pulsatile effect of pressure on the participants. On day one of the study, participants received the CVAC session in five stages, which collectively are Tier 1. Each of the five stages of Tier 1 was five minutes in duration. The maximum altitude for Tier 1 was 3,200 meters. One each of days two through 5, the participants received up to two twenty minute CVAC sessions on Tier 2, also with a maximum altitude of 3,200 meters. The average altitude for the CVAC sessions over the five day period was about 1, 828 meters. One participant only completed one CVAC session per day of the five day study and another participant only completed eight CVAC sessions over the five day study due to difficulty in equilibrating ear pressure.

On days one and five of the study, participants completed a questionnaire regarding pain severity, a scale for pain-related symptoms, a pain disability index, and a quality of life index. Each day of the study, participants completed a scale of pain severity. Following the CVAC sessions, the participant's current pain severity significantly decreased from 3.1±0.3 to 2.0±0.2 from a total of 5. The participants scores on the pain-related symptoms scale also significantly decreased on day five from day one from a score of 28.2±3.5 to a score of 25.2±2.9. The post-CVAC sessions also resulted in significantly reduced average, highest, and lowest pain levels on day five compared to day 1. Specifically, the average pain level was reduced from 5.6±0.6 to 4.2±0.6, the highest pain level was reduced from 7±0.7 to 5.7±0.7, and the lowest pain level was reduced from 4.4±0.5 to 3.4±0.4. Both the average level and lowest level of the daily scale for measuring pain severity showed both a significant linear decrease across the five days and quadratic patterns of change (flatter pain averages in the first days followed by larger decreases later) over the five day study. The highest level of the daily scale for measuring pain severity showed a significant linear decrease over the five days. As such, the five day study shows that the CVAC process, which includes cyclic pneumatic hypobaric compressions administered by a high-performance altitude simulator, results in decreased pain in people with adiposis dolorosa, and may also help in treating other chronic pain disorders.

### Example 9

To assess the efficacy of CVAC sessions, four individuals were administered CVAC sessions and their red blood cell counts hematocrit were subsequently measured and the levels recorded. Increases in red blood cell counts are indicative of CVAC session efficacy, and changes in hematocrit similarly indicate changes in erythropoiesis. For the study, CVAC sessions were administered to a group of four individuals for 40 minutes, 4 times a week, over an 8 week period. Red blood cell levels (RBC) were measured at 5 different intervals during the 8 week test period. The results of the study were as follows:

RBC mean increase: 4.7%

The increases in RBCs indicate that CVAC sessions were successful in positively modulating red blood cell counts as well as hematocrit, and both measurements are indicative of increased erythropoiesis. Thus, the administration of CVAC sessions successfully improved erythropoiesis in this 8 week study.

### Example 10

In the same study as Example 9, to assess the efficacy of CVAC sessions four individuals were administered CVAC sessions and their hematocrit was subsequently measured and the levels recorded. Changes in hematocrit indicate changes red blood cell concentration as well as indicating changes in erythropoiesis. For the study, CVAC sessions were administered to a group of four individuals for 40 minutes, 4 times a week, over an 8 week period. Hematocrit (HCT) was measured at 5 different intervals during the 8 week test period. The results of the study were as follows:

HCT mean increase: 5.3%

The increases in HCT, both alone in combination with the RBC increase as described in Example 9, indicate that CVAC sessions were successful in positively modulating hematocrit levels and are further indicative of increased erythropoiesis. Thus, the administration of CVAC sessions successfully improved erythropoiesis in this 8 week study.

In some embodiments, a method of increasing blood production prior to a traumatic brain injury (TBI) event includes administering at least one CVAC session, such as a CVAC session shown in at least one of FIG. 1A, 1B, 4-6, 8-10, 12-14, 16-18, or 20-43. The CVAC session has a start point, an end point and more than one target which is executed between the start point and the end point.

In some embodiments, the CVAC session includes a set of predetermined pressure targets with predetermined defined transitions. In some embodiments, the CVAC session can include any suitable number of cyclic altitude changes. For example, in some embodiments, the CVAC session can include 50, 100, 200, 300, 400, 500 or more cyclic altitude changes during a single CVAC session (e.g., during a CVAC session of 10, 15, 20, 25, 30, 35, 40, 45 or more minutes in length. In another example, the CVAC session can include between about 100 and about 500 cyclic altitude changes, between about 200 and about 400 cyclic altitude changes, or between about 300 and about 500 cyclic altitude changes.

In some embodiments, each CVAC session includes a plurality of targets executed within a short interval of the overall session duration. For example, in some embodiments, each CVAC session includes a plurality of targets executed during an interval of twenty (20) seconds, forty (40) seconds, one (1) minute, two (2) minutes, five (5) minutes, or any suitable interval, of the overall CVAC session duration. For example, during such an interval, the CVAC session can include about 10, 20, 30, 40, 50 or more cyclic pressure changes. In some embodiments, the method of increasing blood production prior to a traumatic brain injury (TBI) event includes administering a CVAC program and/or CVAC session as described in one of the Examples herein (e.g., Example 1).

In some embodiments, such a method further includes measuring efficacy of CVAC sessions via changes in physiological markers. In some embodiments, such measured physiological markers can include, for example, blood pressure, plasma lipid levels, HIF-1 a expression, VEGF production, Hematocrit, Erythropoietin (EPO) production, angiogenesis within tissues, blood-perfusion of tissues, and/or oxygenation of tissues.

In some embodiments, a method of increasing blood production prior to a traumatic brain injury (TBI) event includes administering at least one CVAC session, such as a CVAC session shown in at least one of FIG. 1A, 1B, 4-6, 8-10, 12-14, 16-18, or 20-31. The CVAC session has a start point, an end point and more than one target which is executed between the start point and the end point. In some embodiments, the CVAC session includes a set of predetermined pressure targets with predetermined defined transitions. In some embodiments, the CVAC session can include any suitable number of cyclic altitude changes. For example, in some embodiments, the CVAC session can include 50, 100, 200, 300, 400, 500 or more cyclic altitude changes during a single CVAC session (e.g., during a CVAC session of 10, 15, 20, 25, 30, 35, 40, 45 or more minutes in length. In another example, the CVAC session can include between about 100 and about 500 cyclic altitude changes, between about 200 and about 400 cyclic altitude changes, or between about 300 and about 500 cyclic altitude changes. In some embodiments, each CVAC session includes a plurality of targets executed within a short interval of the overall session duration. For example, in some embodiments, each CVAC session includes a plurality of targets executed during an interval of twenty (20) seconds, forty (40) seconds, one (1) minute, two (2) minutes, five (5) minutes, or any suitable interval, of the overall CVAC session duration. For example, during such an interval, the CVAC session can include about 10, 20, 30, 40, 50 or more cyclic pressure changes. In some embodiments, the method of increasing blood production prior to a traumatic brain injury (TBI) event includes administering a CVAC program and/or CVAC session as described in one of the Examples herein (e.g., Example 1). In some such methods, the CVAC session is administered at defined intervals prior to the TBI event. In other such methods, the CVAC session is administered at random intervals prior to the TBI event. In some embodiments, the administering at least one CVAC session includes administering at least one pharmaceutical compound. In some embodiments, a user can modulate one or more parameters of a CVAC session.

In some embodiments, a method of stem cell treatment in a patient subsequent to a traumatic brain injury (TBI) event includes administering at least one CVAC session, such as a CVAC session shown in at least one of FIG. 1A, 1B, 4-6, 8-10, 12-14, 16-18, or 20-43. The CVAC session has a start point, an end point and more than one target which is executed between the start point and the end point. In some embodiments, the CVAC session includes a set of predetermined pressure targets with predetermined defined transitions. In some embodiments, the CVAC session can include any suitable number of cyclic altitude changes. For example, in some embodiments, the CVAC session can include 50, 100, 200, 300, 400, 500 or more cyclic altitude changes during a single CVAC session (e.g., during a CVAC session of 10, 15, 20, 25, 30, 35, 40, 45 or more minutes in length. In another example, the CVAC session can include between about 100 and about 500 cyclic altitude changes, between about 200 and about 400 cyclic altitude changes, or between about 300 and about 500 cyclic altitude changes.

In some embodiments, each CVAC session includes a plurality of targets executed within a short interval of the overall session duration. For example, in some embodiments, each CVAC session includes a plurality of targets executed during an interval of twenty (20) seconds, forty (40) seconds, one (1) minute, two (2) minutes, five (5) minutes, or any suitable interval, of the overall CVAC session duration. For example, during such an interval, the CVAC session can include about 10, 20, 30, 40, 50 or more cyclic pressure changes. In some embodiments, the method of increasing blood production prior to a traumatic brain injury (TBI) event includes administering a CVAC program and/or CVAC session as described in one of the Examples herein (e.g., Example 1). In some embodiments, such a method further includes administering at least one growth factor. In some embodiments, the at least one growth factor is G-CSF, EPO, or a combination of G-CSF and EPO. In some embodiments, the method includes measuring efficacy of the at least one CVAC session via changes in physiological markers. In some embodiments, the physiological marker measured can be a Mean Fluorescence Index (MFI), Mean Reticulocyte Volume (MRV), VEGF production, Hematocrit, Erythropoietin (EPO) production, and/or oxygenation of tissues in the patient. In some embodiments, the method includes administering at least one CVAC session prior to a TBI event.

In some embodiments, a method of treating inflammation or swelling or a combination thereof in a patient subsequent to a traumatic brain injury (TBI) event includes administering at least one CVAC session, the CVAC session having a start point, an end point and more than one target which is executed between said start point and said end point. In some embodiments, the CVAC session includes a set of predetermined pressure targets with predetermined defined transitions. In some embodiments, the CVAC session can include any suitable number of cyclic altitude changes. For example, in some embodiments, the CVAC session can include 50, 100, 200, 300, 400, 500 or more cyclic altitude changes during a single CVAC session (e.g., during a CVAC session of 10, 15, 20, 25, 30, 35, 40, 45 or more minutes in length. In another example, the CVAC session can include between about 100 and about 500 cyclic altitude changes, between about 200 and about 400 cyclic altitude changes, or between about 300 and about 500 cyclic altitude changes.

In some embodiments, each CVAC session includes a plurality of targets executed within a short interval of the overall session duration. For example, in some embodiments, each CVAC session includes a plurality of targets executed during an interval of twenty (20) seconds, forty (40) seconds, one (1) minute, two (2) minutes, five (5) minutes, or any suitable interval, of the overall CVAC session duration. For example, during such an interval, the CVAC session can include about 10, 20, 30, 40, 50 or more cyclic pressure changes. In some embodiments, the method of increasing blood production prior to a traumatic brain injury (TBI) event includes administering a CVAC program and/or CVAC session as described in one of the Examples herein (e.g., Example 1). In some embodiments, the method further includes measuring an efficacy of CVAC sessions via changes in physiological markers. In some embodiments, the physiological marker measured can be hematocrit, erythropoietin (EPO) production, blood gas composition, oxygenation of tissues, angiogenesis within tissues, blood-perfusion of tissues, and/or extent of tissue necropsy following the onset of inflammation or swelling. In some embodiments, the CVAC session is administered at defined intervals following the TBI event. In some embodiments, the CVAC session is administered at random intervals following the TBI event. In some embodiments, the method further includes administering at least one pharmaceutical compound. In some embodiments, the method further includes administering at least one non-pharmaceutical therapy.

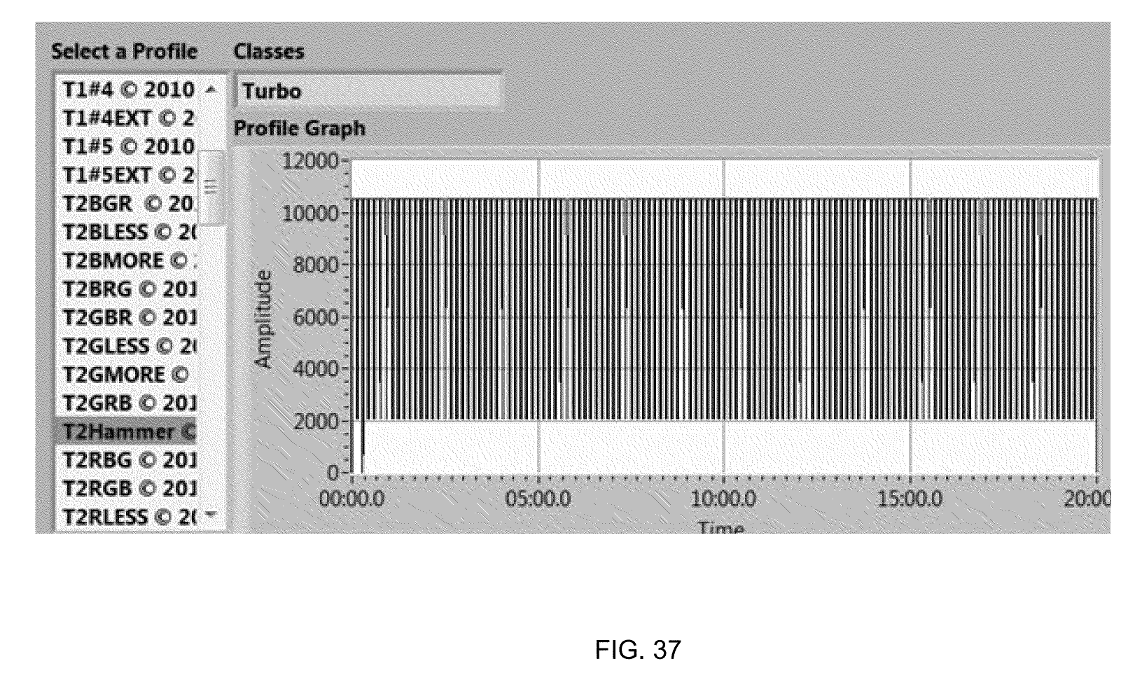

The methods shown and described herein include administering a cyclic variations in altitude conditioning (CVAC) session to expose a user disposed within a pressure vessel to varying cyclic patterns of transitions between simulated altitudes. For example, as shown in FIGS. 1A and 1B, the CVAC session profile includes a highly variable pre-determined arrangement of pressure targets therein. In some embodiments, a CVAC session can be administered to execute pressure targets in a pattern having a higher degree of repeating pressure sequences, such as is shown in FIGS. 37-41. For example, FIG. 37 depicts a graph of a CVAC session profile of pressure targets with a start point at ambient pressure, and end point at ambient pressure, and a plurality of pressure targets between 2,000 ft and 10,500 ft. As shown in FIG. 37, the profile pressure target pattern includes, for a first time period achieving and maintaining a maximum pressure target for a non-zero duration (e.g., about 10 seconds), and then quickly transitioning to a lower pressure target before immediately returning to the maximum pressure target. This pattern is repeated, with variations in the lower pressure target for each transition from the maximum pressure target, as shown in FIG. 37. As shown in FIG. 37, in some embodiments, at least one pressure target between the start point and the end point can be at zero feet above ambient.

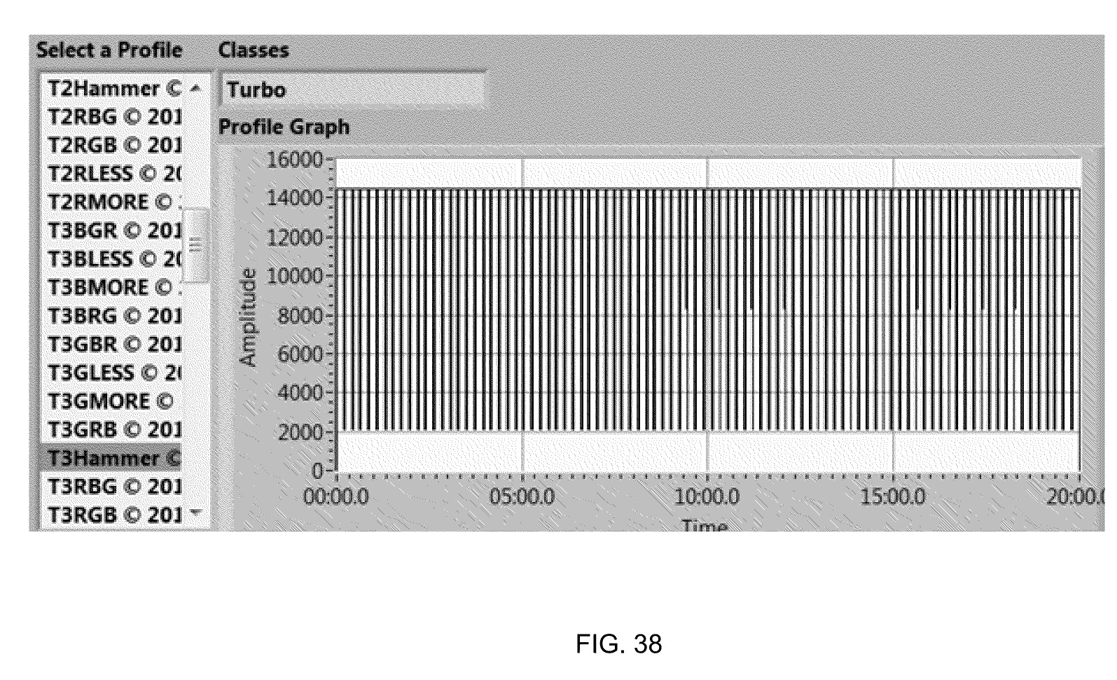

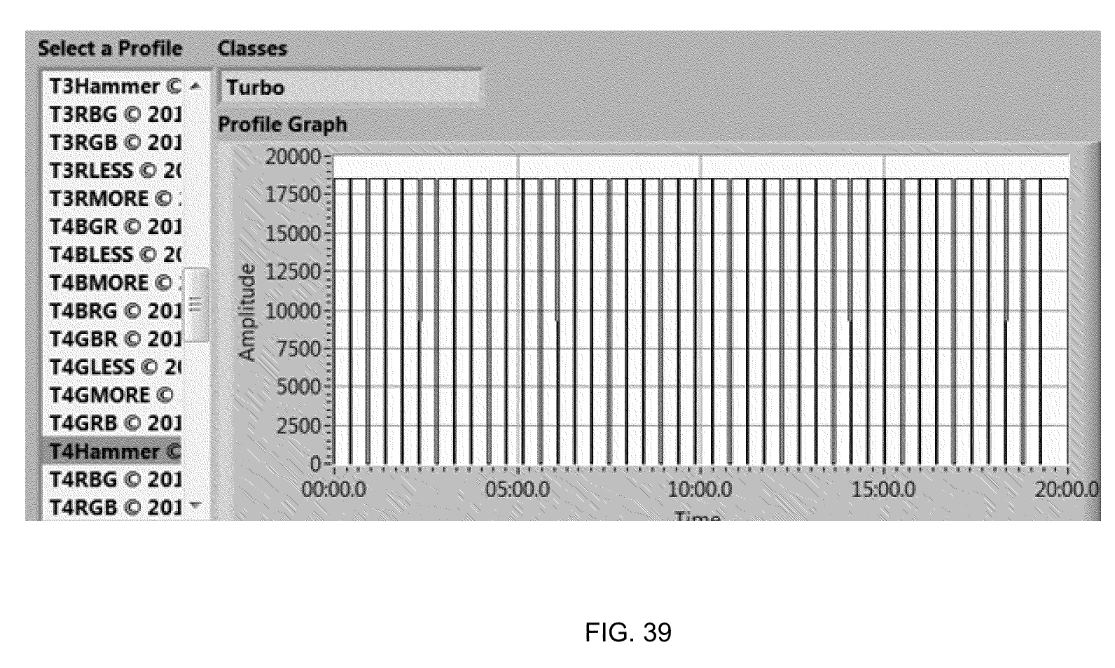

FIGS. 38, 39 and 41 are examples of CVAC profiles with patterns similar in some respects to that shown in FIG. 37. For example, in FIG. 38, the CVAC profile pressure targets with a start point at ambient pressure, and end point at ambient pressure, and a plurality of pressure targets between 2,000 ft and 14,500 ft in which the maximum pressure target of 14,500 ft is repeatedly achieved and maintained for a non-zero duration (e.g., about 10 to 20 seconds), before transitioning to a lower pressure target and immediately transitioning back to the maximum pressure target. This pattern is repeated, with some variations in the lower pressure targets, throughout the CVAC session, which can be 20 minutes in length. FIG. 39 depicts another CVAC profile with a pattern similar in many respects to that shown in FIG. 38, except that the minimum pressure target is zero (0) ft and the maximum pressure target is 18,500 ft, and the duration at which the maximum pressure target is maintained is increased (e.g., lasting from about 20 seconds to about 30 seconds). As also shown in FIG. 38, after a transition from the maximum pressure target to a lower pressure target has occurred, the lower transition target can be maintained for a short duration (e.g., about 10 to 15 seconds), as shown on the graph between time periods 00:40.0 and 01:00.0.

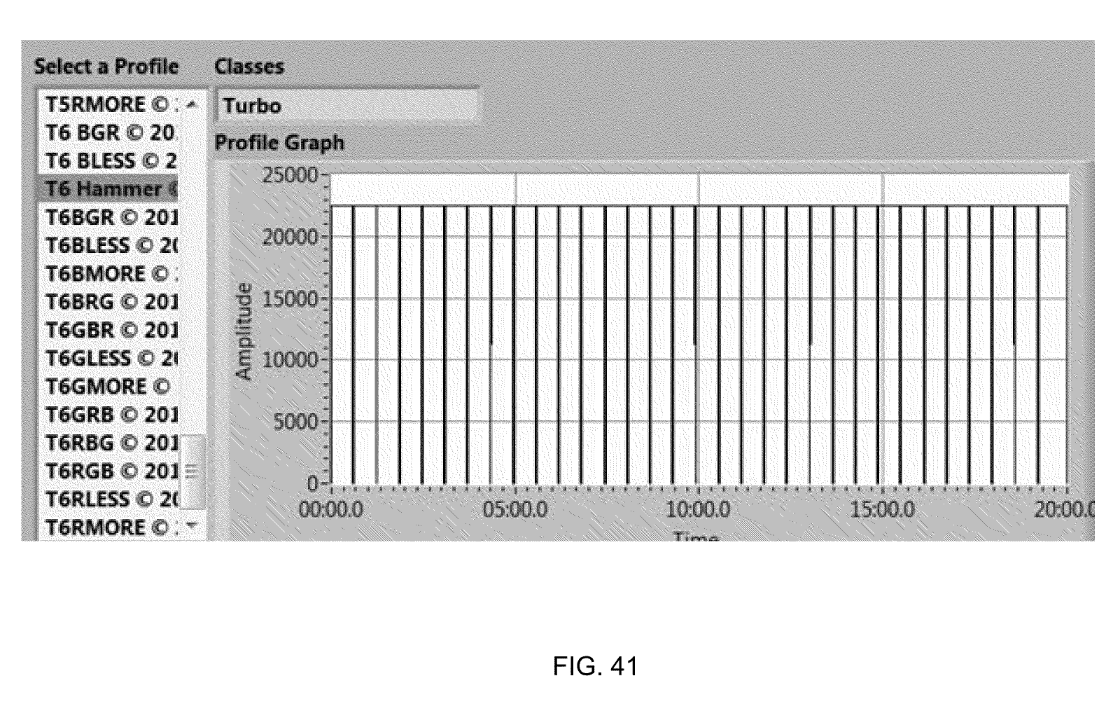

FIG. 41 depicts a CVAC session according to an embodiment that can include a pattern similar in many respects to that shown in FIGS. 38 and 39, except that the maximum pressure target is 22,500 ft and the minimum pressure target is zero (0) ft.

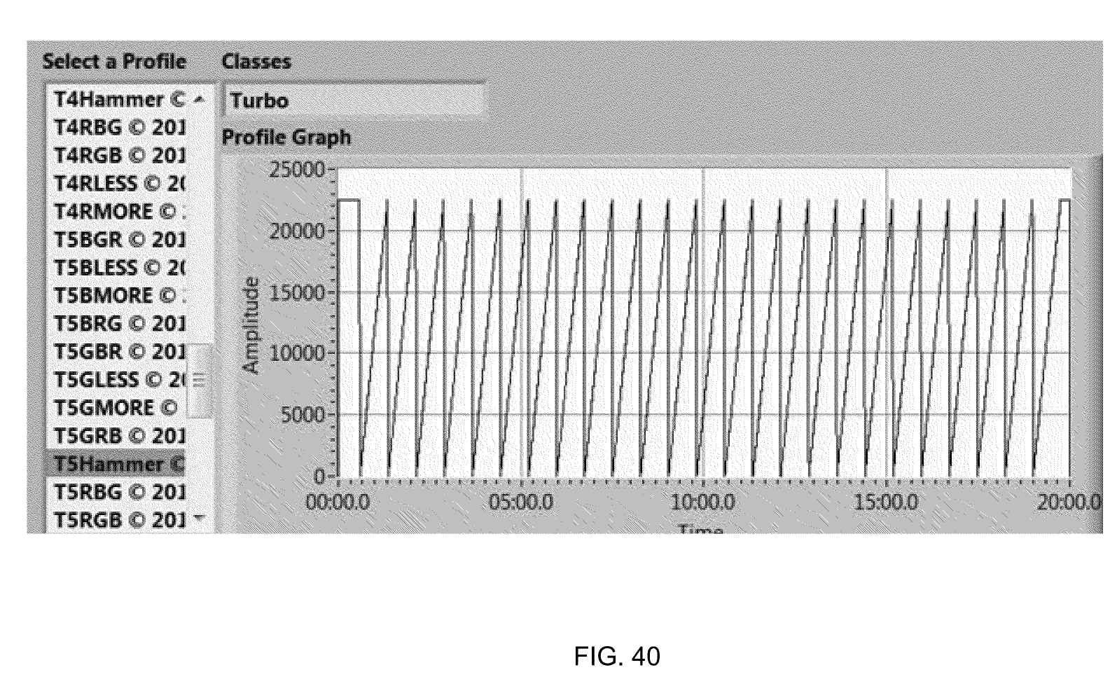

Returning to FIG. 40, in some embodiments, a CVAC session can include a maximum pressure target of 22, 500 ft, a minimum pressure target of about zero (0) ft, and a repeating pattern of transitions therebetween in which the transition from the minimum pressure target to the maximum pressure target is achieved over a period of about 40 seconds, followed by an almost immediate (e.g., about 1, about 5, or about 10 seconds) transition from the maximum pressure target to the minimum pressure target.

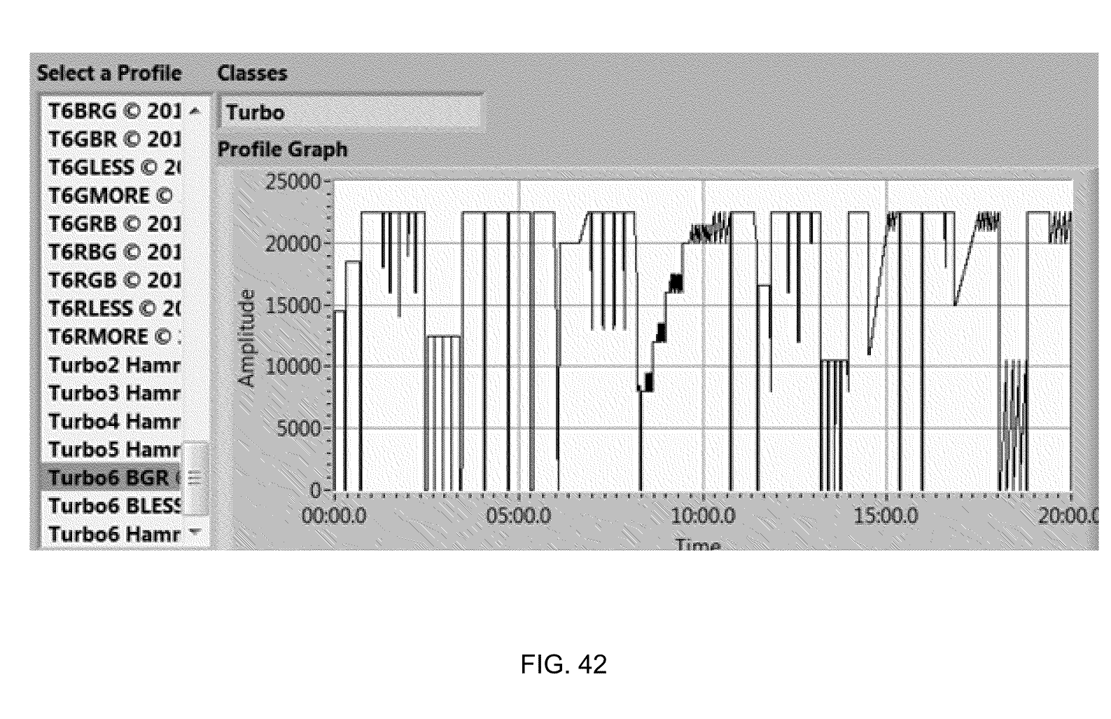

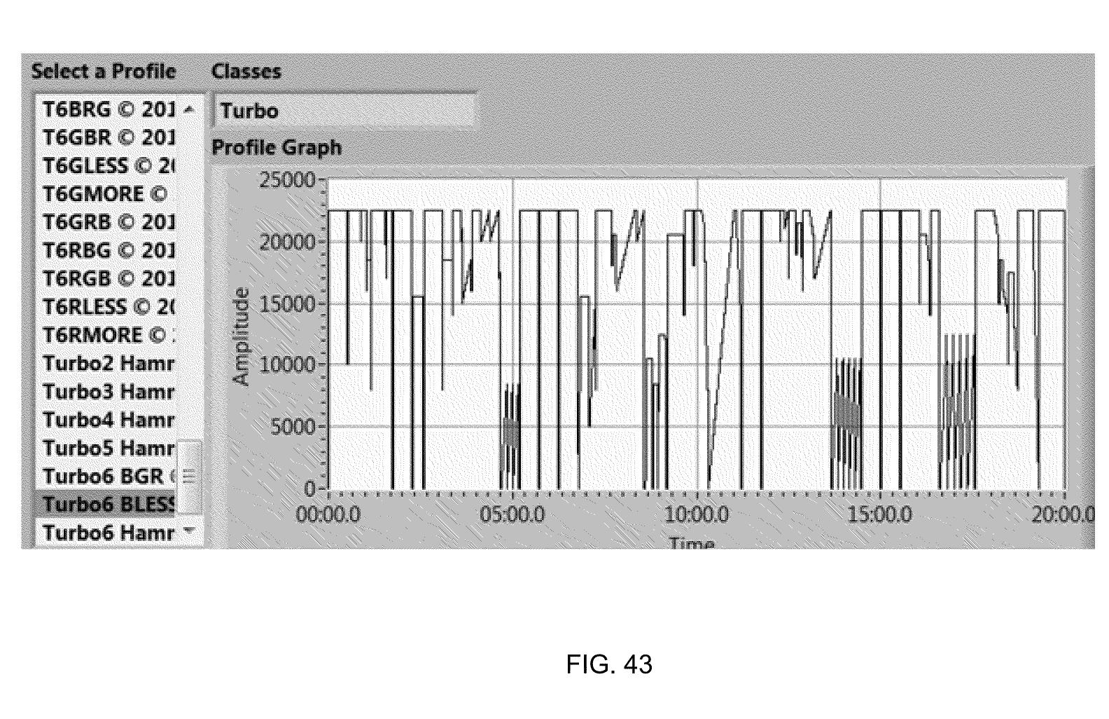

Additionally, although CVAC sessions have been shown and described herein as including a variable pattern of pressure targets between a non-zero minimum pressure target (e.g., about 2,000 ft or about 2,500 ft) and a maximum pressure target, in some embodiments, a CVAC session includes a variable pattern of pressure targets, between a start point and end point each at ambient pressure, that includes a minimum pressure target of ambient pressure (e.g., zero feet), as shown in FIGS. 42 and 43. Such CVAC sessions can also include intervals in which the maximum pressure target, or a pressure target between the maximum and the minimum, is maintained for about 10 seconds, about 20 seconds, about 30 seconds, or about 40 seconds, as also shown in FIGS. 42 and 43.

Example embodiments of the methods and systems of the present invention have been described herein. As noted elsewhere, these example embodiments have been described for illustrative purposes only, and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments, but should be defined only in accordance with the following claims and their equivalents.

All references cited within this document are incorporated by reference in their entirety.

What is claimed is:

1. A method of increasing blood production prior to a traumatic brain injury (TBI) event comprising:
administering at least one CVAC session, the CVAC session having a start point, an end point and more than one target which is executed between the start point and the end point.

2. The method of claim 1, further comprising measuring efficacy of CVAC sessions via changes in physiological markers.

3. The method of claim 2, wherein the physiological marker measured is selected from the group consisting of: blood pressure; plasma lipid levels; HIF-1 a expression;
VEGF production; Hematocrit; Erythropoietin (EPO) production; angiogenesis within tissues; blood-perfusion of tissues; oxygenation of tissues; and combinations thereof.

4. The method of claim 1, wherein the CVAC session is administered at defined intervals prior to the TBI event.

5. The method of claim 1, wherein said CVAC session is administered at random intervals prior to the TBI event.

6. The method of claim 1, further comprising administering at least one pharmaceutical compound.

7. The method of claim 1, wherein a user can modulate one or more parameters of a session.

8. A method of stem cell treatment in a patient subsequent to a traumatic brain injury (TBI) event comprising:
administering at least one CVAC session, the CVAC session having a start point, an end point and more than one target which is executed between the start point and the end point.

9. The method of claim 8, further comprising administering at least one growth factor.

10. The method of claim 9, wherein the at least one growth factor is G-CSF, EPO, or a combination of G-CSF and EPO

11. The method of claim 8, further comprising measuring efficacy of the at least one CVAC session via changes in physiological markers.

12. The method of claim 11, wherein the physiological marker measured is selected from the group consisting of: Mean Fluorescence Index (MFI); Mean Reticulocyte Volume (MRV); VEGF production; Hematocrit; Erythropoietin (EPO) production; oxygenation of tissues in the patient; and combinations thereof.

13. The method of claim 8, further comprising administering at least one CVAC session prior to a TBI event.

14. A method of treating inflammation or swelling or a combination thereof in a patient subsequent to a traumatic brain injury (TBI) event comprising:
administering at least one CVAC session, the CVAC session having a start point, an end point and more than one target which is executed between said start point and said end point.

15. The method of claim 14, further comprising measuring efficacy of CVAC sessions via changes in physiological markers.

16. The method of claim 15, wherein the physiological marker measured is selected from the group consisting of: hematocrit; erythropoietin (EPO) production; blood gas composition; oxygenation of tissues; angiogenesis within tissues; blood-perfusion of tissues; or extent of tissue necropsy following the onset of inflammation or swelling; and combinations thereof.

17. The method of claim 14, wherein said CVAC session is administered at defined intervals following the TBI event.

18. The method of claim 14, wherein said CVAC session is administered at random intervals following the TBI event.

19. The method of claim 14, further comprising administering at least one pharmaceutical compound.

20. The method of claim 14, further comprising administering at least one non-pharmaceutical therapy.
